SEQUENCE LISTING (103 sequences)

SEQ ID NO: 1 - DNA, 948 bp, synthetic construct
  source 1..948: mol_type = other DNA
gccaccatgacaagctgccacatcgccgaggagcacatccagaaagtcgccatttttgggggaactcacggtaacgaactcacaggggtcttcctggtgaagcactggctcgagaacggcgcagaaatccagagaaccggactggaggtgaaacccttcattacaaatcctcgggccgtcaagaaatgcactcgctacatcgactgtgatctgaaccggatttttgatctggaaaatctcggcaagaaaatgtccgaggacctgccatacgaagtgaggagagctcaggagatcaaccacctcttcggacccaaggacagcgaagattcctatgacatcatttttgatctgcataacaccacatcaaatatggggtgcaccctgatcctcgaggacagccgcaacaatttcctgatccagatgtttcactatattaagacaagtctggcaccactcccctgttacgtgtatctgattgagcatccctctctcaagtacgctactacccgaagtatcgcaaaatatcctgtggggattgaagtcggtcctcagccacagggagtcctgcgagccgatatcctcgaccagatgaggaagatgatcaaacatgctctggatttcattcaccacttcaacgagggcaaggagttccccccttgcgccatcgaggtgtacaagatcattgaaaaagtcgattatcctcgggacgagaacggcgaaattgccgctatcattcacccaaatctgcaggaccaggattggaagcccctccatcctggggatccaatgttcctgacactcgacggtaaaactatcccactgggcggagactgtaccgtgtaccccgtgtttgtcaatgaggcagcctactatgagaagaaagaagctttcgccaaaacaacaaaactcactctcaatgctaaatctattcggtgctgcctccactga

SEQ ID NO: 2 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 3 - DNA, 399 bp, synthetic construct
  source 1..399: mol_type = other DNA
ccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcc

SEQ ID NO: 4 - DNA, 260 bp, synthetic construct
  source 1..260: mol_type = other DNA
ccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 5 - DNA, 282 bp, synthetic construct
  source 1..282: mol_type = other DNA
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 6 - protein, 313 aa, Homo sapiens
MTSCHIAEEHIQKVAIFGGTHGNELTGVFLVKHWLENGAEIQRTGLEVKPFITNPRAVKKCTRYIDCDLNRIFDLENLGKKMSEDLPYEVRRAQEINHLFGPKDSEDSYDIIFDLHNTTSNMGCTLILEDSRNNFLIQMFHYIKTSLAPLPCYVYLIEHPSLKYATTRSIAKYPVGIEVGPQPQGVLRADILDQMRKMIKHALDFIHHFNEGKEFPPCAIEVYKIIEKVDYPRDENGEIAAIIHPNLQDQDWKPLHPGDPMFLTLDGKTIPLGGDCTVYPVFVNEAAYYEKKEAFAKTTKLTLNAKSIRCCLH

SEQ ID NO: 7-44 - skipped (38 entries)

SEQ ID NO: 45 - RNA, 13 nt, synthetic construct
  source 1..13: mol_type = other RNA
gccgccaccatgg

SEQ ID NO: 46-50 - skipped (5 entries)

SEQ ID NO: 51 - DNA, 592 bp, synthetic construct
  source 1..592: mol_type = other DNA
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

SEQ ID NO: 52-100 - skipped (49 entries)

SEQ ID NO: 101 - DNA, 56 bp, synthetic construct
  source 1..56: mol_type = other DNA
aataaaggaaatttattttcattgcaatagtgtgttggaattttttgtgtctctca

SEQ ID NO: 102 - DNA, 130 bp, synthetic construct
  source 1..130: mol_type = other DNA
ctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 103 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2026/0048146 A1
(43) Pub. Date: Feb. 19, 2026

(54) METHODS AND COMPOSITIONS FOR TREATING LEUKODYSTROPHIES

(71) Applicant: BRIDGEBIO GENE THERAPY LLC, San Francisco, CA (US)

(72) Inventors: Genevieve LAFORET, Bolton, MA (US); Adam SHAYWITZ, Irvine, CA (US); Kathleen M. KIRBY, Andover, MA (US)

(21) Appl. No.: 18/700,508

(22) PCT Filed: Oct. 12, 2022
(86) PCT No.: PCT/US2022/077971

§ 371 Date: Apr. 11, 2024

Related U.S. Application Data
(60) Provisional application No. 63/254,885, filed on Oct. 12, 2021.
(60) Provisional application No. 63/352,049, filed on Jun. 14, 2022.

(51) Int. Cl. A61K 48/00; A61K 38/50; C12N 15/86

(52) U.S. Cl. CPC A61K 48/0058; A61K 38/50; A61K 48/0066; A61K 48/0075; C12N 15/86; C12Y 305/01015; C12N 2750/14143; C12N 2800/22; C12N 2830/008

(57) ABSTRACT

Disclosed herein are recombinant adeno-associated viral vectors expressing aspartoacylase (ASPA) protein and related uses for treating leukodystrophies.

### CROSS REFERENCE TO RELATED APPLICATIONS

The present Application is a U.S. National Stage Application filed under 35 U.S.C. § 371 of International Application No. PCT/US2022/077971, filed Oct. 12, 2022, which claims the benefit of priority to U.S. Provisional Application No. 63/254,885, filed on Oct. 12, 2021, and U.S. Provisional Application No. 63/352,049, filed Jun. 14, 2022, the contents of each of which is hereby incorporated by reference in its entirety for all purposes.

### REFERENCE TO AN ELECTRONIC SEQUENCE LISTING

The contents of the electronic sequence listing (ASPA_004_02US_SeqList_ST26.xml; Size: 32,465 bytes; and Date of Creation: Apr. 11, 2024) is herein incorporated by reference in its entirety

### BACKGROUND

Leukodystrophies are a group of rare, primarily inherited neurological disorders that result from the abnormal production, processing, or development of myelin and other components of central nervous system (CNS) white matter, such as cells called oligodendrocytes and astrocytes. Reduced myelin function results in progressive loss of neurological function.

More than 50 different leukodystrophies have been identified, among them: Alexander disease autosomal dominant leukodystrophy with autonomic diseases (ADLD), Canavan disease, cerebrotendinous xanthomatosis (CTX), metachromatic leukodystrophy (MLD), Pelizaeus-Merzbacher disease, and Refsum disease. The specific symptoms of leukodystrophy vary widely across the different disease types.

There are no treatments for leukodystrophies that significantly alter the course of the disease. Instead, treatments are targeted at easing symptoms and preserving some neurological function. Thus, there is an unmet need for developing new therapeutics for leukodystrophy disease types, such as, Canavan disease.

### SUMMARY

The disclosure provides methods of treating subjects with leukodystrophy with a gene therapy. The gene therapy may comprise administration of a recombinant adeno-associated viral vector comprising a nucleic acid molecule encoding aspartoacylase (ASPA).

In an aspect, the present disclosure provides a method comprising: administering to a subject a therapeutically effective amount of a recombinant adeno-associated virus (rAAV) vector, wherein the rAAV vector comprises: (i) a nucleic acid molecule comprising at least one AAV inverted terminal repeat (ITR) and (ii) a non-AAV nucleotide sequence encoding aspartoacylase (ASPA), wherein the non-AAV nucleotide sequence is operably linked to a promoter.

In a related aspect, the present disclosure provides a method of expressing aspartoacylase (ASPA) in a subject in need thereof, comprising: administering to the subject a therapeutically effective amount of a recombinant adeno-associated virus (rAAV) vector, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In another aspect, the present disclosure provides a method of increasing the level of aspartoacylase (ASPA) in a subject in need thereof, comprising: administering to the subject a therapeutically effective amount of a recombinant adeno-associated virus (rAAV) vector, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In a further aspect, the present disclosure provides a method of treating leukodystrophy in a subject in need thereof, comprising: administering to the subject a therapeutically effective amount of a recombinant adeno-associated virus (rAAV) vector, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In another aspect, the present disclosure provides a method of treating Canavan disease in a subject in need thereof, comprising: administering to the subject a therapeutically effective amount of a recombinant adeno-associated virus (rAAV) vector, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In another aspect, the present disclosure provides a use of a therapeutically effective amount of a recombinant adeno-associated virus (rAAV) vector in the treatment of a leukodystrophy, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In another aspect, the present disclosure provides a use of a therapeutically effective amount of a recombinant adeno-associated virus (rAAV) vector in the treatment of Canavan disease, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In a further aspect, the present disclosure provides a composition for treating a leukodystrophy, comprising a therapeutically effective amount of a recombinant adeno-associated virus (rAAV) vector in the treatment of Canavan disease, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In a further aspect, the present disclosure provides a composition for treating Canavan disease, comprising a therapeutically effective amount of a recombinant adeno-associated virus (rAAV) vector in the treatment of Canavan disease, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In another aspect, the present disclosure provides a method of using a therapeutically effective amount of a recombinant adeno-associated virus (rAAV) vector in the manufacture of a medicament for or production of a substance for treating a leukodystrophy, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In another aspect, the present disclosure provides a method of using a therapeutically effective amount of a recombinant adeno-associated virus (rAAV) vector in the manufacture of a medicament for or production of a substance for treating Canavan disease, wherein the rAAV vector comprises: (i) a nucleic acid molecule comprising at least one AAV inverted terminal repeat (ITR) and (ii) a non-AAV nucleotide sequence encoding ASPA, wherein the non-AAV nucleotide sequence is operably linked to a promoter.

In some embodiments of any of the preceding aspects, the leukodystrophy is associated with a condition selected from the group consisting of Canavan disease, adrenomyeloneuropathy, Alexander disease, cerebrotendinous xanthomatosis, Krabbe disease, metachromatic leukodystrophy, adrenoleukodystrophy, Pelizaeus-Merzbacher disease, and Refsum disease. In some embodiments, the leukodystrophy is associated with Canavan disease.

In some embodiments of any of the preceding aspects, the therapeutically effective amount is in the range of about 10¹³ vector genomes per kilogram (vg/kg) to about 10¹⁵ vg/kg.

In some embodiments, the therapeutically effective amount is in the range of about 10¹⁴ vg/kg to about 5×10¹⁴ vg/kg. In some embodiments, the therapeutically effective amount is at least about 1.32×10¹⁴ vg/kg. In some embodiments, the therapeutically effective amount is about 3×10¹⁴ vg/kg.

In some embodiments of any of the preceding aspects, the rAAV is administered via intravenous infusion.

In some embodiments of any of the preceding aspects, the subject is less than, or equal to, 30 months of age.

### BRIEF DESCRIPTION OF THE DRAWINGS

FIG. 1 shows the design of the self-complementary AAV (scAAV) vector used in the study of Example 1, referred to as “scAAV9-CB6-hASPAopt”, comprising a CB6 promoter and a codon optimized transgene encoding human ASPA protein. Abbreviations: CMV: cytomegalovirus; IE: immediate-early; ITR: inverted terminal repeat.

FIG. 2 shows the weights of animals treated with mid- and high-dose regimens of BBP-812. Weights were taken every other day for the first 32 days and biweekly thereafter (n=10 each).

FIG. 3 shows magnetic resonance spectrometry analysis of brain showing total N-acetylaspartic acid (tNAA) levels normalized against total creatine (tCr) (n=3).

FIG. 4 shows motor behavior testing time points at post-natal day (PND) 27, 90, 180, and 364 in wild type (WT) mice, untreated mice, and mice treated with BBP-812.

FIG. 5A and FIG. 5B show brain histology in wild type and Aspa−/− (knockout, KO) mice treated at PND 1 with BBP-812 at 2.6×10¹³, 8.8×10¹³, and 2.6×10¹⁴ vector genomes per kilogram (vg/kg). (CA3: cornu ammonis 3; Ce: cerebellum; Cx: cortex; DG: dentate gyrus).

FIG. 6 shows eleven different regions of the CNS that were analyzed for vector genome copy number per cell using ddPCR at Day 364 post vector administration. (BS: brain stem; CBL: cerebellum; CSC: cervical spinal cord; CNS: central nervous system; Cx: cortex; ddPCR: droplet digital polymerase chain reaction; HPC: hippocampus; LMN: lamina tecti; LSC: lumbar spinal cord; MB: midbrain).

FIG. 7A and FIG. 7B show brain histology in wildtype Aspa−/− mice treated at PND1 (post-natal day 1) with BBP-812.

FIG. 8 shows the locations of samples taken and used to determine biodistribution of BBP-812 and expression of the hASPAOpt transgene.

FIGS. 9A-9E show assessment of BBP-812 in the brain (FIG. 9A), spinal cord (FIG. 9B), liver (FIG. 9C), heart (FIG. 9D), and kidney (FIG. 9E). For brain and spinal cord, each individual tissue region is plotted as an individual data point.

FIG. 10A and FIG. 10B show measurements of creatine kinase (FIG. 10A) and lactate dehydrogenase (FIG. 10B).

FIG. 11 shows results from an ELISA that shows reactivity of splenocytes from vehicle (V), 1×10¹⁴ vg/kg treated animals (L), or 3×10¹⁴ vg/kg treated mice (H) to media (negative control), ConA (positive control), and peptide pools to AAV9 and ASPA. (SFU: spot forming units).

FIG. 12 shows vector genomes (top panel) and transgene RNA (bottom panel) detected in the 3×10¹⁴ vg/kg dose group from 4-, 12-, and 24-week biodistribution samples.

FIG. 13 shows the timeline and overall design of Phase 1/2 study of gene therapy to treat Canavan disease. (d: days; DSMC: Data and Safety Monitoring Committee; kg: kilogram; mo: month; N: number of participants; vg: vector genomes).

FIG. 14 shows the study dose finding, observation, and enrollment expansion sequence.

FIG. 15 shows the glucocorticoid prophylaxis and tapering regimen.

### DETAILED DESCRIPTION

The present disclosure relates to recombinant adeno-associated virus (AAV) vectors that are engineered to express aspartoacylase (ASPA) in a subject in need thereof. Aspects of the disclosure relate to methods for treating neurodegenerative disease (e.g., leukodystrophies, such as Canavan disease) in a subject in need thereof. Methods provided herein, in some embodiments, involve modulating N-acetylaspartate (NAA) levels in the subject. NAA is metabolized by aspartoacylase (ASPA) into acetate and L-aspartate.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

### Recombinant AAV Vectors and Particles

In an aspect, the present disclosure provides a viral vector for delivery of a nucleic acid sequence encoding ASPA to a cell, such as to a cell in need of treatment. Thus, in some embodiments, the present disclosure relates to a recombinant adeno-associated virus (rAAV) vector comprising a nucleic acid molecule comprising at least one AAV inverted terminal repeat (ITR) and a non-AAV nucleotide sequence (also referred to as a heterologous polynucleotide) encoding ASPA, with the non-AAV nucleotide sequence operably linked to a promoter.

As used herein, the term “operable linkage” or “operably linked” refers to a physical or functional juxtaposition of the components so described as to permit them to function in their intended manner. In the example of an expression control element (such as a promoter or enhancer) in operable linkage with a polynucleotide, the relationship is such that the control element modulates expression of the nucleic acid. More specifically, for example, two deoxyribonucleic acid (DNA) sequences operably linked means that the two DNAs are arranged (cis or trans) in such a relationship that at least one of the DNA sequences is able to exert a physiological effect upon the other sequence. “Operably linked” may mean that the nucleic acid sequences being linked are contiguous, or substantially contiguous, and, where necessary to join two protein coding regions, contiguous and in reading frame.

In some embodiments, an rAAV vector expresses an ASPA protein that is a human ASPA protein. In some cases, the ASPA protein expressed by an rAAV vector described herein is a native (e.g., wild-type) ASPA protein. The ASPA protein or polypeptide encoded by the nucleotide sequence includes full-length native sequences, as with a naturally occurring ASPA protein, as well as functional subsequences, modified forms, or sequence variants so long as the subsequence, modified form, or variant retains some degree of functionality of the native full-length ASPA protein. In methods and uses of the present disclosure, ASPA proteins and polypeptides encoded by the nucleotide sequences in an rAAV vector can be, but are not required to be, identical to the endogenous ASPA protein that is defective, or whose expression is insufficient, or deficient in the treated subject. In some embodiments, ASPA comprises an amino acid sequence of SEQ ID NO: 6, or an amino acid sequence with at least about 90%, at least about 95%, at least about 96%, at least about 97%, at least about 98%, at least about 99%, at least about 99.5%, or 100% identity to SEQ ID NO: 6.

In some embodiments, the non-AAV nucleotide sequence (e.g., heterologous sequence) encoding an ASPA protein is the wild-type ASPA gene sequence. In some embodiments, the non-AAV nucleotide sequence (e.g., heterologous sequence) encoding an ASPA protein has been codon-optimized with respect to the wild-type ASPA gene sequence. In some embodiments, an ASPA-encoding nucleotide sequence of the present disclosure is a codon-optimized sequence and comprises or consists of SEQ ID NO: 1. In other embodiments, the non-AAV nucleotide sequence encoding an ASPA protein comprises a nucleic acid sequence having at least about 90%, at least about 95%, at least about 96%, at least about 97%, at least about 98%, at least about 99%, at least about 99.5%, or 100% identity to SEQ ID NO: 1. In some embodiments, the non-AAV nucleotide sequence (e.g., heterologous sequence) encoding an ASPA protein is the human ASPA complementary DNA (cDNA), optionally linked to a nucleotide sequence encoding a hemagglutinin (HA) tag. In some embodiments, the non-AAV nucleotide sequence (e.g., heterologous sequence) encoding an ASPA protein is linked to a nucleotide sequence encoding a tag, for example hemagglutinin (HA), UA, cMyc, or any suitable tag.

Codon optimization takes advantage of redundancies in the genetic code to enable a nucleotide sequence to be altered while maintaining the same amino acid sequence of the encoded protein. In some embodiments, codon optimization is carried out to facilitate an increase or decrease in the expression of an encoded protein. This is effected by tailoring codon usage in a nucleotide sequence to that of a specific cell type, thus taking advantage of cellular codon bias corresponding to a bias in the relative abundance of particular transfer ribonucleic acids (tRNAs) in the cell type. By altering the codons in the nucleotide sequence so that they are tailored to match the relative abundance of corresponding tRNAs, it is possible to increase expression. Conversely, it is possible to decrease expression by selecting codons for which the corresponding tRNAs are known to be rare in the particular cell type.

In some embodiments, a codon-optimized nucleotide sequence encoding an ASPA protein is more stable than the wild-type cDNA sequence, thereby avoiding generating alternatively spliced variants or truncated proteins if the non-AAV nucleotide sequence is introduced into the transcriptional machinery through gene therapy.

TABLE 1
Non-limiting examples of ASPA and promoter sequences.

| Sequence | | SEQ ID |
|---|---|---|
| description | Sequence | NO |
| Codon | GCCACCATGACAAGCTGCCACATCGCCGAGGAGCACATCCAGAAAGTCGCCATTT | 1 |
| Optimized | TTGGGGGAACTCACGGTAACGAACTCACAGGGGTCTTCCTGGTGAAGCACTGGCT | |
| ASPA gene | CGAGAACGGCGCAGAAATCCAGAGAACCGGACTGGAGGTGAAACCCTTCATTACA | |
| sequence | AATCCTCGGGCCGTCAAGAAATGCACTCGCTACATCGACTGTGATCTGAACCGGA | |
| | TTTTTGATCTGGAAAATCTCGGCAAGAAAATGTCCGAGGACCTGCCATACGAAGT | |
| | GAGGAGAGCTCAGGAGATCAACCACCTCTTCGGACCCAAGGACAGCGAAGATTCC | |
| | TATGACATCATTTTTGATCTGCATAACACCACATCAAATATGGGGTGCACCCTGA | |
| | TCCTCGAGGACAGCCGCAACAATTTCCTGATCCAGATGTTTCACTATATTAAGAC | |
| | AAGTCTGGCACCACTCCCCTGTTACGTGTATCTGATTGAGCATCCCTCTCTCAAG | |
| | TACGCTACTACCCGAAGTATCGCAAAATATCCTGTGGGGATTGAAGTCGGTCCTC | |
| | AGCCACAGGGAGTCCTGCGAGCCGATATCCTCGACCAGATGAGGAAGATGATCAA | |
| | ACATGCTCTGGATTTCATTCACCACTTCAACGAGGGCAAGGAGTTCCCCCCTTGC | |
| | GCCATCGAGGTGTACAAGATCATTGAAAAAGTCGATTATCCTCGGGACGAGAACG | |
| | GCGAAATTGCCGCTATCATTCACCCAAATCTGCAGGACCAGGATTGGAAGCCCCT | |
| | CCATCCTGGGGATCCAATGTTCCTGACACTCGACGGTAAAACTATCCCACTGGGC | |
| | GGAGACTGTACCGTGTACCCCGTGTTTGTCAATGAGGCAGCCTACTATGAGAAGA | |
| | AAGAAGCTTTCGCCAAAACAACAAAACTCACTCTCAATGCTAAATCTATTCGGTG | |
| | CTGCCTCCACTGA | |
| CAG | GCGTTACATAACTTACGGTAAATGGCCCGCCTGGCTGACCGCCCAACGACCCCCG | 2 |
| promoter | CCCATTGACGTCAATAATGACGTATGTTCCCATAGTAACGCCAATAGGGACTTTC | |
| | CATTGACGTCAATGGGTGGAGTATTTACGGTAAACTGCCCACTTGGCAGTACATC | |
| | AAGTGTATCATATGCCAAGTACGCCCCCTATTGACGTCAATGACGGTAAATGGCC | |
| | CGCCTGGCATTATGCCCAGTACATGACCTTATGGGACTTTCCTACTTGGCAGTAC | |
| | ATCTACGTATTAGTCATCGCTATTACCATGGTCGAGGTGAGCCCCACGTTCTGCT | |
| | TCACTCTCCCCATCTCCCCCCCCTCCCCACCCCCAATTTTGTATTTATTTATTTT | |
| | TTAATTATTTTGTGCAGCGATGGGGGCGGGGGGGGGGGGGGGGCGCGCGCCAGGC | |
| | GGGGCGGGGCGGGGCGAGGGGCGGGGCGGGGCGAGGCGGAGAGGTGCGGCGGCAG | |
| | CCAATCAGAGCGGCGCGCTCCGAAAGTTTCCTTTTATGGCGAGGCGGCGGCGGCG | |
| | GCGGCCCTATAAAAAGCGAAGCGCGCGGCGGGCG | |
| PGK promoter | CCGGTAGGCGCCAACCGGCTCCGTTCTTTGGTGGCCCCTTCGCGCCACCTTCTAC | 3 |
| | TCCTCCCCTAGTCAGGAAGTTCCCCCCCGCCCCGCAGCTCGCGTCGTGCAGGACG | |
| | TGACAAATGGAAGTAGCACGTCTCACTAGTCTCGTGCAGATGGACAGCACCGCTG | |
| | AGCAATGGAAGCGGGTAGGCCTTTGGGGCAGCGGCCAATAGCAGCTTTGCTCCTT | |
| | CGCTTTCTGGGCTCAGAGGCTGGGAAGGGGTGGGTCCGGGGGCGGGCTCAGGGGC | |
| | GGGCTCAGGGGCGGGGGGGGCGCCCGAAGGTCCTCCGGAGGCCCGGCATTCTGCA | |
| | CGCTTCAAAAGCGCACGTCTGCCGCGCTGTTCTCCTCTTCCTCATCTCCGGGCCT | |
| | TTCGACCTGCAGCC | |
| CB6 Promoter | CCACGTTCTGCTTCACTCTCCCCATCTCCCCCCCCTCCCCACCCCCAATTTTGTA | 4 |
| | TTTATTTATTTTTTAATTATTTTGTGCAGCGATGGGGGCGGGGGGGGGGGGCGCG | |
| | CGCCAGGCGGGGCGGGGCGGGGCGAGGGGCGGGGCGGGGCGAGGCGGAGAGGTGC | |
| | GGCGGCAGCCAATCAGAGCGGCGCGCTCCGAAAGTTTCCTTTTATGGCGAGGCGG | |
| | CGGCGGCGGCGGCCCTATAAAAAGCGAAGCGCGCGGCGGG | |
| CBA | TGGTCGAGGTGAGCCCCACGTTCTGCTTCACTCTCCCCATCTCCCCCCCCTCCCC | 5 |
| Promoter | ACCCCCAATTTTGTATTTATTTATTTTTTAATTATTTTGTGCAGCGATGGGGGCG | |
| | GGGGGGGGGGGGGGGCGCGCGCCAGGCGGGGGGGGGCGGGGCGAGGGGCGGGGCG | |
| | GGGCGAGGCGGAGAGGTGCGGCGGCAGCCAATCAGAGCGGCGCGCTCCGAAAGTT | |
| | TCCTTTTATGGCGAGGCGGCGGCGGCGGCGGCCCTATAAAAAGCGAAGCGCGCGG | |
| | CGGGCGG | |
| ASPA protein | MTSCHIAEEHIQKVAIFGGTHGNELTGVFLVKHWLENGAEIQRTGLEVKPFITNP | 6 |
| | RAVKKCTRYIDCDLNRIFDLENLGKKMSEDLPYEVRRAQEINHLFGPKDSEDSYD | |
| | IIFDLHNTTSNMGCTLILEDSRNNFLIQMFHYIKTSLAPLPCYVYLIEHPSLKYA | |
| | TTRSIAKYPVGIEVGPQPQGVLRADILDQMRKMIKHALDFIHHFNEGKEFPPCAI | |
| | EVYKIIEKVDYPRDENGEIAAIIHPNLQDQDWKPLHPGDPMFLTLDGKTIPLGGD | |
| | CTVYPVFVNEAAYYEKKEAFAKTTKLTLNAKSIRCCLH | |

The terms “homologous” or “homology,” mean that two or more referenced entities are the same over a defined area (e.g., region, domain, or portion) (e.g., when the entities are aligned). An “aligned” sequence refers to multiple polynucleotide or protein (amino acid) sequences, often containing corrections for missing or additional bases or amino acids (gaps) as compared to a reference sequence. When two sequences are at least partially homologous, they share at least partial identity. “Areas,” “regions” or “domains” of homology or identity mean that a portion of two or more referenced entities are the same, such that they share homology or identity. Thus, where two sequences are identical over one or more sequence regions they share identity in these regions. By way of example, when two polypeptide sequences are identical, they have the same amino acid sequence, at least within the referenced region or portion. Similarly, where two polynucleotide sequences are identical, they have the same polynucleotide sequence, at least within the referenced region or portion. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

The identity or homology between two sequences can extend over the entire sequence length or a portion of the sequence. In some embodiments, the length of the sequence sharing the percent identity is 2, 3, 4, 5, or more contiguous polynucleotide or amino acids, e.g., at least 6, 7, 8, 9, 10, 11, 12, 13, 14, 15, 16, 17, 18, 19, 20, or more contiguous amino acids. In some embodiments, the length of the sequence sharing identity is 20 or more contiguous polynucleotide or amino acids, e.g., at least 20, 21, 22, 23, 24, 25, 26, 27, 28, 29, 30, 31, 32, 33, 34, 35, or more contiguous amino acids. In some embodiments, the length of the sequence sharing identity is 35 or more contiguous polynucleotide or amino acids, e.g., at least 35, 36, 37, 38, 39, 40, 41, 42, 43, 44, 45, 46, 47, 48, 49, 50, or more contiguous amino acids. In some embodiments, the length of the sequence sharing identity is 50 or more contiguous polynucleotide or amino acids, e.g., at least 50-55, 55-60, 60-65, 65-70, 70-75, 75-80, 80-85, 85-90, 90-95, 95-100, 100-110, or more contiguous polynucleotide or amino acids.

The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, for example, Sievers et al., “Fast, scalable generation of high-quality protein multiple sequence alignments using Clustal Omega.” (2011 October 11) Molecular systems biology 7:539. For the purposes of calculating identity to a sequence, extensions such as tags are not included.

Vector genome sequences, including rAAV vector genome sequences described herein, can include one or more “expression control elements.” Typically, expression control elements are nucleic acid sequences that influence expression of an operably linked polynucleotide.

Control elements, including expression control elements as set forth herein, such as promoters and enhancers, present within a vector are included to facilitate proper heterologous polynucleotide (e.g., ASPA gene) transcription and/or translation (e.g., a promoter, enhancer, splicing signal for introns, maintenance of the correct reading frame of the gene to permit in-frame translation of mRNA, etc.). Expression control elements include appropriate transcription initiation, termination, promoter, and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and, in some cases, sequences that enhance secretion of the encoded product (e.g., ASPA). In some embodiments, an rAAV vector genome sequence of the present disclosure comprises a consensus sequence such as a Kozak sequence (for example, a DNA sequence transcribed to an RNA Kozak sequence). In some embodiments, an rAAV vector genome sequence of the present disclosure comprises a Kozak sequence upstream of the nucleotide sequence encoding an ASPA protein. In some embodiments, an RNA Kozak sequence comprises or consists of ACCAUGG (SEQ ID NO:44), GCCGCCACCAUGG (SEQ ID NO:45), CCACCAUG (SEQ ID NO:46), or CCACCAUGG (SEQ ID NO:47).

Expression control can be carried out at the level of transcription, translation, splicing, message stability, etc. Typically, an expression control element that modulates transcription is juxtaposed near the 5′ end of the transcribed polynucleotide (i.e., “upstream”). Expression control elements can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, 5000 to 10,000, or more nucleotides from the nucleotide sequence expressing ASPA), even at considerable distances. Nevertheless, owing to the polynucleotide length limitations, for AAV vectors, such expression control elements will typically be within 1 to 1000 nucleotides from the nucleotide sequence encoding ASPA.

Functionally, expression of an operably linked nucleotide sequence encoding ASPA is at least in part controllable by the element (e.g., promoter) such that the element modulates transcription of the nucleotide sequence and, as appropriate, translation of the transcript. A specific example of an expression control element is a promoter, which is usually located 5′ of the transcribed sequence. Another example of an expression control element is an enhancer, which can be located 5′ of the transcribed sequence, 3′ of the transcribed sequence, or within the transcribed sequence.

A “promoter” as used herein can refer to a nucleic acid sequence that is located adjacent to a nucleic acid sequence (e.g., heterologous polynucleotide) that encodes a recombinant product (e.g., ASPA). A promoter is typically operatively linked to an adjacent sequence, e.g., heterologous polynucleotide. A promoter typically increases an amount expressed from a heterologous polynucleotide as compared to an amount expressed when no promoter exists.

An “enhancer” as used herein can refer to a sequence that is located adjacent to a nucleotide sequence encoding ASPA. Enhancer elements are typically located upstream of a promoter element but also function and can be located downstream of or within a DNA sequence (e.g., a nucleotide sequence encoding ASPA). Hence, an enhancer element can be located 100 base pairs, 200 base pairs, or 300 or more base pairs upstream or downstream of a heterologous polynucleotide. Enhancer elements typically increase expression of a heterologous polynucleotide above the level of increased expression afforded by a promoter element.

In some embodiments, expression control elements include ubiquitous, constitutive or promiscuous promoters and/or enhancers which are capable of driving expression of a polynucleotide in many different cell types. Such elements include, but are not limited to, a cytomegalovirus/0-actin hybrid (e.g., CAG, CB6, or CBA) promoter, a phosphoglycerol kinase (PGK) promoter, cytomegalovirus (CMV) immediate early promoter and/or enhancer sequences, the Rous sarcoma virus (RSV) promoter and/or enhancer sequences and other viral promoters and/or enhancers active in a variety of mammalian cell types, or synthetic elements that are not present in nature (see, e.g., Boshart et al, Cell, 41:521-530 (1985)), the SV40 promoter, the dihydrofolate reductase promoter, the chicken R-actin (CBA) promoter, the EF 1 promoter (Invitrogen), the immediate early CMV enhancer coupled with the CBA promoter (Beltran et al., Gene Therapy, 17(9): 1162-1174 (2010)), and the CBh promoter (Gray et al., Hum Gene Ther, 22(9): 1143-1153 (2011)). In some embodiments, an rAAV of the present disclosure comprises a synthetic CASI promoter which contains a portion of the CMV enhancer, a portion of the chicken beta-actin promoter, and a portion of the UBC enhancer. (See, e.g., International Patent Publication No. WO2012115980. In some embodiments, the promoter is an astrocyte-specific promoter, a glial fibrillary acidic protein (GFAP) promoter, or an enhanced chicken R-actin promoter. In some embodiments, the promoter is a cytomegalovirus/0-actin hybrid promoter, or a PGK promoter. In some embodiments, the cytomegalovirus/0-actin hybrid promoter is a CAG promoter, a CB6 promoter, or a CBA promoter.

In some embodiments, an rAAV vector comprises a CAG promoter sequence comprising SEQ ID NO:2 or a nucleotide sequence having at least about 90%, at least about 91%, at least about 92%, at least about 93%, at least about 94%, at least about 95%, at least about 96%, at least about 97%, at least about 98% or at least about 99%, or 100% identity to SEQ ID NO:2. In some embodiments, an rAAV vector comprises a PGK promoter sequence comprising SEQ ID NO:3 or a nucleotide sequence with at least about 90%, at least about 91%, at least about 92%, at least about 93%, at least about 94%, at least about 95%, at least about 96%, at least about 97%, at least about 98% or at least about 99%, or 100% identity to SEQ ID NO:3. In some embodiments, an rAAV vector comprises a CB6 promoter sequence comprising SEQ ID NO:4 or a nucleotide sequence with at least about 90%, at least about 91%, at least about 92%, at least about 93%, at least about 94%, at least about 95%, at least about 96%, at least about 97%, at least about 98% or at least about 99%, or 100% identity to SEQ ID NO:4.

In some embodiments, an rAAV vector comprises a CBA promoter sequence comprising SEQ ID NO:5 or a nucleotide sequence having at least about 90%, at least about 91%, at least about 92%, at least about 93%, at least about 94%, at least about 95%, at least about 96%, at least about 97%, at least about 98% or at least about 99%, or 100% identity to SEQ ID NO:5. Table 1 includes non-limiting examples of promoter sequences.

Inducible promoters allow regulation of gene expression and can be regulated by exogenously supplied compounds, environmental factors such as temperature, or the presence of a specific physiological state, acute phase, a particular differentiation state of the cell, or in replicating cells only. Inducible promoters and inducible systems are available from a variety of commercial sources, including, without limitation, Invitrogen and Clontech. Many other systems have been described and are available for use. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system, and the rapamycin-inducible system. Any type of inducible promoter which is tightly regulated and is specific for the particular target cell type in which ASPA expression is intended may be used.

Expression control elements (e.g., promoters) include those active in a particular tissue or cell type, referred to herein as “tissue-specific expression control elements/promoters.” Tissue-specific expression control elements are typically active in specific cell or tissue (e.g., brain, central nervous system, spinal cord, etc.). Expression control elements are typically active in these cells, tissues, or organs because they are recognized by transcriptional activator proteins, or other regulators of transcription, that are unique to a specific cell, tissue, or organ type. Thus, in some cases, an rAAV vector of the present disclosure comprises a promoter that directs expression of the nucleotide sequence encoding ASPA protein in a host cell (e.g., a nerve cell, such as, an oligodendrocyte, Schwann cell, microglial cell, astrocyte or neuron; or a non-nerve cell, such as a hepatocyte).

In some embodiments, the regulatory sequences useful in the rAAV vectors of the present disclosure also contain an intron, which intron is optionally located between the promoter/enhancer sequence and the ASPA gene. In some embodiments, the intron sequence is derived from SV-40, and is a 100 bp mini-intron splice donor/splice acceptor referred to as SD-SA. In some embodiments, an rAAV vector comprises a posttranscriptional regulatory element. One example of a posttranscriptional regulatory element is the woodchuck hepatitis virus post-transcriptional element (WPRE). (See, e.g., Wang and Verma, Proc. Natl. Acad. Sci., USA, 96: 3906-3910 (1999)). In certain embodiments, a posttranscriptional regulatory element is a hepatitis B virus posttranscriptional regulatory element (HBVPRE) or a RNA transport element (RTE). In some embodiments, the WPRE or HBVPRE sequence is any of the WPRE or HBVPRE sequences disclosed in U.S. Pat. No. 6,136,597 or U.S. Pat. No. 6,287,814. In some embodiments, a WPRE sequence comprises or consists of:

(SEQ ID NO: 51)
aatcaacctc tggattacaa aatttgtgaa agattgactg
atattcttaa ctatgttgct ccttttacgc tgtgtggata
tgctgcttta atgcctctgt atcatgctat tgcttcccgt
acggctttcg ttttctcctc cttgtataaa tcctggttgc
tgtctcttta tgaggagttg tggcccgttg tccgtcaacg
tggcgtggtg tgctctgtgt ttgctgacgc aacccccact
ggctggggca ttgccaccac ctgtcaactc ctttctggga
ctttcgcttt ccccctcccg atcgccacgg cagaactcat
cgccgcctgc cttgcccgct gctggacagg ggctaggttg
ctgggcactg ataattccgt ggtgttgtcg gggaagctga
cgtcctttcc atggctgctc gcctgtgttg ccaactggat
cctgcgcggg acgtccttct gctacgtccc ttcggctctc
aatccagcgg acctcccttc ccgaggcctt ctgccggttc
tgcggcctct cccgcgtctt cgctttcggc ctccgacgag
tcggatctcc ctttgggccg cctccccgcc tg.

In some embodiments, an rAAV vector comprises a polyA signal. PolyA signals may be derived from many suitable species, including, without limitation, rabbit, SV-40, human, and bovine. In some embodiments, an rAAV vector comprises a rabbit globin polyA signal, such as, a rabbit β-globin polyA signal. In some embodiments, the rabbit β-globin polyA signal has a nucleic acid sequence of SEQ ID NO: 101 or a nucleotide sequence having at least about 90%, at least about 91%, at least about 92%, at least about 93%, at least about 94%, at least about 95%, at least about 96%, at least about 97%, at least about 98% or at least about 99%, or 100% identical to SEQ ID NO: 101 (SEQ ID NO: 101-aataaaggaa atttattttc attgcaatag tgtgttggaa ttttttgtgt ctctca).

Another useful regulatory component that may be included in an rAAV vector is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence. The IRES may be located 5′ or 3′ to the ASPA transgene in the rAAV vector. In other embodiments, an rAAV vector may comprise a nucleotide sequence encoding a 2A peptide that allows for expression of multiple polypeptides from a single promoter.

As used herein, a recombinant “vector” or “rAAV vector” refers to a vector that is derived from the wild type genome of a virus such as AAV by using molecular methods to remove the wild type genome from the virus, and replace it with a non-native nucleic acid, such as a heterologous polynucleotide sequence (e.g., a therapeutic gene expression cassette expressing ASPA). Typically, for AAV, one or both inverted terminal repeat (ITR) sequences of the wild-type AAV genome are retained in the AAV vector. An rAAV vector can be distinguished from a viral genome, because all (or a part) of the viral genome has been replaced with a non-native sequence with respect to the viral genomic nucleic acid. Incorporation of a non-native sequence such as a heterologous polynucleotide therefore defines the viral vector as a “recombinant” vector, which in the case of AAV can be referred to as an “rAAV vector.” An rAAV vector comprising a nucleic acid molecule encoding ASPA may also be referred to as a “scAAV9-CB6-hASPAopt” or an “ASPA vector”. As will be apparent from context, “vector” may refer to an isolated recombinant nucleotide sequence or an AAV particle or virion comprising a recombinant nucleotide sequence.

In some embodiments, an rAAV vector does not comprise any binding sites for miRNA (microRNA). In some embodiments, an rAAV vector comprises one, two, three, four, five, or more binding sites for an miRNA that is expressed in cells where expression of the ASPA protein is not desired (i.e., de-targeting). In some embodiments, an rAAV vector comprises one or more binding sites for miR-122. Binding of miR-122 to the ASPA-encoding sequence may reduce expression of this sequence in liver cells, where miR-122 is highly prevalent (Thakral and Ghoshal, Curr Gene Ther. 2015; 15(2): 142-150).

An rAAV nucleic acid sequence can be packaged into a virus (also referred to herein as a “particle” or “virion”) for subsequent infection (transformation) of a cell, ex vivo, in vitro, or in vivo. Where a recombinant vector sequence is encapsidated or packaged into an AAV particle, the particle can be referred to as an “rAAV.” Such particles or virions will typically include proteins that encapsidate or package the vector genome. Particular examples include viral envelope proteins, and, in the case of AAV, capsid proteins.

The AAV components of the rAAV vectors and particles described herein may be selected from various AAV serotypes. In some embodiments, an rAAV vector comprises an AAV nucleic acid sequence from an rh10, AAV1, AAV2, AAV3, AAV4, AAV5, AAV6, AAV7, AAV8, AAV9, AAV10, AAV11, AAV12, orrh74 serotype. In some embodiments, an rAAV vector comprises an AAV nucleic acid sequence from an AAV9 serotype. These AAV components may be readily isolated using various techniques from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank™, PubMed, or the like.

In certain embodiments, an rAAV vector or rAAV particle comprises an AAV nucleic acid sequence or AAV protein as disclosed in, e.g., U.S. Pat. No. 7,906,111 or U.S. Pat. No. 7,629,322, which are herein incorporated by reference in their entireties. In some embodiments, an rAAV vector or rAAV particle comprises an AAV nucleic acid sequence or AAV protein from AAV serotype AAV8 or its variants, as disclosed in, e.g., U.S. Pat. Nos. 7,282,199, 9,587,250 or 9,677,089, which are herein incorporated by reference in their entireties. In some embodiments, an rAAV vector or rAAV particle comprises an AAV nucleic acid sequence or AAV protein from AAV serotype AAV9 or its variants, as disclosed in, e.g., U.S. Pat. No. 7,198,951, which is herein incorporated by reference in its entirety. In some embodiments, an rAAV vector or rAAV particle comprises an AAV nucleic acid sequence or AAV protein from AAV serotype rh74 or its variants.

In some embodiments, an rAAV vector of the present disclosure comprises a nucleic acid molecule comprising at least one AAV ITR sequence. In certain embodiments, an rAAV vector comprises two ITR sequences, which ITR sequences may be of the same or different AAV serotypes. AAV ITRs may be selected from among any useful AAV serotype, including, without limitation, rh10, AAV1, AAV2, AAV3, AAV4, AAV5, AAV6, AAV7, AAV8, AAV9, AAV10, AAV11, AAV12, rh74, and other AAV serotypes. In some embodiments, an rAAV vector described herein comprises a genome comprising a sequence of one or two AAV2 ITRs.

The present disclosure further provides an rAAV particle comprising an rAAV vector described herein. Thus, in some aspects, the present disclosure relates to an rAAV particle comprising a nucleic acid molecule comprising at least one AAV ITR and a non-AAV nucleotide sequence (also referred to as a heterologous polynucleotide) encoding an ASPA protein, the non-AAV nucleotide sequence operably linked to a promoter. In some embodiments, an rAAV particle comprises at least one capsid protein selected from the group consisting of AAV serotype rh10, AAV1, AAV2, AAV3, AAV4, AAV5, AAV6, AAV7, AAV8, AAV9, AAV10, AAV11, AAV12, rh74, and other AAV serotypes. In some embodiments, an rAAV particle comprises at least one AAV9 capsid protein.

In some embodiments, an rAAV vector or particle comprises an AAV9 capsid protein, and a nucleic acid molecule comprising human ASPA cDNA; a CAG, PGK, CBA or CB6 promoter; and, optionally, one or two AAV2 ITR sequences. In some embodiments, an rAAV vector or particle is an AAV9-CAG-hASPAopt, AAV9-PGK-hASPAopt, AAV9-CBA-hASPAopt or AAV9-CB6-hASPAopt vector. In some embodiments, a promoter comprises or consists of SEQ ID NO:2, SEQ ID NO:3, SEQ ID NO:4, or SEQ ID NO:5 or a nucleotide sequence having at least about 90%, at least about 91%, at least about 92%, at least about 93%, at least about 94%, at least about 95%, at least about 96%, at least about 97%, at least about 98%, or at least about 99% identity to SEQ ID NO:2, SEQ ID NO:3, SEQ ID NO:4, or SEQ ID NO:5. In some embodiments, an rAAV vector comprises a Kozak sequence. In some embodiments, a Kozak sequence comprises or is capable of being transcribed to SEQ ID NO:44, SEQ ID NO:45, SEQ ID NO:46, or SEQ ID NO:47. In some embodiments, an rAAV vector further comprises an HBVPRE sequence or a WPRE sequence (e.g., SEQ ID NO:51).

In some embodiments, an rAAV vector or rAAV particle comprises a capsid (“Cap”) protein (e.g., including the vp1, vp2, vp3 and hypervariable regions), a viral replication (“Rep”) protein (e.g., rep 78, rep 68, rep 52, or rep 40), and/or a sequence encoding one or more such proteins. These AAV components may be readily utilized in a variety of vector systems and host cells. Such components may be used alone, in combination with other AAV serotype sequences or components, or in combination with elements from non-AAV viral sequences.

As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid.

Pseudotyped vectors, wherein the capsid of one AAV is replaced with a heterologous capsid protein, are useful in the present disclosure. In some embodiments, the AAV is AAV2/5. In some embodiments, the AAV is AAV2/8. See, e.g., Mussolino et al., Gene Therapy, 18(7): 637-645 (2011); Rabinowitz et al., J Virol, 76(2): 791-801 (2002).

In some embodiments, vectors useful in compositions and methods described herein contain, at a minimum, sequences encoding a selected AAV serotype capsid, or a fragment thereof. In some embodiments, useful vectors contain, at a minimum, sequences encoding a selected AAV serotype rep protein, or a fragment thereof. Optionally, such vectors may contain both AAV cap and rep proteins. In vectors in which both AAV rep and cap are provided, the AAV rep and AAV cap sequences can both be of one serotype. Alternatively, vectors may be used in which the rep sequences are from one AAV serotype and the cap sequences are from a different AAV serotype. In some embodiments, the rep and cap sequences are expressed from separate sources (e.g., separate vectors, or a host cell and a vector). In some embodiments, these rep sequences are fused in frame to cap sequences of a different AAV serotype to form a chimeric AAV vector, such as AAV2/8 described in U.S. Pat. No. 7,282,199, which is herein incorporated by reference in its entirety.

A suitable rAAV can be generated by culturing a host cell which contains a nucleic acid sequence encoding an AAV serotype capsid protein, or fragment thereof, as defined herein; a functional rep gene; a nucleic acid molecule composed of, at a minimum, AAV inverted terminal repeats (ITRs) and an ASPA-encoding nucleic acid sequence; and sufficient helper functions to permit packaging of the nucleic acid molecule into the AAV capsid protein. In some aspects, the present disclosure provides a host cell comprising an rAAV vector or an rAAV particle disclosed herein. The components required to be present in the host cell to package an rAAV vector in an AAV capsid may be provided to the host cell in trans.

Alternatively, any one or more of the required components (e.g., vector, rep sequences, cap sequences, and/or helper functions) may be provided by a stable host cell which has been engineered to contain one or more of the required components using various methods. Most suitably, such a stable host cell will contain the required component(s) under the control of an inducible promoter. However, the required component(s) may be under the control of a constitutive promoter. Examples of suitable inducible and constitutive promoters are provided herein, in the discussion above of regulatory elements suitable for use with a non-AAV nucleotide sequence, i.e., ASPA. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated.

The rAAV vector, rep sequences, cap sequences, and helper functions used to produce the rAAV of the present disclosure may be delivered to the packaging host cell in the form of any genetic element which transfers the sequences carried thereon. The selected genetic element may be delivered by any suitable method, including those described herein. The methods used to construct any embodiment of this disclosure, including generating rAAV particles, are known to those with skill in nucleic acid manipulation and include genetic engineering, recombinant engineering and synthetic techniques. See, e.g., Sambrook et al, Molecular Cloning: A Laboratory Manual, Cold Spring Harbor Press, Cold Spring Harbor, N.Y.; Fisher et al, J. Virol., 70:520-532 (1993); and U.S. Pat. No. 5,478,745, each of which are herein incorporated by reference in their entireties.

In an aspect, the present disclosure provides a method of producing an rAAV particle, the method comprising culturing a host cell containing: (a) a nucleic acid molecule comprising or consisting of an rAAV vector genome expressing ASPA as described herein; (b) a nucleic acid molecule encoding an AAV rep; (c) a nucleic acid molecule encoding at least one AAV capsid protein; and (d) sufficient helper functions for packaging the rAAV vector genome into the rAAV particle.

rAAV particles of the present disclosure may be purified by various methods. In some embodiments, an rAAV virus may be purified by anion exchange chromatography. See, e.g., U.S. Patent Publication No. 2018/0163183 A1. Further details regarding the construction and characterization of the AAV vectors and particles disclosed herein is described in International Patent Publication No. WO 2019/143803, which is incorporated herein by reference in its entirety. Further details on rAAV particles encoding ASPA may be found at U.S. Pat. No. 9,102,949, U.S. Patent Publication No: 2019/0125899A1, and U.S. Patent Publication No: US 2018/0311323A1, the contents of each of which are herein incorporated by reference in their entireties.

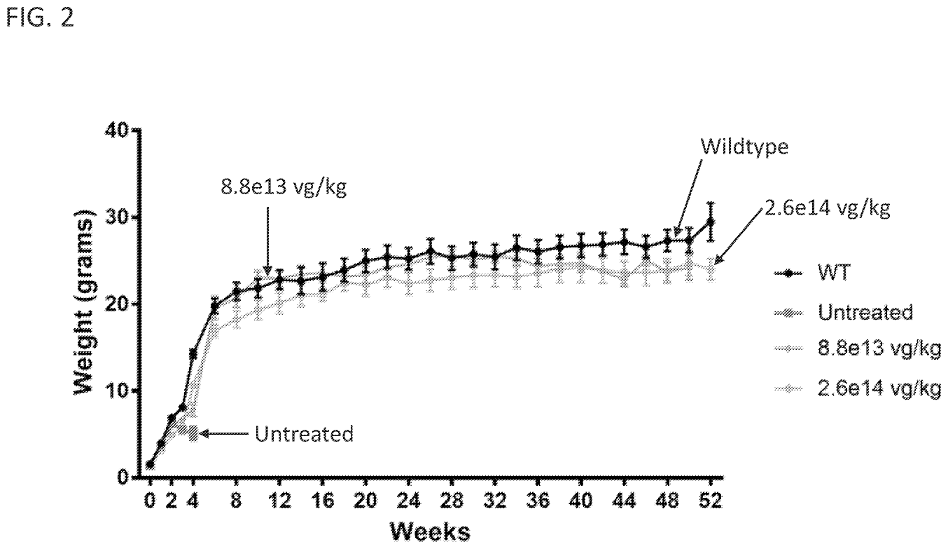

In some embodiments, the composition comprising the non-replicating, recombinant AAV serotype 9 (AAV9) vector containing an expression cassette for the human ASPA transgene disclosed herein has the AAV-hASPAopt-Opt vector design depicted in FIG. 2. In some embodiments, the expression cassette of the rAAV vector comprises a 5′ ITR comprising the nucleic acid sequence of SEQ ID NO: 102 (SEQ ID NO: 102-ctgcgcgctc gctcgctcac tgaggccggg cgaccaaagg tcgcccgacg cccgggcttt gcccgggcgg cctcagtgag cgagcgagcg cgcagagagg gagtggccaa ctccatcact aggggttcct). In some embodiments, the expression cassette of the rAAV vector comprises a codon optimized nucleic acid sequence encoding hASPA of SEQ ID NO: 1. In some embodiments, the expression cassette of the rAAV vector comprises a rabbit β-globin polyA comprising the nucleic acid sequence of SEQ ID NO: 101. In some embodiments, the expression cassette of the rAAV vector comprises a 3′ ITR comprising the nucleic acid sequence of SEQ ID NO: 103 (SEQ ID NO: 103-aggaacccct agtgatggag ttggccactc cctctctgcg cgctcgctcg ctcactgagg ccgggcgacc aaaggtcgcc cgacgcccgg gctttgcccg ggcggcctca gtgagcgagc gagcgcgcag).

### Pharmaceutical Compositions

The rAAV vectors or particles of the present disclosure can be incorporated into pharmaceutical compositions suitable for administration. In an aspect, the present disclosure provides a pharmaceutical composition comprising an rAAV vector or an rAAV particle disclosed herein (e.g., an rAAV particle comprising a nucleic acid sequence encoding ASPA) and a pharmaceutically acceptable carrier, diluent, or excipient. As used herein, the term “pharmaceutically acceptable” refers to molecular entities and compositions that do not generally produce allergic or other serious adverse reactions when administered using established routes. Molecular entities and compositions approved by a regulatory agency of the U.S. Federal or a U.S. state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, buffered saline, Ringer's solutions, dextrose solution, 5% human serum albumin, and other buffers, e.g., HEPES, to maintain pH at appropriate physiological levels. Except insofar as any media or agent is incompatible with the active component, use thereof in the compositions is contemplated. Supplementary active compounds can also be incorporated into the compositions.

Throughout this description, “vg” may refer to “viral genomes” or “vector genomes.”

Examples of pharmaceutical compositions and delivery systems that may be used for administration of the rAAV disclosed herein can be found in Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technomic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315.

A pharmaceutical composition of the present disclosure may be formulated to be compatible with its intended route of administration. Examples of routes of administration include parenteral administration, e.g., intravenous administration, or injection. Solutions or suspensions used for parenteral (e.g., intravenous or via injection) application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition should be sterile and should be fluid to the extent that easy syringeability exists. The composition should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof.

The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents such as sugars, polyalcohols (e.g., polyols) such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional ingredient from a previously sterile-filtered solution thereof.

For injection, a pharmaceutically acceptable carrier can be a liquid. Exemplary physiologically acceptable carriers include sterile, pyrogen-free water and sterile, pyrogen-free, phosphate buffered saline. A variety of such known carriers are provided in U.S. Pat. No. 7,629,322, which is herein incorporated by reference in its entirety. In some embodiments, the carrier is an isotonic sodium chloride solution. In some embodiments, the carrier is balanced salt solution. In some embodiments, the carrier includes TWEEN® (polysorbate). If the rAAV is to be stored long-term, it may be frozen in the presence of glycerol or TWEEN® (polysorbate) 20.

It is especially advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of active agent (e.g., rAAV) calculated to produce the desired therapeutic effect in association with the required pharmaceutical carrier. The specification for the dosage unit forms of the present disclosure are dictated by and directly dependent on the unique characteristics of the active agent (e.g., rAAV) and the particular therapeutic effect to be achieved, and the limitations inherent in the art of compounding such an active agent (e.g., rAAV) for the treatment of individuals. Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Recombinant vector (e.g., rAAV) sequences, plasmids, vector genomes, recombinant virus particles (e.g., rAAV), and pharmaceutical compositions thereof can be packaged in single or multiple unit dosage form for ease of administration and uniformity of dosage.

A pharmaceutical composition comprising an rAAV vector or an rAAV particle comprising a nucleic acid sequence encoding ASPA can be included in a container, pack, or dispenser together with instructions for administration.

In some embodiments, the composition includes a surfactant. In some embodiments, the composition includes a poloxamer such as poloxamer 188. In some embodiments, the composition includes a thickener, plasticizer, and/or cryoprotectant. In some embodiments, the composition includes a sugar alcohol such as sorbitol. In some embodiments, the composition includes a buffer. In some embodiments, the composition is formulated as a sterile solution in phosphate buffered saline, pH 7.0, 5% weight/volume (w/v) sorbitol, 0.001% w/v poloxamer 188. It may be supplied in 5-milliliter (mL) pyrogen-free vials at a fill volume of 2.5 mL and sealed with a latex-free rubber stopper and aluminum flip-off seal. In some embodiments, the composition is formulated as a sterile solution comprising 137 millimolar (mM) sodium chloride, 8.1 mM sodium phosphate dibasic, 1.47 mM potassium dihydrogen phosphate, 2.7 mM potassium chloride, 5% w/v sorbitol, 0.001% w/v poloxamer 188, and Water for Injection. The vector concentration of the formulated product is not limited, and may be in the range of about 10¹² vg/mL to about 10¹⁵ vg/mL, for example, about 5×10¹² vg/mL, about 1×10¹³ vg/mL, about 5×10¹³ vg/mL, about 1×10¹⁴ vg/mL, about 5×10¹⁴ vg/mL, including all values and subranges that lie therebetween. In some embodiments, the vector concentration of the formulated product is in the range of about 1.8×10¹³ vg/mL to about 5.0×10¹³ vg/mL. In some embodiments, the vector concentration of the formulated product is in the range of about 1.8×10¹³ vg/mL to about 4.2×10¹³ vg/mL. The vials may be stored frozen at <−60° C. until ready for use.

In an aspect, the present disclosure provides a kit comprising an rAAV vector, or a particle comprising the same, where the rAAV vector comprises a nucleic acid molecule comprising at least one AAV inverted terminal repeat (ITR) and a non-AAV nucleotide sequence encoding a ASPA protein, where the non-AAV nucleotide sequence is operably linked to a promoter. In some embodiments, the kit further comprises instructions for administering the rAAV vector to a subject. In some embodiments, the kit further comprises instructions for administering the rAAV vector by intravenous infusion.

In another aspect, the present disclosure provides a unit dose comprising an rAAV vector, or a particle comprising the same, where the rAAV vector comprises a nucleic acid molecule comprising at least one AAV inverted terminal repeat (ITR) and a non-AAV nucleotide sequence encoding an ASPA protein, where the non-AAV nucleotide sequence is operably linked to a promoter; and where the therapeutically effective amount is in the range of about 10¹³ vg/kg to about 10¹⁵ vg/kg. In some embodiments, the therapeutically effective amount is about 1.32×10¹⁴ vg/kg. In some embodiments, the therapeutically effective amount is about 3×10¹⁴ vg/kg. In some embodiments, the unit dose comprises a liquid formulation.

In some embodiments, the unit dose is configured for administration to a subject by intravenous infusion.

In some embodiments of the above aspects, the ASPA protein is human ASPA protein. In some embodiments of the above aspects, the non-AAV nucleotide sequence encoding an ASPA protein comprises or consists of the human ASPA cDNA. In some embodiments of the above aspects, the non-AAV nucleotide sequence encoding an ASPA protein comprises or consists of a codon-optimized nucleotide sequence. In some embodiments of the above aspects, the non-AAV nucleotide sequence encoding an ASPA protein comprises or consists of SEQ ID NO: 1. In some embodiments of the above aspects, the non-AAV nucleotide sequence encoding an ASPA protein encodes the amino acid sequence of SEQ ID NO: 1 or an amino acid sequence at least about 95%, at least about 96%, at least about 97%, at least about 98% or at least about 99% identical to SEQ ID NO:1. In some embodiments of the above aspects, the promoter directs expression of the ASPA protein in a host cell, such as, a nerve cell. In some embodiments of the above aspects, the promoter is a cytomegalovirus/0-actin hybrid promoter, or a PGK promoter. In some embodiments of the above aspects, the cytomegalovirus/0-actin hybrid promoter is a CAG, CB6, or CBA promoter. In some embodiments of the above aspects, the promoter comprises or consists of the nucleotide sequence of SEQ ID NO: 2, SEQ ID NO: 3, SEQ ID NO:4, or SEQ ID NO:5. In some embodiments of the above aspects, the ITR is an AAV1, AAV2, AAV3, AAV4, AAV5, AAV6, AAV7, AAV8, AAV9, AAV10, AAV11, AAV12, rh10, or rh74 serotype ITR. In some embodiments of the above aspects, the rAAV is an AAV1, AAV2, AAV3, AAV4, AAV5, AAV6, AAV7, AAV8, AAV9, AAV10, AAV11, AAV12, rh10, or rh74 serotype. In some embodiments of the above aspects, the rAAV is an AAV9 serotype. In some embodiments of the above aspects, wherein the nucleic acid molecule further comprises a Kozak sequence. In some embodiments of the above aspects, the nucleic acid molecule further comprises an miR-122 binding site.

### Methods of Using Recombinant AAV Vectors and Particles

The present disclosure provides methods involving the use of AAV (e.g., recombinant AAV) vectors, such as in the treatment of a subject in need. The methods provided herein comprise, e.g., administering to a subject a therapeutically effective amount of any one of the rAAVs or compositions disclosed herein. In some embodiments, administration of the rAAV vector results in expression of ASPA in a tissue of the subject. In some embodiments, the tissue is peripheral tissue or central nervous system (CNS) tissue. In some embodiments, administration of the rAAV vector results in expression of ASPA in a cell of the subject. In some embodiments, the cell is a nerve cell, such as, an oligodendrocyte.

In some embodiments, the subject is a human, a non-human primate, a pig, a horse, a cow, a dog, a cat, a rabbit, a guinea pig, a hamster, a mouse, or a rat. In particular embodiments, the subject is human. The human subject may be a human female or a human male. In some embodiments, the subject is a human that has previously undergone a hormone therapy program. In some embodiments, the subject is undergoing a hormone therapy program. In some embodiments, the subject has undergone or is undergoing physical therapy or other physical intervention, such as supportive care or use of a feeding tube. In some embodiments, the subject is undergoing or has undergone treatment for one or more symptoms of Canavan disease, such as anti-seizure or anti-convulsant medication. In some embodiments, the subject has undergone or is undergoing an investigational therapy for Canavan disease, such as lithium gluconate, glyceryl triacetate (GTA), cord blood cell therapy, or an ASPA gene therapy. In some embodiments, the subject is a human infant. In certain cases, the subject is a human infant about 1 month old, about 2 months old, about 3 months old, about 4 months old, about 5 months old, about 6 months old, about 7 months old, about 8 months old, about 9 months old, about 10 months old, about 11 months old, or about 1 year old. In some embodiments, the subject is a human infant less than 3 months old, less than 6 months old, less than 9 months old, less than 1 year old, or less than 18 months old. In some embodiments, the subject is a human who is less than or equal to 30 months old. In some embodiments, the subject is a human child (e.g., <18 years of age), such as a child between 13-18, 12-18, 10-18, 8-18, 6-18, 2-18, 10-13, 8-13, 6-13, 2-13, 10-12, 8-12, 6-12, 2-12, 6-12, 2-12, 2-10, 2-8, or 2-6 years of age, or any range therein. In some embodiments, the subject is a human adult (e.g., >18 years of age).

As used herein, the term “patient in need” or “subject in need” refers to a patient or subject at risk of, or suffering from, a disease, disorder, or condition that is amenable to treatment or amelioration with an rAAV comprising a nucleic acid sequence encoding ASPA or a composition comprising such an rAAV provided herein, such as leukodystrophy. In some embodiments, the “patient or subject in need” is a patient or subject is at risk of developing, or suffers from a disease associated with the malfunction of ASPA, such as, for example, Canavan disease. In some embodiments, the “patient in need” or “subject in need” has one or more amino acid mutations in the ASPA gene resulting in alterations to ASPA function. “Subject” and “patient” are used interchangeably herein.

As used herein, the term “effective amount” or “therapeutically effective amount” refers to the amount of a pharmaceutical agent, e.g., an rAAV, which is sufficient to reduce or ameliorate the severity and/or duration of a disorder, e.g., a leukodystrophy such as Canavan Disease, or one or more symptoms thereof; prevent the advancement of a disorder; cause regression of a disorder; prevent the recurrence, development, onset or progression of one or more symptoms associated with a disorder; detect a disorder; or enhance or improve the prophylactic or therapeutic effect(s) of another therapy (e.g., prophylactic or therapeutic agent).

In some embodiments, the effective amount of an rAAV may, for example, increase the expression of ASPA, and/or relieve to some extent one or more of the symptoms associated with a disease associated with the deficiency of adequate ASPA levels and/or adequate ASPA function.

The disclosure provides methods and uses of the rAAV, comprising a nucleic acid molecule encoding ASPA as described herein for providing a therapeutic benefit to a subject with a disorder or a disease characterized by a deficiency or malfunction of ASPA. In some aspects, a method comprises administering to a subject in need thereof, a therapeutically effective amount of an rAAV or a composition described herein, thereby treating the disorder or disease characterized by a deficiency or malfunction of ASPA in the subject.

In some cases, the present disclosure provides a method of expressing ASPA in a subject in need thereof, comprising administering to the subject a therapeutically effective amount of an rAAV particle described herein or a pharmaceutical composition described herein, thereby expressing ASPA in the subject. In certain embodiments, the present disclosure provides a method of increasing the expression of ASPA in a subject in need thereof, comprising administering to the subject a therapeutically effective amount of an rAAV particle described herein or pharmaceutical composition comprising the particle, thereby increasing expression of ASPA in the subject. In some embodiments, the methods disclosed herein result in the expression of ASPA or increases the expression of ASPA in the nerve cells of the subject.

In some embodiments, the subject is in need of expression of ASPA. In some embodiments, the subject has an ASPA deficiency. The disclosure provides methods of treating a subject with a deficiency of ASPA, comprising administering to the subject a therapeutically effective amount of an rAAV particle described herein or pharmaceutical composition comprising the particle, thereby treating ASPA deficiency in the subject. The disclosure provides methods of treating a subject with a leukodystrophy such as Canavan disease, comprising administering to the subject a therapeutically effective amount of an rAAV particle described herein or pharmaceutical composition comprising the particle, thereby treating the leukodystrophy in the subject.

The leukodystrophy may be any leukodystrophy identified as such by a physician, including the more than 50 types of leukodystrophies that affect myelin function resulting in progressive loss of neurological function. In some embodiments, the leukodystrophy comprises adult-onset autosomal dominant leukodystrophy (ADLD), Aicardi-Goutieres syndrome, Alexander disease, Cerebral Autosomal Dominant Arteriopathy with Sub-cortical Infarcts and Leukoencephalopathy (CADASIL), Canavan disease, Cerebral autosomal recessive arteriopathy with subcortical infarcts and leukoencephalopathy (CARASIL), cerebrotendinous xanthomatosis, childhood ataxia, cerebral hypomyelination (CACH)/vanishing white matter disease (VWMD), Fabry disease, fucosidosis, GM1 gangliosidosis, Krabbe disease, L-2-hydroxyglutaric aciduria, megalencephalic leukoencephalopathy with subcortical cysts, metachromatic leukodystrophy, multiple sulfatase deficiency, Pelizaeus-Merzbacher disease, Pol III-Related Leukodystrophies, Refsum disease, Salla disease (free sialic acid storage disease), Sjogren-Larsson syndrome, X-linked adrenoleukodystrophy, or Zellweger syndrome spectrum disorders.

The disclosure further provides methods of treating, reducing, improving, slowing the progression of, or preventing a symptom of a leukodystrophy (e.g., Canavan disease) in a subject having leukodystrophy, the method comprising administering to the subject a therapeutically effective amount of an rAAV particle described herein or pharmaceutical composition comprising the particle, thereby treating, reducing, improving, slowing the progression of, or preventing a symptom of leukodystrophy in a subject. Non-limiting examples of symptoms of leukodystrophies include problems with one or more of the following: balance, breathing, cognition (learning, thinking, remembering), eating and swallowing, hearing, movement, balance and coordination, speech, and vision.

In some embodiments, the leukodystrophy is Canavan disease. The disclosure provides methods of treating a subject with Canavan disease, comprising administering to the subject a therapeutically effective amount of an rAAV particle described herein or pharmaceutical composition comprising the particle, thereby treating Canavan disease in the subject. In some embodiments, the methods further comprise selecting a subject with Canavan disease prior to administering the rAAV vector. The disclosure further provides methods of treating, reducing, improving, slowing the progression of or preventing a symptom of Canavan disease in a subject having Canavan disease, the method comprising administering to the subject a therapeutically effective amount of an rAAV particle described herein or pharmaceutical composition comprising the particle, thereby treating, reducing, improving, slowing the progression of, or preventing a symptom of Canavan disease in a subject. Non-limiting examples of symptoms of Canavan disease may include one or more of the following: lack of motor development; feeding difficulties; abnormal muscle tone (weakness or stiffness); an abnormally large, poorly controlled head; paralysis, blindness; or hearing loss. In some embodiments, a symptom of Canavan disease is poor head control, macrocephaly (abnormally large head), hypotonia (diminished muscle tone), apathy (unresponsiveness), lethargy, irritability, dysphagia (difficulty swallowing), feeding difficulties, delay in reaching developmental milestones, failure to walk independently, psychomotor regression (progressive loss of abilities and coordination), mental retardation, intellectual disability, seizures, disordered sleeping, nasal regurgitation, reflux sometimes associated with vomiting, deterioration of optic nerves, optic atrophy, reduced visual responsiveness, hearing loss, spasticity, decerebrate rigidity, paralysis, or increased NAA in urine.

In some embodiments, the methods further comprise selecting a subject with leukodystrophy prior to administering the rAAV vector. In some embodiments, the subject may be screened and identified or diagnosed as having leukodystrophy (e.g., by genetic or physiological testing) even though the subject does not have one or more symptoms of the disease. In other embodiments, a subject has one or more symptoms of leukodystrophy. In certain embodiments, a subject has a mutation in a gene associated with a leukodystrophy, such as, Canavan disease. In some embodiments, a subject has a loss-of-function mutation in a gene that controls the expression and/or function of aspartoacylase (ASPA), such as, the gene encoding ASPA. In some embodiments, the subject is diagnosed as having Canavan disease based on a pre-natal blood test that screens for the level of ASPA, and/or for mutations in the ASPA-encoding gene.

In some embodiments, the methods comprise decreasing the level of N-acetylaspartate (NAA) in a biological sample obtained from the subject, as compared to: (a) the level of NAA in the biological sample of the subject prior to the administration of the rAAV particle described herein or pharmaceutical composition comprising the particle, and/or (b) the level of NAA in a biological sample of a control subject, wherein the control subject has leukodystrophy (such as, Canavan disease) and is not administered the rAAV particle described herein or pharmaceutical composition comprising the particle. In some embodiments, the methods comprise decreasing the level of N-acetylaspartate (NAA) in a biological sample obtained from the subject by at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, including all values and subranges that lie therebetween), as compared to: (a) the level of NAA in the biological sample of the subject prior to the administration of the rAAV particle described herein or pharmaceutical composition comprising the particle, and/or (b) the level of NAA in a biological sample of a control subject, wherein the control subject has leukodystrophy (such as, Canavan disease) and is not administered the rAAV particle described herein or pharmaceutical composition comprising the particle. In some embodiments, the biological sample is blood, urine, peripheral tissue, CNS fluid, or CNS tissue.

In some embodiments, it has been determined that there is a metabolic imbalance comprising a shift from glycolysis to beta-oxidation in the subject. In some embodiments, the method further comprises detecting the metabolic imbalance by evaluating levels of one or more glycolysis and/or beta-oxidation factors. In some embodiments, the levels of one or more glycolysis and/or beta-oxidation factors are evaluated using central nervous system (CNS) fluid obtained from the subject. In some embodiments, the method further comprises: (a) obtaining CNS fluid from the subject; (b) detecting increased beta-oxidation in the CNS fluid; and (c) based on the detection in (b), administering the rAAV to the subject. In some embodiments, the method further comprises: (a) measuring a metabolic profile of a biological sample obtained from the subject; and (b) identifying a metabolic imbalance comprising a shift from glycolysis to beta-oxidation based upon the metabolic profile. In some embodiments, measuring the metabolic profile comprises assaying the biological sample using liquid chromatography (LC), mass spectrometry (MS), liquid chromatography/mass spectrometry (LC/MS), or Ultrahigh Performance Liquid Chromatography-Tandem Mass Spectroscopy (UPLC-MS/MS). In some embodiments, the biological sample comprises CNS tissue or cerebrospinal fluid (CSF). In some embodiments, the CNS tissue is brain tissue. In some embodiments, the metabolic profile comprises a level of a first biomarker selected from the group consisting of glucose, glucose-6-phosphate, 3-phosphoglycerate, pyruvate, lactate, and phosphoenolpyruvate. In some embodiments, the metabolic profile comprises a level of a second biomarker selected from the group consisting of carnitine, malonylcarnitine, myristoylcarnitine, palmitoylcarnitine, malonylcarnitine, and beta-hydroxybutyrate.

In some embodiments, an rAAV particle comprising a nucleic acid molecule encoding ASPA or a pharmaceutical composition comprising the particle is administered to a subject systemically. In some embodiments, an rAAV particle comprising a nucleic acid molecule encoding ASPA or a pharmaceutical composition comprising the particle is administered to a subject intravenously; by direct injection via open surgery or laparoscopy; by injection into an artery or vein via catheterization. In any of the treatment methods described herein, the rAAV particle comprising a nucleic acid molecule encoding ASPA or the pharmaceutical composition comprising the particle disclosed herein may be administered to the subject using intravenous infusion.

The present disclosure further contemplates a use of a pharmaceutical agent (e.g., an rAAV or a pharmaceutical composition comprising an rAAV) described herein in the manufacture of a medicament for treating a disorder or a disease characterized by a malfunction or a deficiency of ASPA in a subject. The present disclosure also includes a use of a pharmaceutical agent (e.g., an rAAV or a pharmaceutical composition comprising an rAAV) described herein for treating a disorder or a disease characterized by a malfunction or a deficiency of ASPA in a subject.

The composition may be delivered in a volume of from about 50 microliters (μL) to about 1 mL, including all numbers within the range, depending on the size of the area to be treated, the viral titer used, the route of administration, and the desired effect of the method. In some embodiments, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 p L. In another embodiment, the volume is about 1000 μL.

In some embodiments, the lowest effective concentration of virus is utilized in order to reduce the risk of undesirable effects, such as toxicity or adverse immune response. Still other dosages in these ranges may be selected by the attending physician, taking into account the physical state of the subject (e.g., human) being treated, the age of the subject, the particular ASPA deficiency disorder, and the degree to which the disorder, if progressive, has developed.

In some embodiments, rAAV vectors or rAAV particles comprising a nucleic acid sequence encoding ASPA are administered to a subject at a dose ranging from about 10¹² to about 10¹⁶ vg/kg body weight of the subject, such as from about 10¹³ vg/kg to about 10¹⁵ vg/kg body weight of the subject. In some embodiments, rAAV vectors or rAAV particles comprising a nucleic acid sequence encoding ASPA are administered to a subject at a dose ranging from about 10¹⁴ vg/kg to about 5×10¹⁴ vg/kg. In some embodiments, rAAV vectors or rAAV particles comprising a nucleic acid sequence encoding ASPA are administered to a subject at a dose of about 1.32×10¹⁴ vg/kg. In some embodiments, rAAV vectors or rAAV particles comprising a nucleic acid sequence encoding ASPA are administered to a subject at a dose of about 3×10¹⁴ vg/kg.

In some embodiments, the therapeutically effective amount is in the range of about 10¹⁴ vg/kg to about 5×10¹⁴ vg/kg, for example, about 1.1×10¹⁴ vg/kg, about 1.2×10¹⁴ vg/kg, about 1.3×10¹⁴ vg/kg, about 1.4×10¹⁴ vg/kg, about 1.5×10¹⁴ vg/kg, about 2×10¹⁴ vg/kg, about 2.5×10¹⁴ vg/kg, about 3×10¹⁴ vg/kg, about 3.5×10¹⁴ vg/kg, about 4×10¹⁴ vg/kg, about 4.5×10¹⁴ vg/kg, or about 5×10¹⁴ vg/kg, including all values and subranges that lie therebetween. In some embodiments, the therapeutically effective amount is about 1.32×10¹⁴ vg/kg. In some embodiments, the therapeutically effective amount is about 3×10¹⁴ vg/kg. In some embodiments, the therapeutically effective amount is in the range of about 1.32×10¹⁴ vg/kg to about 3×10¹⁴ vg/kg

In some embodiments, the subject is administered a single dose of the rAAV vectors, rAAV particles, or compositions disclosed herein. In some embodiments, the subject is administered more than one dose of the rAAV vectors, rAAV particles, or compositions disclosed herein, for example, two, three, four, or five doses. In some embodiments, the subject is administered a single dose of the rAAV vectors, rAAV particles, or compositions disclosed herein via intravenous infusion.

The compositions disclosed herein may be administered to the subject at any frequency. In some embodiments, the composition may be administered to the subject once a day or more than once a day. In some embodiments, the composition may be administered to the subject twice, thrice, four, five, six, 7, 8, 9 or 10 times a day. In some embodiments, the composition may be administered to the subject every day, every alternate, every third day, every fourth day, every fifth day, or every sixth day. In some embodiments, the composition may be administered to the subject weekly, bi-weekly, or every three weeks. In some embodiments, the composition may be administered to the subject every 1, 2, 3, 4, 5, 6, 7, 8, 9, 10, or 11 months. In some embodiments, the composition may be administered to the subject every year. In some embodiments, the composition may be administered to the subject every 1, 2, 3, 4, 5, 10, 15, or 20 years. In a particular embodiment, the composition may be administered to the subject a single time during the subject's lifetime.

In some embodiments, any one of the methods disclosed herein further comprise administering a therapeutically effective amount of an immunosuppressant. In some embodiments, the methods comprise administering to the subject a therapeutically effective amount of an rAAV or a composition disclosed herein, and a therapeutically effective amount of an immunosuppressant. The disclosure provides methods of treating administering to the subject a therapeutically effective amount of an rAAV or a composition disclosed herein, and a therapeutically effective amount of an immunosuppressant.

In some embodiments, the immunosuppressant is administered before, concurrently with, and/or after the administration of the rAAV vector. In some embodiments, the immunosuppressant is administered before the administration of the rAAV vector. In some embodiments, the immunosuppressant is administered at least 12 hours before the administration of the rAAV vector. In some embodiments, the immunosuppressant is administered about 2 days before the administration of the rAAV vector.

In some embodiments, the immunosuppressant is a non-glucocorticoid immunosuppressant. In some embodiments, the immunosuppressant is an inhibitor of calcineurin. In some embodiments, the immunosuppressant is cyclosporin, tacrolimus, sirolimus, everolimus, zotarolimus, or any combination thereof. In some embodiments, the immunosuppressant is tacrolimus. Other non-limiting examples of immunosuppressants include alkylating agents such as nitrogen mustards (cyclophosphamide), nitrosoureas, platinum compounds, folic acid analogues, such as methotrexate, purine analogues, such as azathioprine and mercaptopurine, pyrimidine analogues, such as fluorouracil, protein synthesis inhibitors, cytotoxic antibodies such as dactinomycin, anthracyclines, mitomycin C, bleomycin, mithramycin, polyclonal antibodies inhibiting T lymphocytes, IL-2 receptor-directed monoclonal antibodies such as basiliximab or daclizumab, anti-CD3 monoclonal antibodies, such as muromonab, opioids, TNF-alpha binding proteins such as infliximab, etanercept, or adalimumab, mycophenolate, fingolimod and myriocin. Other examples of biological immune-suppressing agents include, but are not limited to, monoclonal antibodies, such as monoclonal antibodies that block the co-stimulatory pathway (e.g., appropriate antibodies against CTLA4, ICOS, CD80, OX40, or other targets), interfering RNA (e.g., siRNA, dsRNA, shRNA, miRNA, etc.) targeting immunostimulatory molecules (e.g., cytokines), and proteins (e.g., proteasome inhibitors).

In some embodiments, the immunosuppressant is administered orally. In some embodiments, the therapeutically effective amount of the immunosuppressant is in the range of about 0.005 milligrams per kilogram (mg/kg) to about 0.1 mg/kg, for example, about 0.01 mg/kg, about 0.015 mg/kg, about 0.02 mg/kg, about 0.025 mg/kg, about 0.03 mg/kg, about 0.035 mg/kg, about 0.04 mg/kg, about 0.045 mg/kg, about 0.05 mg/kg, about 0.06 mg/kg, about 0.07 mg/kg, about 0.08 mg/kg, about 0.09 mg/kg, or about 0.1 mg/kg, including all values and subranges that lie therebetween. In some embodiments, the therapeutically effective amount of the immunosuppressant is in the range of about 0.01 mg/kg to about 0.05 mg/kg. In some embodiments, the therapeutically effective amount of the immunosuppressant is about 0.025 mg/kg. In some embodiments, the therapeutically effective amount of the immunosuppressant is administered twice daily.

In some embodiments, the methods comprise administering a therapeutically effective amount of a steroid to the subject. In some embodiments, the steroid is a mineralocorticoid. In some embodiments, the steroid is a glucocorticoid. In some embodiments, the glucocorticoid is prednisolone, methylprednisolone, cortisol, cortisone, prednisone, dexamethasone, betamethasone, triamcinolone, beclomethasone, fludrocortisone, deoxy corticosterone (DOCA), aldosterone, or any combination thereof. In some embodiments, the steroid is hydrocortisone. In some embodiments, the steroid is administered before, concurrently with, and/or after administration of the rAAV vector. In some embodiments, the therapeutically effective amount of the steroid administered to the subject before the administration of the rAAV vector is higher than the therapeutically effective amount of the steroid administered to the subject after the administration of the rAAV.

In some embodiments, any one of the methods disclosed herein further comprise administering a therapeutically effective amount of an anti-histamine. In some embodiments, the anti-histamine is administered before, concurrently with, and/or after administration of the rAAV vector. In some embodiments, the anti-histamine is diphenhydramine, hydroxyzine, chlorpheniramine, or any combination thereof.

In some embodiments, the methods further comprise: (a) administering a small molecule metabolic modulator to the subject; (b) prescribing to the subject a dietary intervention, wherein the dietary intervention promotes glycolysis and/or reduces beta-oxidation in the subject; and/or (c) administering an immune-suppressing agent to the subject.

In some embodiments, after administering a vector as described herein, N-acetylaspartate (NAA) levels in urine, cerebrospinal fluid (CSF), and/or brain tissue are decreased. In some embodiments, after administration, NAA levels in urine are decreased. In some embodiments, NAA levels in urine are decreased at least about 10%, 15%, 20%, 30%, 40%, 50%, 60%, 70%, 80%, 90%, or greater. In some embodiments, NAA levels in urine remain reduced relative to pre-treatment levels for at least 6 months, 9 months, 12 months, 18 months, 24 months, or longer. In some embodiments, after administration, NAA levels in CSF are decreased. In some embodiments, NAA levels in CSF are decreased at least about 10%, 15%, 20%, 30%, 40%, 50%, 60%, 70%, 80%, 90%, or greater. In some embodiments, NAA levels in CSF remain reduced relative to pre-treatment levels for at least 6 months, 9 months, 12 months, 18 months, 24 months, or longer. In some embodiments, after administration, NAA, levels in brain tissue are decreased. In some embodiments, NAA levels in brain tissue are decreased at least about 10%, 15%, 20%, 30%, 40%, 50%, 60%, 70%, 80%, 90%, or greater. In some embodiments, NAA levels in brain tissue remain reduced relative to pre-treatment levels for at least 6 months, 9 months, 12 months, 18 months, 24 months, or longer.

In some embodiments, after administering a vector as described herein, new myelination is observable using magnetic resonance imaging (MRI).

### EXAMPLES

### Example 1: BBP-812 Gene Therapy Vector for Canavan Disease

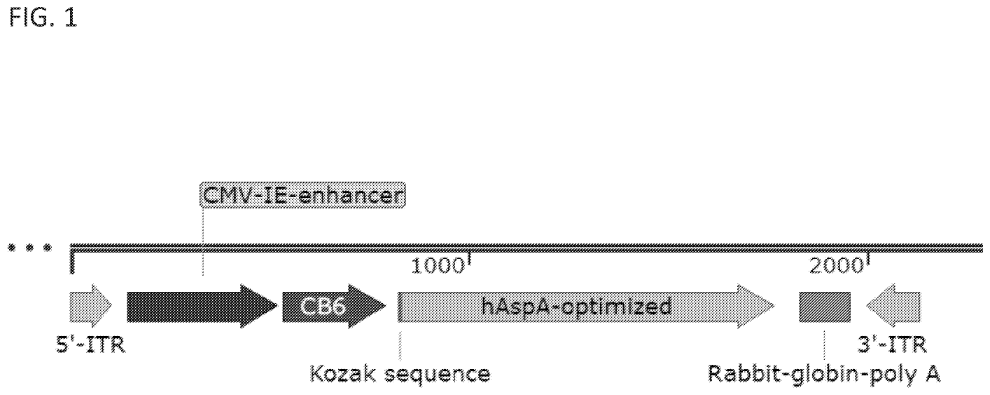

BBP-812 active substance is a non-replicating, rAAV serotype 9 (AAV9) vector containing a self-complementary expression cassette for the human ASPA transgene. The scAAV9-CB6-hASPAopt vector design is depicted in FIG. 1. It contains a codon-optimized human aspartoacylase transgene under the control of a CB6 promoter, including the cytomegalovirus immediate-early enhancer, flanked by 5′ and 3′ terminal repeats. The site of Rep nicking is removed in the 5′ inverted terminal repeat, thereby creating self-complementary AAV genomes.

BBP-812 is formulated as a sterile solution for injection in phosphate buffered saline, pH 7.0, 5% weight/volume (w/v) sorbitol, 0.001% w/v poloxamer 188. It is supplied in 5-milliliter (mL) pyrogen-free vials at a fill volume of 2.5 mL and sealed with a latex-free rubber stopper and aluminum flip-off seal. The vector concentration of the formulated product is between 1.8×10¹³ and 5.0×10¹³ vg/mL. The vials are stored frozen at <−60° C. until ready for use.

BBP-812 Drug Product (DP) is a single dose, preservative free, sterile solution, intravenous (IV) injection of non-replicating recombinant AAV9 vector at a concentration of 1.8×10¹³ to 4.2×10¹³ vg/mL. The BBP-812 DP solution contains 137 mM sodium chloride, 8.1 mM sodium phosphate dibasic, 1.47 mM potassium dihydrogen phosphate, 2.7 mM potassium chloride, 5% w/v sorbitol, 0.001% w/v poloxamer 188, and Water for Injection. BBP-812 DP is filled into 5 mL Crystal Zenith® (CZ, cyclic olefin polymer) vials with a nominal fill volume of 2.5 mL and stored at <60° C.

The study evaluates the safety, tolerability pharmacodynamic (PD) activity, and clinical activity of 2 dose levels of the AAV9 vector encoding ASPA (shown in FIG. 1) to pediatric patients of age less than or equal to 30 months with Canavan disease. Dosing is done up to and including the date before the participant turns 31 months old. Assessment of safety, tolerability, PD activity, and preliminary clinical activity of BBP-812 in the first 6 participants informs dose selection and enrollment expansion up to a total of 15 participants at the selected dose. All participants are followed for at least 5 years after the date of treatment with BBP-812.

BBP-812 is administered at a dose of 1.32×10¹⁴ vector genomes (vg) per kilogram (kg) body weight to Cohort 1, and at a dose of 3.0×10¹⁴ vg/kg body weight to Cohort 2. No placebo is administered in this study. Up to 18 participants are enrolled for the study, and a sample size of 15 participants are treated at a selected dose.

For pharmacodynamic analyses, changes in N-acetylaspartate (NAA) level after BBP-812 administration in urine and CNS tissue are measured. For clinical changes, motor, cognitive and language development and function will be evaluated after administration of BBP-812, for example, using brain imaging.

### Example 2: Nonclinical Studies of BBP-812

Demonstration of the therapeutic potential and safety of BBP-812 in treating Canavan disease is provided by nonclinical studies examining efficacy in an Aspa−/− mouse model of Canavan disease and safety evaluations in wildtype mice and in non-human primates. For the efficacy studies described below, heterozygous Aspa +/− mice were bred to generate homozygous pups. Animals were genotyped shortly after birth (PNDO/1) using a polymerase chain reaction (PCR) based assay utilizing genomic deoxyribonucleic acid (gDNA) template prepared from tail tissue samples. For efficacy studies, all mice were treated via IV injection into the facial vein on PND1. Nonclinical safety and pharmacology studies conducted with BBP-812 are summarized in Table 2. The main results of the studies are discussed in the following sections.

TABLE 2
Nonclinical Safety and Pharmacology Studies

| | | Species, Strain (Number, | | | | |
|---|---|---|---|---|---|---|
| Study Type/ | GLP | Sex and Age of Animals | Vector | | | |
| Study Number | Status | per Group) | Source | Duration | | Findings |
| Dose range | Non- | Aspa−/− mice | UMass | 365 | days | Dose-dependent restoration |
| finding study | GLP | N = 41 | vector | | | of normal brain NAA |
| | | PND1 | core | | | levels and motor function |
| Biodistribution | Non- | WT mice | UMass | 28 | days | Dose-dependent increases |
| | GLP | N = 120 | vector | | | in vector genome and |
| | | 6-8 weeks old | core | | | transgene mRNA in the |
| | | | | | | brain |
| Vector | Non- | WT mice | CMO | 28 | days | Similar transduction levels |

| | | | | | |
|---|---|---|---|---|---|
| comparability/ | GLP | N = 30 | Catalent | | as observed with UMass- |
| equivalence | | 6-8 weeks old | | | produced vector |
| study | | | | | |
| Biodistribution/ | Non- | NHPs | UMass | 21 and | Dose-dependent biodistribution |
| Safety | GLP | (cynomolgus monkeys) | vector | 56 days | to the brain and peripheral |
| | | N = 12 | core | | tissues, no observed safety |
| | | 2-2.5 years old | | | issues |

Abbreviations:
CMO: contract manufacturing organization;
GLP: Good Laboratory Practice;
mRNA: messenger ribonucleic acid;
NAA: N-acetylaspartic acid;
NHP: non-human primate;
PND: post-natal day;
UMass: University of Massachusetts;
WT: wildtype

### Canavan Disease Mouse Model

The Aspa−/− mouse model of Canavan disease was used to examine the safety and efficacy of BBP-812. The Aspa−/− mouse was the first animal model of Canavan disease developed and mimics the clinical phenotype of Canavan disease patients, presenting with ataxia, disequilibrium, muscle weakness, failure to thrive, craniofacial abnormalities, and cognitive impairment within the first month of life. Without intervention, Aspa−/− mice die around PND28 (Ahmed 2013).

While several other mouse models of Canavan disease have been reported, they present with a less severe phenotype (Carpinelli 2014, Mersmann 2011, Traka 2008). Notably, these other models all exhibit normal life spans and measurable motor function defects do not develop until several weeks of age. This phenotype is reminiscent of a milder form of Canavan disease (Janson 2006a, Mendes 2017, Yalcinkaya 2005).

For PoC studies, Aspa−/− mice were injected between PND 0 and 20 for initial experiments and at PND 1 for determination of the minimum effective dose (MED). From the literature, the neurological development in mice ranging from PND 0 to PND 20 is equivalent to the neuronal development of a human at 35 weeks gestation to ˜2.5 years of age (Dutta 2016). Therefore, the timing of treatment in the PoC studies supports the use of BBP-812 in pediatric patients aged <30 months old in a Phase 1/2 pediatric clinical trial (CVN 102).

### Dose-Rang Finding Efficacy Study in Post Natal Day 1 Aspa−/− Mice

A dose range finding study (Gessler 2017) was initiated to establish an MED in the animal disease model and to determine a starting dose for a proposed Phase 1/2 pediatric clinical trial CVN 102. Key parameters for determining the MED included survival and weight gain, reduction in CNS NAA levels, improvements in motor function, and improvements in brain histopathology. Biodistribution within the brain was also assessed.

### Study Design: Randomization and Dose Justification

Doses evaluated in the study were 2.6×10¹³, 8.8×10¹³, and 2.6×10¹⁴ vg/kg. The highest dose was based on previous studies with earlier generation vectors (Ahmed 2013). The mid and low doses represent a 3- and 10-fold reduction in vector. Mice received a single IV injection on PND 1 into the facial vein.

For these studies, animals were born between March 2014 and January 2016. Randomization was performed by dosing all animals in a litter similarly. Only 25% of the pups in a litter are Aspa−/−; therefore testing multiple doses within the same litter was not always feasible. Initial dosing included the highest dose, vehicle (0.9% NaCl) treatment, and untreated animals. As efficacy data were generated, lower doses were tested as additional litters were born to establish a minimally efficacious dose. However, throughout the duration of the study random litters received the highest dose or the vehicle or remained untreated to demonstrate consistency of results over time and across vector lots. A complete listing of animals used in the development of BBP-812 and establishment of a MED are listed in Table 3.

TABLE 3
Animals Used in Testing with BBP-812

| Treatment/ | Mouse | | # Animals | |
|---|---|---|---|---|
| Dose (vg/kg) | genotype | Vector | Treated | Duration |
| 2.6 × 10¹⁴ | Aspa−/− | BBP-812 | 32 | Up to one year |
| 8.8 × 10¹³ | Aspa−/− | BBP-812 | 42 | Up to one year |
| 2.6 × 10¹³ | Aspa−/− | BBP-812 | 8 | 28 days |
| 0.9% saline | Aspa−/− | BBP-812 | 12 | 28 days |
| Untreated | Aspa−/− | N/A | 25 | 28 days |
| 2.6 × 10¹⁴ | wild type | BBP-812 | 12 | Up to one year |
| Untreated | wild type | N/A | 61 | Up to one year |

### Study Design: Efficacy Parameters Measured in the Study

Survival and weight gain: Survival was monitored daily and weight was measured every other day from PND 1 to PND 32, then once every other week from PND 42 (6 weeks) to PND 364 (1 year).

Reduction in CNS and peripheral NAA levels: Reduction in CNS and peripheral NAA levels were measured by magnetic resonance spectrometry (MRS) and mass spectrometry.

Motor function and spatial memory testing: Mouse motor performance was assessed using accelerated rotarod for motor function and endurance, balance beam for vestibular function and ataxia, and inverted screen for grip strength. Except for the rotarod (Rotarod Series 8, IITC Life Science, Woodland Hills, CA), the equipment was built by the University of Massachusetts Medical School Machine Shop. For each motor function test, mice were injected and tested independently. Controls for these experiments included untreated Aspa−/− mice, Aspa−/− mice treated with 0.9% saline, and wildtype (WT) mice. Treatment received by the animals was blinded to the assessor and animals were tested over a >2-year period for all assessments in the study.

Accelerated rotarod: Mice were trained 2 days before the testing day for 3 runs each. On the testing day, mice were placed on the rotarod to acclimate for 1 minute. Each mouse was tested 3 times and the best value was used for analysis. The acceleration and timing were set to 4 to 40 rpm over 5 minutes.

Balance beam: Animals were placed in the middle of a horizontal wooden balance beam (1.5×100 cm) with pads underneath to protect mice that might fall off the beam. Latency to fall was recorded, with a 5-minute time limit for each trial. To increase the stringency of this test, we increased the cut off time from the previously used 3 minutes to 5 minutes. Animals were tested three times and the best value was counted.

Inverted screen: Mice were placed in the center of a grid (30 centimeters squared (cm²) with 25 millimeters squared (mm²) holes) in horizontal position and allowed to acclimate for 1 minute. The grid was turned slowly within 15 seconds to 125 degrees so that the mouse was hanging upside down. Time was measured until drop-off. The cutoff for PND 27 testing was 3 minutes as published previously. At all other time points, the cutoff was 5 minutes to increase the stringency of the test. Each animal was tested three times and the best value was counted.

Brain histopathology was assessed by hematoxylin and eosin (H&E) staining.

Biodistribution was determined by droplet digital polymerase chain reaction (ddPCR).

### Study Results: Survival and Weight Gain

BBP-812 treatment increased survival and promoted weight gain when compared to untreated Aspa−/− animals (FIG. 2). Untreated animals (red line) began losing weight around 2 weeks of age and died by 4 weeks of age. Treated animals gained weight comparable to WT animals and survived until the end of the experiment.

No unscheduled deaths were reported during the study that were attributed to treatment, as outlined in Table 4. Causes of death included malocclusion (n=2) and infanticide (n=5). Given the nature of these events, no further pathology or histopathology assessments were performed.

TABLE 4
Listing of Unscheduled Deaths

| ID | Gender | DOB | Group | Cause of Death | Day |
|---|---|---|---|---|---|
| 97a-8 | F | 23 Apr. 2014 | BBP-812 - | malocclusion | 56 |
| | | | high dose | | |
| 135b-4 | M | 16 Jul. 2014 | BBP-812 - | malocclusion | 70 |
| | | | high dose | | |
| 179a-2 | F | 7 Oct. 2014 | BBP-812 - | all killed by mom | 1 |
| | | | high dose | | |
| 179a-7 | F | 7 Oct. 2014 | BBP-812 - | all killed by mom | 1 |
| | | | high dose | | |
| 179b-6 | F | 7 Oct. 2014 | BBP-812 - | all killed by mom | 1 |
| | | | high dose | | |
| 179a-1 | F | 7 Oct. 2014 | UT | all killed by mom | 1 |
| 179a-5 | F | 7 Oct. 2014 | UT | all killed by mom | 1 |

Abbreviations:
DOB: date of birth;
F: female;
M: male;
UT: untreated

### Study Results: Reduction in CNS NAA Levels

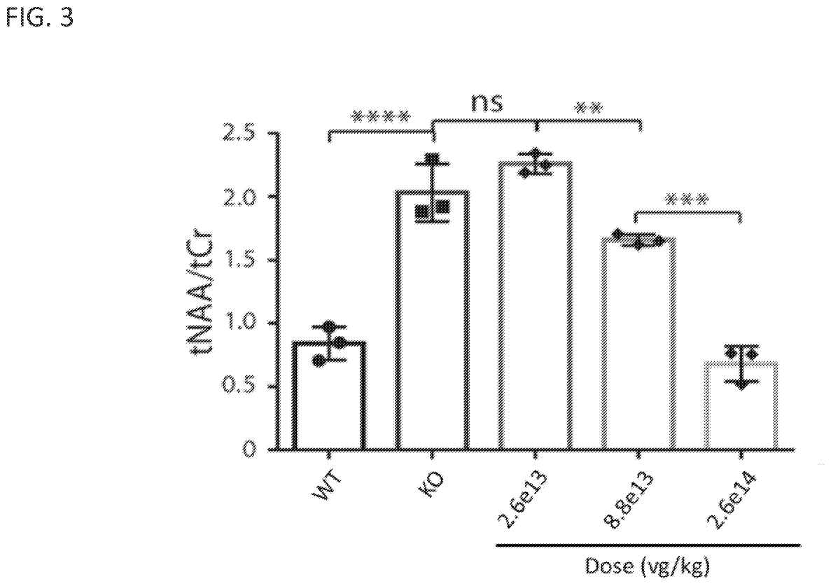

Using MRS analysis, animals receiving BBP-812 were monitored for CNS levels of NAA (FIG. 3 and Table 5). Animals receiving the 8.8×10¹³ vg/kg and 2.6×10¹⁴ vg/kg doses of BBP-812 had a decrease in CNS NAA and the 2.6×10¹⁴ vg/kg treatment group was statistically superior to the 8.8×10¹³ vg/kg group. No changes in NAA were observed with the lower 2.6×10¹³ vg/kg as compared to untreated Aspa−/− animals.

TABLE 5
Ratio of NAA/Creatine in the CNS of
Untreated and BBP-812-Treated Mice

| | Animal | BBP-812 Dose | | Group |
|---|---|---|---|---|
| Animal ID | Strain | (vg/kg) | tNAA/tCr | Average |
| 204b_1 | WT | — | 0.971 | 0.841 |
| 204a-10 | WT | — | 0.846 | |
| 208b-4 | WT | — | 0.706 | |
| 220a-3 | Aspa^{-/-} | — | 2.294 | 2.033 |
| 204a-6 | Aspa^{-/-} | — | 1.883 | |
| 211a_5 | Aspa^{-/-} | — | 1.922 | |
| 259N_R | Aspa^{-/-} | 2.6 × 10¹³ | 2.344 | 2.359 |
| 393b_5_O | Aspa^{-/-} | 2.6 × 10¹³ | 2.25 | |
| 441a_1_O | Aspa^{-/-} | 2.6 × 10¹³ | 2.484 | |
| 390a_4_O | Aspa^{-/-} | 8.8 × 10¹³ | 1.649 | 1.659 |
| 392a_4_O | Aspa^{-/-} | 8.8 × 10¹³ | 1.621 | |
| 392a_5_O | Aspa^{-/-} | 8.8 × 10¹³ | 1.706 | |
| 213b-7 | Aspa^{-/-} | 2.6 × 10¹⁴ | 0.764 | 0.680 |
| 228a_3 | Aspa^{-/-} | 2.6 × 10¹⁴ | 0.52 | |
| 227a_4 | Aspa^{-/-} | 2.6 × 10¹⁴ | 0.755 | |

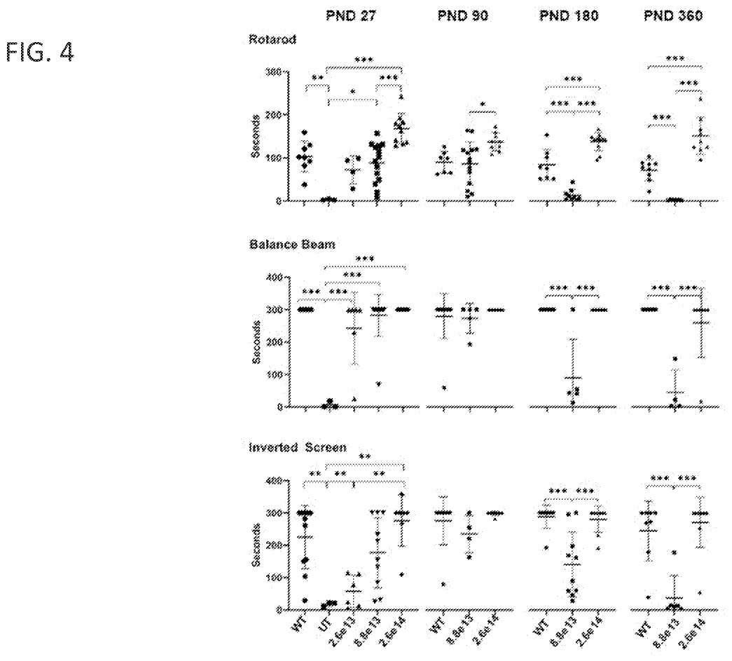

Abbreviations:
tCr: total creatine;
tNAA: total N-acetylaspartic acid;
vg/kg: vector genomes/kilogram;
WT: wild type
Individual data in the above table correspond to the data from FIG. 4.

### Function and Spatial Memory Testing

Mice were tested for improvements in three motor function assessments. At PND 27, the animals receiving 2.6×10¹³ vg/kg showed improvements as compared to untreated Aspa−/− mice but did not perform as well as WT animals. Motor function testing (rotarod, balance beam, and inverted screen) in mice treated with the 8.8×10¹³ vg/kg or 2.6×10¹⁴ vg/kg was comparable to WT mice at PND 27 and PND 90 in all 3 assessments. Results at PND 180 and PND 360 showed maintenance of the motor function rescue with 2.6×10¹⁴ vg/kg BBP-812.

Mice receiving 8.8×10¹³ vg/kg exhibited limited improvement in motor function at PND 180 and no benefit at the final time point (FIG. 4).

### Study Results: Brain Histopathology

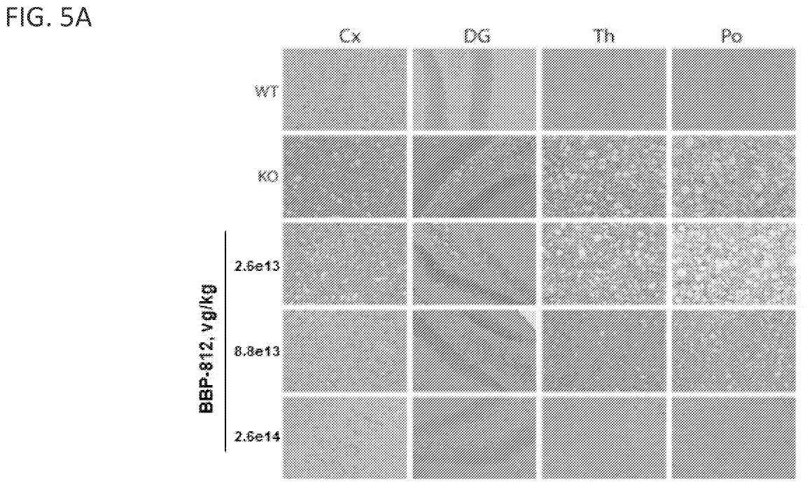

Brain histopathology in animals receiving BBP-812 was assessed by H&E staining and compared to WT and untreated Aspa−/− mice (FIG. 5A and FIG. 5B). The pathology from animals receiving the 2.6×10¹⁴ vg/kg dose was indistinguishable from WT animals. Animals receiving the 8.8×10¹³ vg/kg dose showed marked improvement with a reduction in vacuolation but some disease was still present (please note the pons (Po), cerebellum (Ce), and striatum (St) as examples). Mice treated with 2.6×10¹³ vg/kg showed little if any improvement when compared to untreated Aspa−/− mice.

### Study Results: Biodistribution

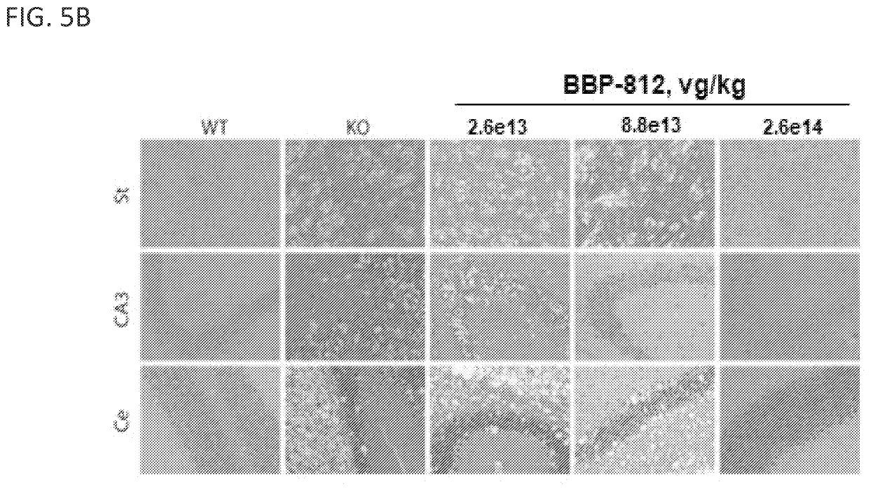

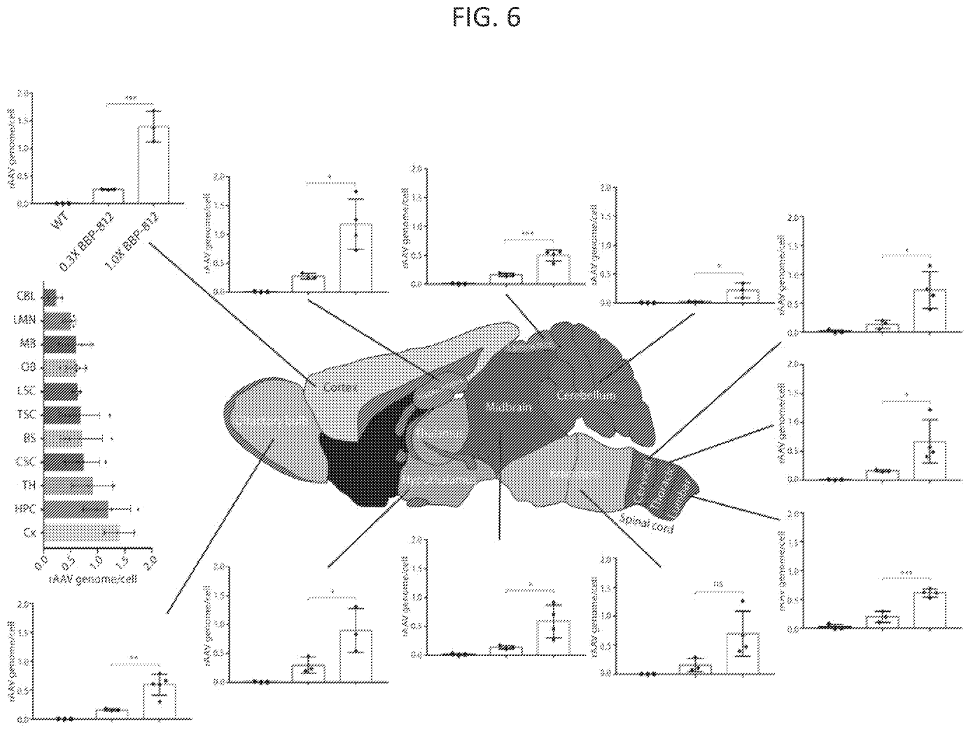

Vector genomes per diploid cell (vg/cell) were determined by ddPCR in samples from different regions of the brain (FIG. 6). Between 0.5 and 1.5 vg/cell were detected at 1-year post-dosing demonstrating that IV administration of BBP-812 can access and persist in all regions of the brain tested.

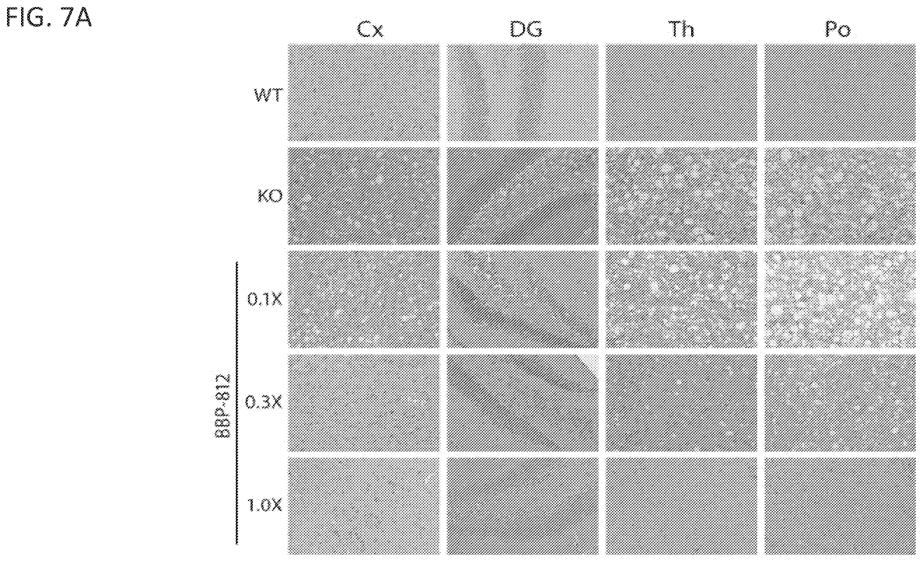

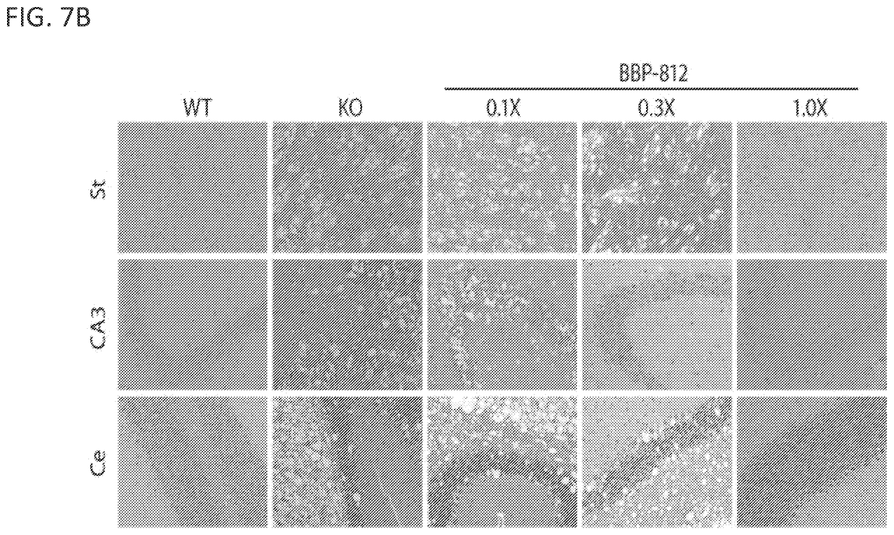

As a final determination of efficacy, brain histopathology in Aspa−/− mice receiving BBP-812 was assessed by H&E staining and compared to wildtype and untreated Aspa−/− mice (FIG. 7A and FIG. 7B). The histopathology from Aspa−/− animals receiving the 2.6×10¹⁴ vg/kg dose was indistinguishable from wildtype animals. Animals receiving the 8.8×10¹³ vg/kg dose showed marked improvement compared to untreated controls, with a reduction in vacuolization but some disease features still present (please note the pons [Po], cerebellum [Ce], and striatum [St] as examples). Aspa−/− mice treated with 2.6×10¹³ vg/kg showed little if any improvement when compared to untreated mice.

The results from these analyses demonstrated that a complete recovery of the disease phenotype could be achieved with doses of 2.6×10¹⁴ vg/kg and that a 10-fold lower dose provides a minimal benefit in motor function but not in NAA metabolism or histopathology.

The middle dose of 8.8×10¹³ vg/kg was sufficient to significantly improve all efficacy endpoints but not as well as the highest dose. Based on these findings, the MED of BBP-812 is approximately 8.8×10¹³ vg/kg.

### Safety and Biodistribution in Non-Human Primates

The purpose of the study was to evaluate safety (clinical observations, clinical chemistry [serum, urine, and CSF], hematology, immune response, and histopathology) and biodistribution of BBP-812 in the CNS and peripheral tissues. The study compared 3 routes of administration: IV into the saphenous vein, intrathecal (IT) into the lumbar subarachnoid space, and intracerebroventricular (ICV) into the left lateral ventricle. Animals were sacrificed at 3 and 8 weeks post dosing. The overall study design is outlined in Table 6.

TABLE 6
Design of 8-Week Safety and Biodistribution Study in Cynomolgus Macaques

| | | | | | Necropsy Interval | |
|---|---|---|---|---|---|---|
| | | | | | (Males/Females) | |
| | | | | Males/ | 3 Weeks | 8 Weeks |
| Group | Treatment | ROA | Dose^{1, 2, 3, 4} | Females | (Day 22) | (Day 57) |
| 1 | BBP-812 | IV | 3.14 × 10¹³ | 1/2 | 0/0 | 1/2 |
| | | | vg/kg | | | |
| 2 | BBP-812 | IV | 1.05 × 10¹⁴ | 1/2 | 0/0 | 1/2 |
| | | | vg/kg | | | |
| 3 | BBP-812 | IV | 3.14 × 10¹⁴ | 2/4 | 1/2 | 1/2 |
| | | | vg/kg | | | |
| 4 | Vehicle | ICV | 0 | 1/1 | 0/0 | 1/1 |
| 5 | BBP-812 | ICV | 3.59 × 10¹¹ | 1/2 | 0/0 | 1/2 |
| | | | total vg | | | |
| 6 | BBP-812 | ICV | 1.79 × 10¹² | 1/2 | 0/0 | 1/2 |
| | | | total vg | | | |
| 7 | BBP-812 | ICV | 8.98 × 10¹² | 2/4 | 1/2 | 1/2 |
| | | | total vg | | | |
| 8 | Vehicle | IT | 0 | 1/1 | 0/0 | 1/1 |
| 9 | BBP-812 | IT | 8.98 × 10¹² | 2/4 | 1/2 | 1/2 |
| | | | total vg | | | |
| 10 | BBP-812 | IT | 1.79 × 10¹² | 1/2 | 0/0 | 1/2 |
| | | | total vg | | | |
| 11 | AAV9- | IV | 1.69-1.75 × 10¹⁴ | 2/2 | 1/1 | 1/1 |
| | CB6- | | vg/kg | | | |
| | cynoASPA⁵ | | | | | |

Abbreviations:
ICV: intracerebroventricular;
IT: intrathecal;
IV: intravenous;
NHP: non-human primate;
ROA: route of administration
¹BBP-812 doses given as vg/kg for IV Groups 1 to 3 and 11
²BBP-812 doses given as total vg/brain for ICV Groups 5 to 7
³BBP-812 doses given as total vg/intrathecal space for IT Groups 9 and 10
⁴BBP-812 doses adjusted based on qualified ddPCR assay
⁵Control animals receiving NHP ASPA

### Study Design: Dose Justification

The doses were selected based on the minimum efficacious doses identified in PoC studies in the Aspa−/− mouse model of Canavan disease. The IV route of administration was compared directly to IT and ICV delivery to determine which route facilitated superior global CNS biodistribution in a large animal model.

### Study Design: Outcome Measures

Animals were routinely monitored throughout the study with daily clinical observations. Neurological evaluations were undertaken on Days 22 and 57. Blood was collected for clinical pathology and hematology at Days -1, 3, 8, 22, 36, and 57 post dosing. Antibody responses to AAV9 capsid and ASPA protein were determined prior to dosing and at Days 22 and 57 post dosing in the serum and CSF. Biodistribution was assessed as vector genomes per diploid cell as measured by droplet digital polymerase chain reaction (ddPCR) using a transgene specific TaqMan assay. Histopathology included H&E staining of all tissues and the CNS underwent additional immunostaining for ionized calcium-binding adaptor molecule 1 (IBA-1) and glial fibrillary acidic protein (GFAP). A minimum of eight dorsal root ganglia and associated spinal nerve roots were investigated per animal.

### Study Results: Biodistribution

Biodistribution of BBP-812 and expression of the hASPAOpt transgene were assessed in samples from animals in Groups 1, 2, 3, 7, and 9. Expression in the brain was assessed from 3 mm×3 mm tissue punches. A list of all punches collected and analyzed (bolded) from the brain can be seen in FIG. 8. A complete list of tissues analyzed, including spinal cord (thoracic, lumbar, and cervical) and peripheral organs, is shown in Table 7.

TABLE 7
Tissues Analyzed for Biodistribution of BBP-812

| Tissue | Day 22 | Day 57 |
|---|---|---|
| Deep frontal cortex | X | X |
| Caudate nucleus | X | X |
| Putamen | X | X |
| Thalamus | X | X |
| Hippocampus | X | X |
| Temporal cortex | X | X |
| Midbrain | X | X |
| Pons | X | X |
| Medulla | X | X |
| Visual cortex | X | X |
| Thoracic spinal cord | X | X |
| Lumbar spinal cord | X | X |
| Cervical spinal cord | X | X |
| Liver | X | X |
| Heart | X | X |
| Kidney | X | X |
| Lung | — | X |
| Lymph node | — | X |
| Spleen | — | X |
| Gonads | — | X |

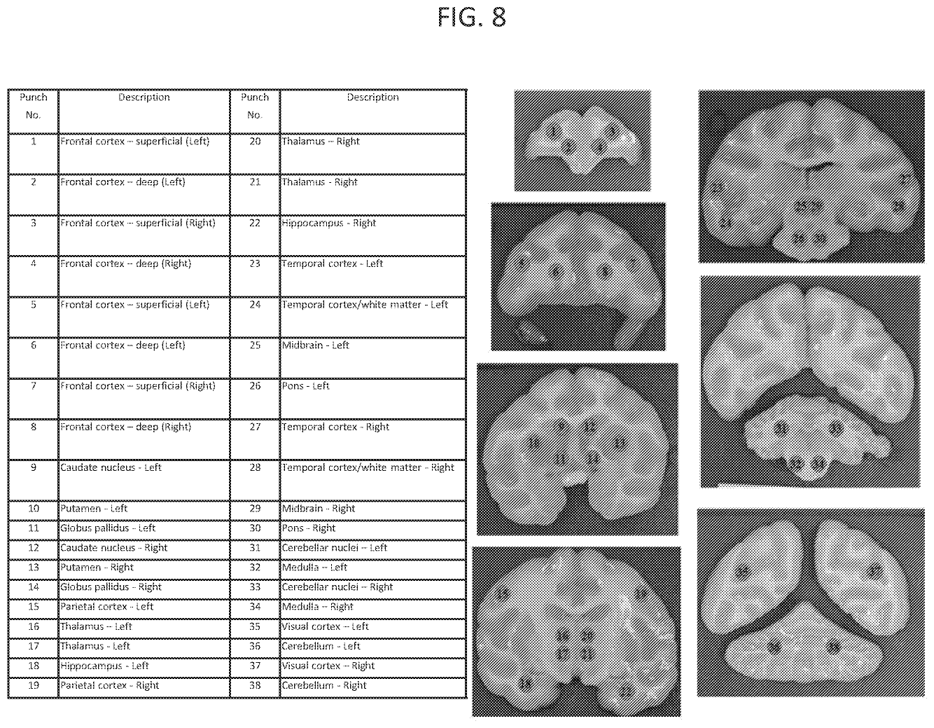

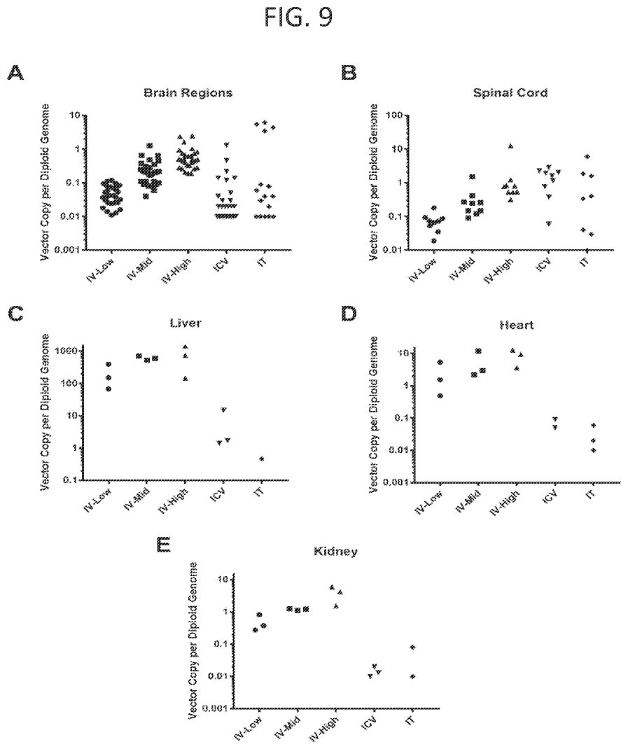

Intravenously treated animals presented with a dose-dependent increase in vector genomes across the CNS and in peripheral tissue. In the high dose group, vector genomes ranged from 0.26 to 2.16 in the brain punches at the Day 57 necropsy. Animals receiving IT or ICV administration of BBP-812 had significantly lower levels of transduction in the CNS as compared to all three IV doses. In the highest-dose IV animals, vector was detectable in all brain regions tested in every animal. In the IT and ICV dosing groups, vector was not detected in 5/30 and 13/30 brain punches respectively. Detection of vector genomes in peripheral tissues was also dose-dependent for the IV groups with the highest levels detected in the liver and heart. Animals receiving IT and ICV administration of BBP-812 had detectable genomes in peripheral tissues but at levels much lower than animals dosed by the IV route (FIGS. 9A-9E). Intravenous dosing was observed to penetrate far better than either IT or ICV into the deep cortical white matter and all other brain regions thought to be most relevant in Canavan disease.

### Study Results: Detection of Anti-AAV9 and Anti-ASPA Antibodies

All animals screened negative for neutralizing antibodies against AAV9 prior to randomization and dosing. Total antibodies against AAV9 and ASPA were measured before and after dosing in animals from groups 1, 2, 3, 7, and 9 using qualified enzyme-linked immunosorbent assays (ELISAs). Analysis of serum included samples collected prior to dosing and at days 22 and 57 post dosing (Table 8). No animals tested positive for antibodies against AAV9 prior to dosing. After dosing, 11/12 IV treated animals, 5/6 IT treated animals, and 5/6 ICV treated animals developed antibodies to AAV9. Prior to dosing, one animal in the high-dose IV group had detectable antibodies to ASPA. After dosing, 10/12 IV treated, 1/6 IT treated, and 1/6 ICV treated animal had antibodies to ASPA.

TABLE 8
Assessment of AAV9 and ASPA Antibodies in Serum Samples

| Treatment | Pre-Study Serum | | Day 22 Serum | | Day 57 Serum | |
|---|---|---|---|---|---|---|
| Group | AAV9 | ASPA | AAV9 | ASPA | AAV9 | ASPA |
| IV-low | 0/3 | 0/3 | 2/3 | 2/3 | 2/3 | 3/3 |
| IV-mid | 0/3 | 0/3 | 3/3 | 1/3 | 3/3 | 2/3 |
| IV-high | 0/6 | 1/6 | 6/6 | 5/6 | 3/3 | 2/3 |
| ICV | 0/6 | 0/6 | 5/6 | 0/6 | 3/3 | 1/3 |
| IT | 0/6 | 0/6 | 5/6 | 0/6 | 3/3 | 1/3 |

Abbreviations:
ICV: intracerebroventricular;
IT: intrathecal;
IV: intravenous

### Study Results: Neurological Examination

Animals underwent a neurological examination on Day 22 and Day 57. This included assessment of general sensory and motor function, cerebral reflexes, and spinal reflexes. There were no BBP-812-related changes found during these neurological examinations.

### Study Results: Clinical Chemistry and Hematology and Coagulation Assessment

There were no definitive BBP-812-related changes in hematology parameters, and coagulation values were unremarkable for all groups. Changes in serum chemistry were limited to transient elevation in liver enzymes in the high-dose IV group. These values returned to normal without intervention. There were no notable changes in urinalysis parameters for any of the animals. Changes in CSF chemistry after the administration of BBP-812 included decreased albumin on Day 22 and Day 57 for animals dosed via the IV route.

### Study Results: Necropsy and Histopathological Evaluation and Findings

A thorough histopathological examination was undertaken. At necropsy, the brain was cut in a brain matrix at 3 mm coronal slice thickness. The first slice and every other slice thereafter were fixed in 10% neutral buffered formalin for histological analysis. The spinal cord (cervical, thoracic, and lumbar) was cut into 1-cm sections, with the catheter tip defined as the zero point for the IT animals and the thoracolumbar junction for the IV animals. The first section and every second section thereafter (odd numbered) were fixed in 10% neutral buffered formalin for histopathological evaluation. A complete list of tissues analyzed and specific information regarding sampling and analysis are provided in Table 9.

TABLE 9
List of Tissues Collected for Histopathology During NHP Study
Tissue List

| | | | | | |
|---|---|---|---|---|---|
| Adrenals* | Femur (with | Lymph nodes | Seminal Vesicles | Sural nerve* | Urethra |
| | marrow) | (mesenteric)* | | | |
| Aorta | Gross Lesions* | Ovaries* | Skeletal muscle* | Testes (with | Urinary |
| | | | | epididymides)* | bladder |
| Brain* | Heart* | Pancreas | Skin (with mammary | Thymus | Uterus |
| | | | gland) | | |
| Cecum | Ileum | Pituitary | Spinal cord (cervical, | Thyroids (with | — |
| | | | thoracic, and lumbar)* | parathyroid) | |
| Colon | Jejunum | Prostate | Spinal nerve roots | Tibial nerve* | — |
| | | | and ganglion* | | |
| Duodenum | Kidneys* | Rectum | Spleen* | Trachea | — |
| Esophagus | Liver (with | Salivary glands | Sternum (bone and | Trigeminal | — |
| | gallbladder)* | | bone marrow) | ganglia* | |
| Eyes (with | Lungs (with | Sciatic nerve | Stomach | Ureter | — |
| optic nerves) | bronchi)* | (proximal and | | | |
| | | distal)* | | | |

*Tissue will be evaluated by microscopic histopathology; others stored as formalin-fixed paraffin-embedded

### Study Results: Histopatholozy Findings

There were no adverse test article-related microscopic or functional changes in NHPs following BBP-812 IV administration of doses up to 3.14×10¹⁴ vg/kg. Test article-related changes were observed only in the liver (portal infiltrates and/or increased cellularity [Kupffer cells]) of 2 of 3 animals dosed with 1.8×10¹⁴ vg/kg BBP-812 IV from the Day 22 and Day 57 necropsies. These changes are commonly observed in studies involving the IV administration of an AAV product and were not regarded as adverse.

Findings of cellular infiltrates in dorsal root ganglion (DRG) were reported in 3 of 6 BBP-812 treated IV high dose animals impacting a total of 4 out of 48 DRGs. Infiltrates were not associated with any evidence of neuronal degeneration and/or necrosis and were not distinguishable from the same findings routinely observed in control NHPs. The DRG findings for animals that received BBP-812 by IV administration were not adverse, had no impact on functional activity, and were of no toxicological consequence. A no observed adverse effect level (NOAEL) of 3.14×10¹⁴ vg/kg was determined from this study.

There were no adverse test article-related microscopic changes at IT doses up to and including 5.0×10¹² total vg BBP-812. There were 2 equivocal test article-related changes observed at the 5.0×10¹² total vg BBP-812 dose level. First, there was a mild increase of cellularity in a single dorsal root ganglion of 2 of 3 animals (Day 22 sacrifice). This was not associated with any neuronal changes and was not interpreted to be adverse. A similar change was not observed in animals necropsied on Day 57. Second, bilateral microgliosis (mild) of the ventral gray matter was present in 1 of 3 animals at the Day 22 sacrifice. This change was not associated with any apparent neuronal loss or degeneration, was not present in any other animal including the Day 57 animals, and may have been due strictly to enhanced staining of resident microglial cells. This level of microgliosis was not considered adverse.

### Nonclinical Toxicology: Study Design

A GLP-compliant safety and biodistribution study was conducted in wildtype C57BL/6 mice. The experimental design for this study is shown in Table 10. Three groups of C57BL/6 mice received a low (1×10¹⁴ vg/kg) or high (3×10¹⁴ vg/kg) dose of BBP-812 or vehicle via retro orbital injection on Day 0 and were monitored for 24 weeks. These BBP-812 dose levels are thought to be clinically relevant. Clinical observations, body weights, and food consumption were recorded for the duration of the study. Animals were euthanized at 4, 12, and 24 weeks after dosing. Tissue was collected, weighed, and preserved for histopathology, biodistribution analysis, and immunogenicity. Blood (terminal collection) was collected for hematology, clinical chemistry, biodistribution, and immunogenicity.

TABLE 10
Design of 24-Week Safety and Biodistribution Study in C57BL/6 Mice

| | | BBP-812 | Number per | | | |
|---|---|---|---|---|---|---|
| Group | | Dose | Time Point | | Endpoint | |
| Number | Description | (vg/kg) | (4, 12, and 24 wk) | Subsets | Tissue | Blood |
| 1 | Vehicle | NA | 5M/5F | 1-1 | Immunogenicity | Hematology |
| | | | 5M/5F | 1-2 | Biodistribution | Biodistribution |
| | | | | | | Immunogenicity |
| | | | 5M/5F | 1-3 | Histopathology | Clinical |
| | | | | | | Chemistry |
| 2 | Low | 1 × 10¹⁴ | 5M/5F | 2-1 | Immunogenicity | Hematology |
| | | | 5M/5F | 2-2 | Biodistribution | Biodistribution |
| | | | | | | Immunogenicity |
| | | | 5M/5F | 2-3 | Histopathology | Clinical |
| | | | | | | Chemistry |
| 3 | High | 3 × 10¹⁴ | 5M/5F | 3-1 | Immunogenicity | Hematology |
| | | | 5M/5F | 3-2 | Biodistribution | Biodistribution |
| | | | | | | Immunogenicity |
| | | | 5M/5F | 3-3 | Histopathology | Clinical |
| | | | | | | Chemistry |

Abbreviations:
F: female;
M: male;
vg/kg: vector genomes per kilogram;
NA: not applicable;
wk: week

### Dose Justification

The doses were selected based on the dose range finding study performed in the Aspa−/− mouse model of Canavan disease. The IV route of administration was selected because of the efficacy data generated in the Aspa−/− mouse and because of improved distribution to the CNS when compared to IT and ICV dosing observed in NHPs. The retroorbital route was performed to permit administration of the larger volumes needed to achieve the desired higher doses in mouse based on the concentration of vector used in the PoC studies. The doses administered in the GLP toxicology study support the proposed clinical dosing regimen. Age of the intervention is also a critical factor for the successful long-term outcome after correcting the underlying disease pathology.

### Outcome Measures Through Week 24

Animals underwent daily and then weekly clinical observations. Animals were sacrificed at Weeks 4, 12, and 24 post dosing whereupon peripheral blood samples were collected for clinical pathology and hematology. Immunoassays assessed antibodies against AAV9 and ASPA at each necropsy and T-cell reactivity was determined by enzyme-linked immune absorbent spot (ELISpot) against peptide pools for full-length AAV9 and human ASPA at all timepoints. Biodistribution was assessed by quantitative polymerase chain reaction (qPCR) for vector genomes and reverse transcription quantitative polymerase chain reaction (RT-qPCR) for transgene RNA. A comprehensive panel of tissues was collected for histological examination by H&E staining.

### Study Results: Clinical Observations Through Week 24

Detailed clinical observations were performed at randomization, on the day before dosing, and post dosing on Day 0, 1, 2, and 3, weekly for the first 12 weeks, and biweekly for the remainder of the study. Likewise, body weight was measured at randomization, on the day before dosing, and post dosing on Day 0, 1, 2, and 3, weekly for the first 12 weeks, and biweekly for the remainder of the study. Terminal body weights were collected at necropsy.

Finally, food consumption per cage and per week was monitored weekly for the first 12 weeks starting on the day of dosing, and then biweekly for the remainder of the study. No test article-related findings were noted.

### Study Results: Hematology and Clinical Chemistry Through Week 24

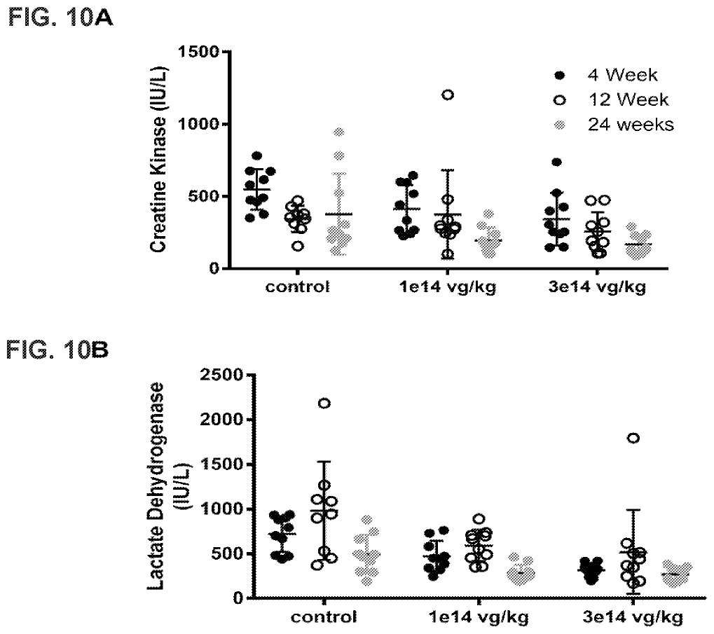

Hematology and clinical chemistry panels were assessed on all groups at 4, 12, and 24 weeks. There were no clinically meaningful test article related changes. There was a dose dependent decrease in creatine kinase and lactate dehydrogenase which was not associated with any microscopic changes (FIG. 10A and FIG. 10B).

Histopathology through Week 24

There was no test article related observations in the tissues examined.

Anti-Drug Antibody Responses through Week 24

Blood samples were collected for the analysis of circulating antibodies to AAV9 and human ASPA at the 4-, 12-, and 24 week necropsy. Blood was processed to serum and samples were stored at <−60° C. until analysis. Antibody titers were measured by a qualified ELISA.

At 4 weeks, no animals had antibody responses to transgene, and all animals that received BBP-812 developed antibodies to AAV9.

At 12 and 24 weeks, 1 vehicle treated animal at each timepoint screened positive for antibodies against AAV9 and only at 12 weeks, 1 animal receiving BBP-812 generated a mild antibody response to the transgene. All but 1 animal receiving BBP-812 developed antibodies to AAV9 (Week 12). An overview of the data is shown in Table 11 and is presented as number of positive animal/number of tested animals.

TABLE 11
Prevalence of Anti-AAV9 and Anti-ASPA Antibodies

| | 4 Week | | 12 Week | | 24 Week | |
|---|---|---|---|---|---|---|
| Treatment Group | AAV9 | ASPA | AAV9 | ASPA | AAV9 | ASPA |
| Group 1 (Vehicle) | 0/10 | 0/10 | 1/10 | 0/10 | 1/10 | 0/10 |
| Group 2 (BBP-812, 1 × 10¹⁴) | 10/10 | 0/10 | 9/10 | 0/10 | 10/10 | 0/10 |
| Group 3 (BBP-812, 3 × 10¹⁴) | 10/10 | 0/10 | 10/10 | 1/10 | 10/10 | 0/10 |

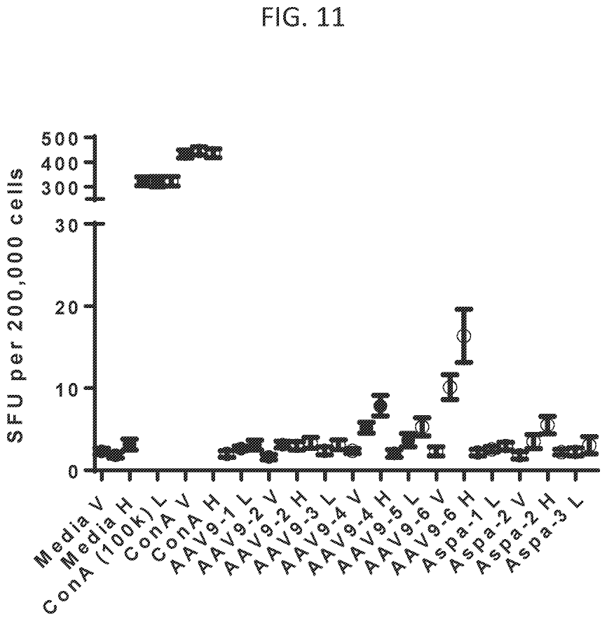

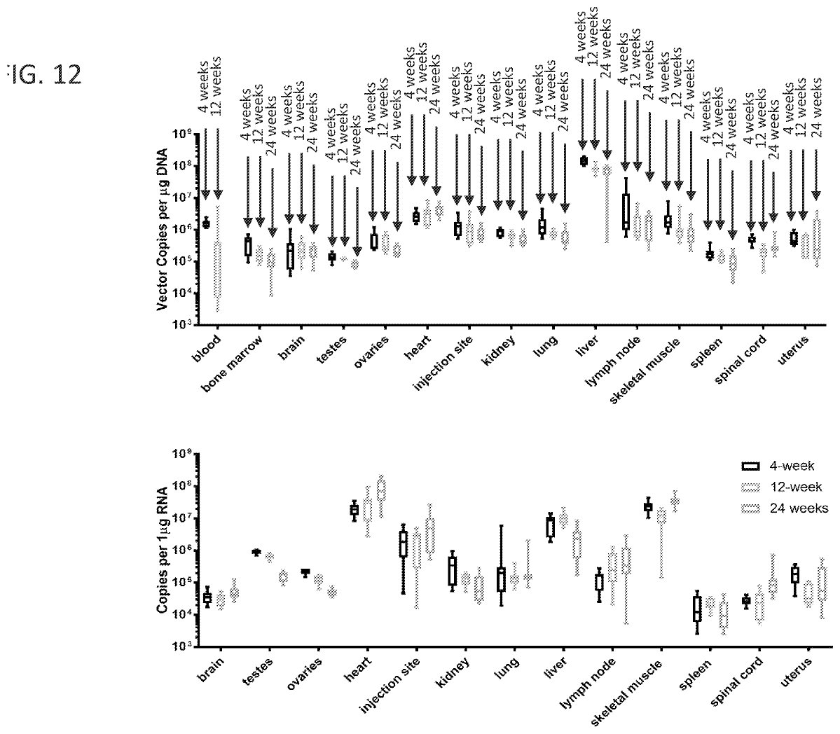

To assess cellular immune responses to AAV9 and ASPA, splenocytes were isolated from a cohort of animals from each dose group at the 12- and 24-week necropsies. Cells were stimulated with 15-mer peptide pools spanning the entire length of AAV9 and ASPA with a 2 amino acid overlap per peptide. Reactivity to the peptide pools was measured by interferon gamma secretion as determined by ELISpot assay. Concanavalin A (ConA) was used as a positive control. All tested samples showed a robust response to ConA, that was absent in media treated cells, demonstrating cellular health and responsiveness. There was no response to the ASPA peptides. Two of the AAV9 peptides pools showed a response in treated mice that was not observed in the control group. The observed values were slightly above the assay limit of detection and >20-fold lower than values observed with ConA (FIG. 11).

### Biodistribution Through Week 24

The tissues collected are listed in Table 12. Tissues were collected at each scheduled necropsy and flash-frozen in liquid nitrogen prior to storage at <−60° C. Blood was collected in K₂EDTA tubes and flash-frozen in liquid nitrogen prior to storage at <−60° C.

TABLE 12
Tissues Collected for Biodistribution Analysis
Organs/Tissues

Gross Lesions/Abnormal Tissue if sufficient tissue is available
Blood
Bone marrow
Brain
Gonads (Ovary/Testes)
Heart
Kidneys
Lungs
Liver
Lymph Nodes (Mesenteric)
Skeletal Muscle (Biceps femoris)
Spinal Cord
Spleen
Uterus

There was no detectable vector in tissues from vehicle-treated animals. All tissues from treated animals were positive for vector genomes and transgene RNA (FIG. 12). The highest levels of vector were detected in the liver. The highest levels of transgene RNA were in the heart, skeletal muscle, and liver.

Due to the lack of adverse findings in any of the parameters assessed, the NOAEL of BBP-812 under the conditions of this study was determined to be 3.0×10¹⁴ vg/kg.

### Example 3: A Phase 1/2 Open-Label Study of the Safety and Clinical Activity of Gene Therapy for Canavan Disease Through Administration of an Adeno-Associated Virus (AAV)

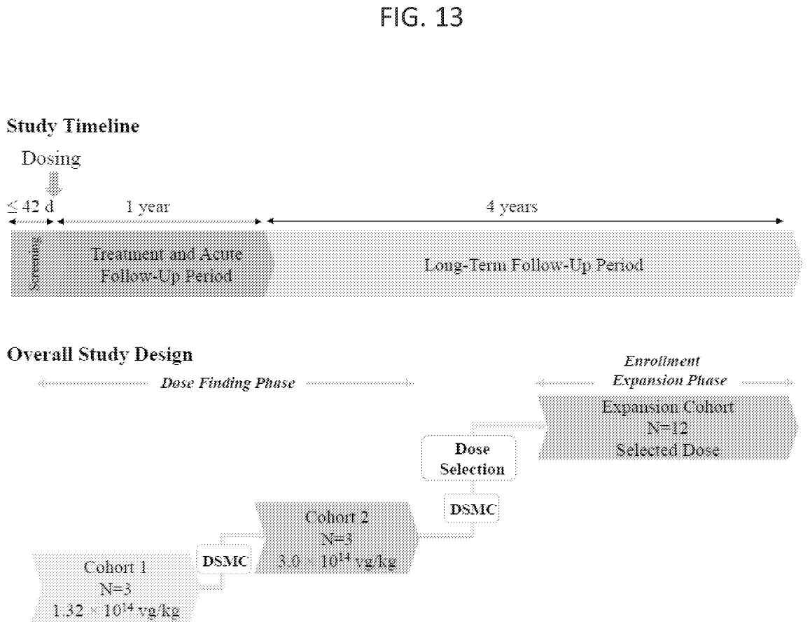

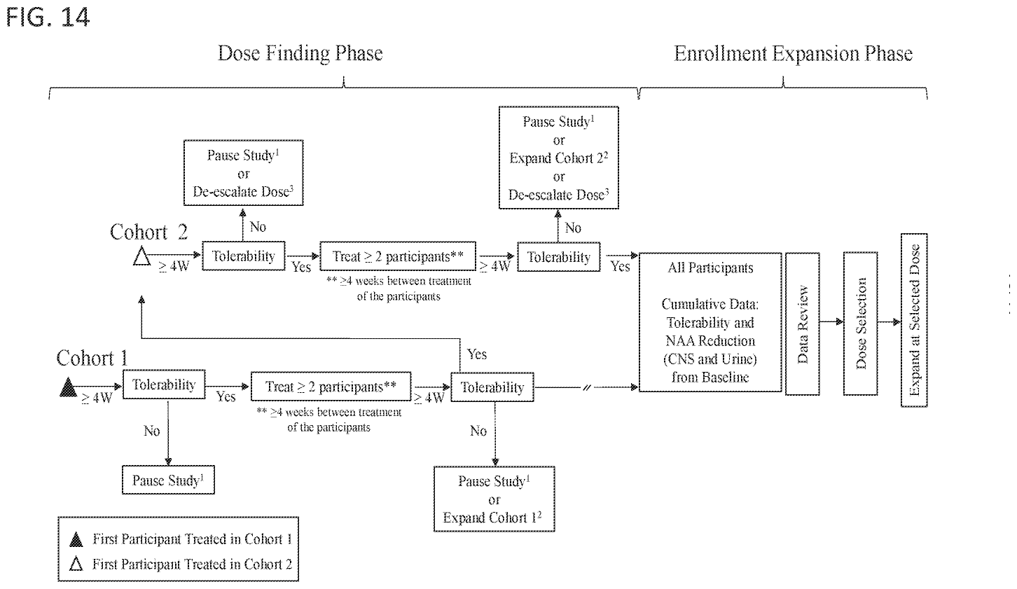

A Phase 1/2, open-label study of the safety and clinical activity of gene therapy for Canavan Disease through administration of an adeno-associated virus serotype 9 (AAV9)-based recombinant vector encoding the human ASPA gene (also referred to herein as “BBP-812”) is conducted. The study timeline and overall study design is depicted in FIG. 13.

The open-label, controlled study is designed to evaluate the safety, tolerability, pharmacodynamic (PD) activity, and clinical activity of 2 planned dose levels of BBP-812 administered to pediatric participants <30 months of age with Canavan disease. Assessment of safety, tolerability, PD activity, and preliminary clinical activity of BBP-812 in the first 6 participants will inform dose selection and enrollment expansion up to a total of 15 participants at the selected dose. All participants will be followed for at least 5 years after the date of treatment with BBP-812.

### Screening Period

After the patient's parent(s) and/or legal guardian(s) provide written consent, the patient becomes a study participant and is considered enrolled in the study. Study participants will be evaluated for treatment eligibility during the Screening Period. For all participants, the standard Screening Period will begin at the time of the first Screening assessment. The Screening Period will be up to 42 days before treatment with BBP-812 (Day 0). The Screening Period may be lengthened, if necessary, due to transient illness, unavoidable logistical challenges, or other factors determined by the Investigator. If >90 days elapse between key Screening assessments, those assessments will be repeated (i.e., re-screening).

### Baseline, Treatment, and Acute Follow-Up Periods

The total duration of the Baseline, Treatment, and Acute Follow-Up Periods is planned to be 52 weeks. A subsequent Long-Term Follow-Up Period will run for at least 4 years after the end of the Acute Follow-Up Period. In total, all participants will be followed for at least 5 years after the date of treatment with BBP-812.

After completion of the Screening procedures, treatment eligibility of the participant will be confirmed during the Baseline period. Participants who are eligible for treatment with BBP-812 will begin glucocorticoid prophylaxis on the day before BBP-812 dosing to prevent or dampen potential immune responses to BBP-812 and on the day of BBP-812 dosing will receive antihistamine prophylaxis to prevent infusion reactions. Participants will receive an intravenous (IV) infusion of BBP-812 on Day 0 conducted in an inpatient setting at a Sponsor-designated Treatment Center. Participants will receive only a single administration of BBP-812. After receiving BBP-812, participants will remain in the Sponsor-designated Treatment Center for safety observation for at least 72 hours after completion of dosing or longer as medically indicated per Investigator judgment. Upon hospital discharge, the participant will continue glucocorticoid prophylaxis for the first month after BBP-812 administration followed by a protocol-defined tapering regimen.

The study will use a conservative approach to selection of a viral vector dose in this pediatric/infant patient population with an ultra-rare disease. In total, at least 6 participants will be treated during the Dose Finding Phase, with at least 3 participants planned at each dose level. After the first participant receives BBP-812 at the starting dose, treatment of subsequent participants will occur only after a review of the first participant's safety data through at least the Day 28 post-dose visit. This review will be conducted by the Sponsor and Data and Safety Monitoring Committee (DSMC) to evaluate the safety and tolerability of BBP-812. In addition to ongoing Sponsor safety data reviews after each participant is dosed, DSMC and Sponsor reviews of at least 4 weeks of all participants' cumulative post-dosing safety data are required before dose escalation, dosing the second participant in a dose cohort, and before cohort expansion at a given dose level. Furthermore, before dose escalation proceeds to Cohort 2, the Month 3 MRI and CSF results from at least 1 participant in Cohort 1 will be evaluated. For Cohort 2, after at least 3 participants have been dosed and at least 4 weeks of safety data have been reviewed, dose expansion may be considered. Before proceeding with dose expansion, the Month 3 MRI and CSF results from at least 3 participants in Cohort 1 and from at least 1 participant in Cohort 2 will be evaluated. After 12-week post-dose data are available from the third participant treated in Cohort 2, all available data from the study participants, including safety and NAA levels, will be evaluated to inform dose selection and to support enrollment expansion up to a total of 15 participants at the selected dose level.

During the first 4 weeks post-treatment, all assessments will be conducted at the Treatment Center. After this time period, subsequent assessments are permitted at Follow-Up Centers and, depending on the type of assessment, at the participant's home.

Adverse events (AEs) and concomitant medication use will be monitored continuously. Standard safety assessments, laboratory measures, physical examinations, a standard 12 lead electrocardiogram (ECG), and a baseline electroencephalogram will be performed. Cerebrospinal fluid (CSF) samples will be obtained via lumbar puncture while the participant is under sedation/anesthesia at time points that coincide with MRI and MRS.

Immunogenicity will be assessed by evaluating the development of antibodies and T cell responses to capsid and transgene product. Biodistribution of AAV9 vector in blood and vector shedding in saliva, urine, and feces will be monitored. During the first 12 weeks after treatment with BBP-812, the participant's caregiver will be contacted weekly for assessment of safety status when such contact does not coincide with a scheduled study visit.

The PD activity of treatment with BBP-812 will be assessed by measuring NAA levels in urine and CNS. Brain NAA levels will be measured non-invasively by MRS and also directly in CSF via samples obtained by lumbar punctures that coincide with MRS time points.

Concurrently, the effect of treatment with BBP-812 on characteristics of brain anatomy and tissue composition will be assessed by MRI.

Functional measures will be assessed through scales and tests of fine and gross motor development and cognitive and language development. Adaptive behaviors of the participants and the quality of life (QOL) of the family/caregiver(s) will involve instruments that are validated in pediatric populations. The Canavan Disease Rating Scale will be used specifically for assessing neurological domains of participants with Canavan disease.

Standard neurological examinations will be conducted for evaluation of changes related to Canavan disease. In addition, ophthalmologic assessments including visual evoked potential (VEP) testing will be conducted.

### Long-Term Follow-Up Period

After the Treatment and Acute Follow-Up Periods, a Long-Term Follow-Up Period will run for at least 4 years. In addition to safety, assessments of motor development, cognitive and language development, QOL of the family of the family/caregiver(s), neurological status, and NAA levels will be followed to assess the durability of effects of treatment with BBP-812.

In total, all participants will be followed for at least 5 years after the date of treatment with BBP-812. A Long Term Follow Up Oversight Plan will describe a process of continual safety monitoring performed by the Sponsor in collaboration with the DSMC.

### Safety Monitoring

A DSMC will monitor and assess AEs, serious adverse events (SAEs), and post dose toxicities that occur during the study according to a study safety management plan and DSMC charter. The DSMC will review cumulative safety data to assess the safety and tolerability of BBP-812 in the context of continued treatment and enrollment in Cohort 1 and Cohort 2 during the Dose Finding Phase, dose selection, and enrollment and treatment during the Enrollment Expansion Phase of the study.

### Stopping Rules, Dose Limiting Toxicities, Dose Adjustment

This study may be paused, temporarily suspended, or prematurely terminated by the Sponsor.

Toxicity will be determined by the Common Terminology Criteria for Adverse Events (CTCAE) v5.0. Suspected dose limiting toxicities will include, but not be limited to, SAEs, Grade >3 AEs, or >Grade 3 laboratory AEs that are assessed by the Investigator as related to BBP-812 and are not otherwise attributable to Canavan disease or other causes. BBP-812 doses will not be adjusted on a per-participant basis.

### Prohibited Medications

Use of any gene therapy aside from BBP-812 is prohibited before and during the study. Use of other investigational medicinal products for the treatment of Canavan disease or other illnesses are not permitted within 30 days (or 5 half-lives, whichever is longer) before BBP-812 administration. Any hepatotoxic agents are prohibited for at least 4 weeks before and 12 weeks after receiving BBP-812. The participant's parent/caregiver should be advised to avoid giving the participant acetaminophen during the same period of time. In case of medical necessity, use of medications with hepatotoxic potential will be decided on an individual participant basis after discussion between the Investigator and the Sponsor. Investigators will make every effort to minimize hepatotoxic risk by selecting appropriate anti-epileptic medication as well as agents for sedation or anesthesia as required for protocol-specified assessments. Immunosuppressants may not be used at any time during the study, unless otherwise defined in the protocol.

### Number of Participants (Planned)

Planned Enrollment: Up to 18 dosed participants (total):
Dose Finding Phase: At least 6 participants
Enrollment Expansion Phase: Up to 12 participants Inclusion and Exclusion Criteria

### Inclusion Criteria:

A participant must meet all of the following criteria to be eligible for this study:
Is male or female <30 months of age as of the predicted date of BBP-812 infusion. (ie, dosing can occur up to and including the day before the participant turns 31 months old)
Has stable health in the opinion of the investigator and as confirmed by medical history and laboratory studies with no acute or chronic hematologic, renal, liver, immunologic, or neurologic disease (other than Canavan disease).
Has biochemical and genetic diagnosis of Canavan disease:
Elevated urinary NAA and Biallelic mutation of the ASPA gene determined at Screening or documented in the participant's medical history.
Has a clinical diagnosis and active signs of Canavan disease (including, but not limited to, hypotonia, developmental delay, and macrocephaly).
Is up to date on all immunizations according to geographically applicable guidelines (eg, Centers for Disease Control, World Health Organization) and the participant's parent(s) and/or legal guardian(s) consent to administration of standard as well as study-specific immunizations as required over the course of the study.
Has a parent(s) and/or legal guardian(s) who is willing and able to read/understand and provide written, signed informed consent after the nature of the study has been explained and before performance of study-related procedures.
Has a parent(s) and/or legal guardian(s) who is willing and able to permit participation of the participant in the study and to comply with all study requirements, including concomitant medication and other treatment restrictions.

### Exclusion Criteria:

A participant who meets any of the following criteria will be excluded from this study:
Tests positive for total anti-AAV9 antibodies (>1:50) as determined by enzyme-linked immunosorbent assay.
Received prior gene therapy or other therapy (including vaccines) involving AAV.
Is receiving high-dose therapy with immunosuppressants.
Has significantly progressed Canavan disease characterized as:
Presence of continuous/constant decerebrate or decorticate posturing,
Recurrent status epilepticus, or
Recalcitrant seizures that do not respond while on 3 or more anti-epileptic medications
Has an ASPA genotype known to be associated with a mild Canavan disease phenotype, per Investigator judgment.
Has any history of liver disease (eg, cirrhosis or current active liver dysfunction) as evidenced by medical history or clinical and/or laboratory findings during Screening.
Has abnormal laboratory values considered by the Investigator to be clinically significant:
Alanine aminotransferase or aspartate aminotransferase >upper limit of normal (ULN)
Conjugated bilirubin >ULN
Gamma-glutamyl transferase >ULN
Estimated glomerular filtration rate (eGFR) below the lower limit of normal for age
Hemoglobin <8 g/dL
White blood cell (WBC) count outside the normal range for age
Platelet count <150,000/pL
Partial thromboplastin time outside of the reference range
International normalized ratio outside of the reference range
Has evidence of active or latent infection by clinical and/or laboratory findings, including positive screening tests for severe acute respiratory syndrome coronavirus 2 (SARS CoV 2; i.e., coronavirus disease-2019 [COVID 19]), human immunodeficiency virus type 1 or 2 (HIV 1 or HIV 2), hepatitis B or C, or tuberculosis.
Has a confirmed diagnosis of hereditary fructose intolerance based on results of ALDOB genetic testing during Screening.
Is currently participating in another interventional clinical study or has completed another clinical study with an investigational drug or device within 30 days or 5 half-lives (per investigator discretion) before BBP-812 administration.
Has a past or current medical or behavioral condition, findings on physical examination or other medical assessments, or other circumstances that could, per Investigator judgment:
Adversely affect the safety and well-being of the participant during the study,
Interfere with completion of the study procedures or follow-up, or
Confound interpretation of the study results.
Has a history of hypersensitivity to any of the excipients of the study drug.

### Investigational Product and Placebo, Dosage, and Mode of Administration

Prophylaxis: On Day -1, at least 24 hours before dosing with BBP-812, participants will begin glucocorticoid prophylaxis with either prednisolone via feeding tube (if already present) or methylprednisolone via IV infusion to prevent or dampen potential immune responses associated with BBP-812 administration. The participant will continue glucocorticoid prophylaxis for the duration of the inpatient stay. On the day of BBP-812 dosing, participants will receive antihistamine prophylaxis to prevent infusion reactions. Upon hospital discharge, the participant will receive glucocorticoid prophylaxis with prednisolone (by feeding tube if already present, or orally) for the first month after BBP-812 administration.

An alternate glucocorticoid may be administered if medically indicated per Investigator judgment and after consulting with the Medical Monitor. At the Day 28 post-dose visit, the Investigator will determine whether to start glucocorticoid tapering according to protocol-defined guidelines.

Dosing: Treatment will be administered as follows: BBP-812 Cohort 1: 1.32×10¹⁴ vector genomes (vg) per kilogram (kg) body weight; BBP-812 Cohort 2: 3.0×10¹⁴ vg/kg body weight. As appropriate, a lower dose or interim dose of BBP-812 may be selected by the Sponsor based on its review of cumulative safety and activity data. Participants will be assigned sequentially to treatment Cohort 1 or 2 depending on the date of confirmation of treatment eligibility. BBP-812 will be administered at a Sponsor-designated Treatment Center as a 1 time, single IV infusion on Day 0 of the study. The duration of the infusion will depend on the dose level administered.

Dosing can occur up to and including the day before the participant turns 31 months old.

Placebo: No placebo will be administered in this study.

### Duration of Treatment and Study Participation

Each treatment-eligible participant will receive a single dose of BBP-812 administered by IV infusion on Day 0 of the study.

The maximum total duration of participation in the study will be at least 5 years.

Screening Period: up to 42 days (time may be lengthened, if necessary, due to transient participant illness, unavoidable logistical challenges, or other factors determined by the Investigator)

Treatment and Acute Follow-Up Period: 1 year

Long-Term Follow-Up: at least 4 years

### Statistical Methods

Analysis Populations: All participants who receive any amount of study drug will be included in the Safety Analysis Set. All participants who receive any amount of study drug and have at least 1 post baseline efficacy assessment will be included in the modified Intent to Treat (mITT) Analysis Set. Participants who discontinue will be included in the mITT Analysis Set, even if they do not have a post-baseline efficacy assessment. All participants in the mITT Analysis Set without any major protocol violations will be included in the Per-Protocol Analysis Set.

General presentations of data tabulations will be by age group. Summaries of continuous variables will include the total number (N), mean, median, standard deviation, minimum, maximum, and 2-sided 95% confidence intervals (CIs). Summaries of categorical data will include the counts and percentages, along with 2-sided 95% CIs. Kaplan-Meier methods will be used to summarize the time-to-event endpoints, including 25th, 50th (median), and 75th percentiles with associated 2-sided 95% CIs.

Analysis of efficacy parameters will be primarily based on all participants in the respective analysis populations; however, since efficacy may depend on the age of the participant at diagnosis, or depend on the time from diagnosis to treatment, additional exploratory analyses will be performed whereby participants will be stratified into appropriate categories, such as age groups and time from diagnosis (e.g., <12 months and >12 to 30 months of age; <12 months and >12 months from diagnosis). Comparisons to natural history data obtained from Study CVN-101 will be performed using matched patient data; specific details for matching methods, such as pair match, propensity scoring group matching, etc., will be specified in the statistical analysis plan.

Baseline for assessment of AEs related to BBP-812 or glucocorticoid dosing will be defined as just before start of administration; in general, designation of an AE as treatment emergent (i.e., treatment emergent adverse event [TEAE]) will begin on the date/time of treatment initiation. Baseline for analysis of change in all other variables/endpoints will be defined as the value closest, but before, drug product infusion; baseline will therefore be considered as either study Day -1, Day -2, Day -3, Day -4, Day -5, Day -6, Day -7, Day -8, Day −9, Day -10, or Screening for certain variables. Longitudinal data (collected serially over time on study and follow-up) will be presented by appropriate time intervals, such as monthly, quarterly, and so forth, depending on the nature of the data.

### Participant Disposition and Demographic Characteristics

Participant disposition will include the number of participants enrolled in each age group and the number and percentage of participants included in each analysis population by age group. The frequency and percentage of participants who prematurely discontinue from the study, along with the primary reason for discontinuation, will also be summarized.

Demographics and baseline characteristics, including age, sex, race, ethnicity, weight, length, and body mass index (BMI), as well as time from diagnosis and onset of clinical signs, will be summarized by age group.

### Safety Analyses

Statistical methods for the safety analyses will be primarily descriptive in nature and performed on the Safety Analysis Set. No imputation of missing safety data will be performed, other than of missing or partial dates, to be described in further detail in the statistical analysis plan. Participants will be categorized by age group.

The Medical Dictionary for Regulatory Activities version 23.1 (or later, as appropriate) will be used to code all AEs. Adverse events, including SAEs and TEAEs, will be summarized by System Organ Class and Preferred Term by age group and overall. TEAEs are defined as any AE occurring during or after administration of BBP-812. All TEAE summaries will include the number and percentage of participants experiencing an event, and the number of AEs experienced by the participants. Percentages will be based on the number of participants in the Safety Analysis Set within each age group. The potential for delayed safety effects of BBP-812 will be assessed as part of the planned long-term follow-up safety evaluation. AEs of severity Grade 3 or Grade 4, and those of at least 5% incidence, will be summarized. The relationship of TEAEs to BBP-812 and glucocorticoids will be summarized.

### Glucocorticoids

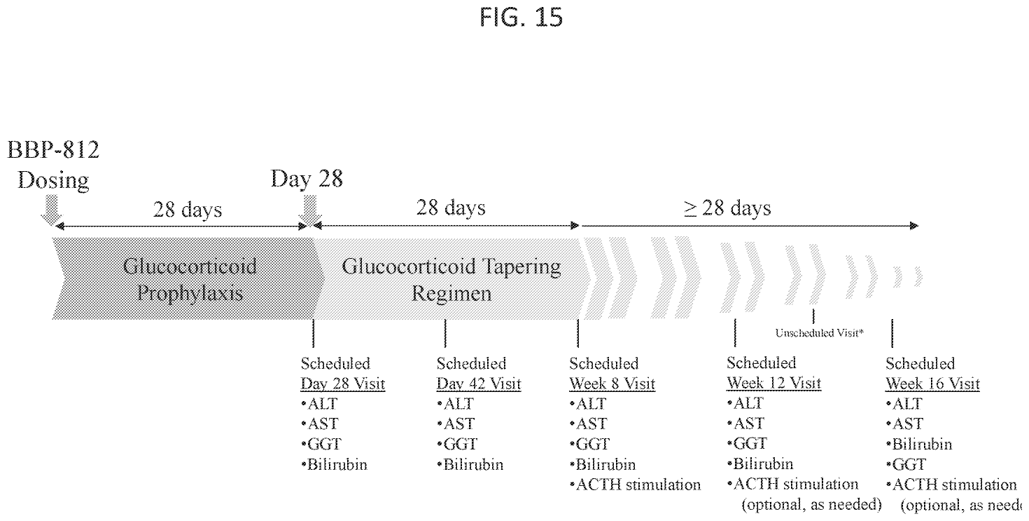

For all eligible participants, glucocorticoid prophylaxis will be administered to prevent or dampen potential immune responses associated with BBP-812 administration. The administration regimen is shows in FIG. 15. Antigen-specific T cell responses to AAV vector have been reported in pediatric patients 2 to 4 weeks after receiving gene therapy (Novartis 2021, Mendell 2017).

Prophylaxis: On Day -1, starting at least 24 hours before dosing with BBP-812 and continuing through the hospital stay, participants will receive glucocorticoid prophylaxis with prednisolone via feeding tube (if already present) or methylprednisolone via IV infusion. Upon hospital discharge, participants will receive glucocorticoid prophylaxis with oral prednisolone via feeding tube (if already present) or orally at least through the 28-day post treatment visit.

The Investigator may select a different glucocorticoid if clinically indicated. Safety laboratory testing and clinical assessment of the participant are used to guide whether glucocorticoid prophylaxis should be tapered, continued at the current dose, or increased (maximum 2.0 mg/kg/D prednisolone or dose equivalent). See below for the current recommended dosing and tapering regimen.

Participants will receive stress dose steroids for any intercurrent febrile illness or physiologic stress, using a regimen based on the Investigator's assessment of the individual participant's clinical status and local best practices. Adrenocorticotropic hormone (ACTH) stimulation testing will be performed to determine whether the hypothalamic-pituitary-adrenal (HPA) axis is intact and stress steroid use is no longer needed. It is not anticipated that the study glucocorticoid regimen will result in long-term adrenal insufficiency.

Based on review of safety data from the first dosed participant (see Example 4), the DSMC recommended moving to a higher starting dose of prednisolone as well as a more extended taper and increased monitoring, as outlined below.
2.0 mg/kg/d through Day 7
1.5 mg/kg/d through Day 29
1.0 mg/kg/d through Day 56 (Mo 2)
Begin taper and continue for at least 6 weeks with no more than a 15% decrease per week.
Monitor safety laboratories with liver function testing at least once weekly, or more frequently if any concerning trends are noted.
For events of AST/ALT elevation, obtain at least 1 creatinine kinase level to assess for potential non hepatic causes.

The regimen outlined above was used for the second dosed participant (see Example 4) and reflects the current base case. However, it is subject to additional modifications based on accrual of further clinical experience with BBP-812.

### Tapering

If there are no clinically significant laboratory or other findings consistent with liver inflammation 1 month after BBP-812 administration, then glucocorticoid prophylaxis may be tapered according to Investigator judgment and local clinical practice over a period of at least 28 days. Should liver abnormalities be noted on the Day 28 study visit results, in consultation with the Medical Monitor, daily glucocorticoid prophylaxis should be continued or increased to a maximum that is equivalent to a total daily dose of 2.0 mg/kg/day of oral prednisolone until the abnormalities resolve, then tapered as appropriate based on the dose level and duration of treatment according to Investigator and local clinical practice.

Safety laboratory testing and clinical assessment of the participant are recommended during the glucocorticoid taper.

ACTH stimulation testing will be performed to determine whether the HPA axis is intact. Until restoration of normal HPA axis functioning has been confirmed via the testing outlined above, participants will receive stress dose steroids for any intercurrent febrile illness or physiologic stress, using a regimen based on the Investigator's assessment of the individual participant's clinical status and local best practices. It is not anticipated that the protocol-mandated glucocorticoid dosing regimen will result in long-term adrenal insufficiency.

In addition, irrespective of glucocorticoid dose or duration, during the latter phase of the taper and prior to discontinuation of glucocorticoid prophylaxis, ACTH stimulation testing will be performed to determine whether the HPA axis is intact and whether glucocorticoid prophylaxis may be stopped completely. If HPA axis function has not fully recovered, patients will remain on their current glucocorticoid dose and a second ACTH stimulation test shall be performed at the next scheduled study visit (after approximately 4 weeks or more). ACTH stimulation testing is to be continued at study visits until HPA function has normalized, after which patients may discontinue the glucocorticoid and no longer require stress steroid coverage (see below). Specific methods, reference ranges, collection timing, and other details for testing should follow local practice standards and Investigator medical judgment. Until restoration of normal HPA axis functioning has been confirmed via the testing outlined above, participants will receive stress dose steroids for any intercurrent febrile illness or physiologic stress, using a regimen based on the Investigator's assessment of the individual participant's clinical status and local best practices. It is not anticipated that the protocol-mandated glucocorticoid dosing regimen will result in long-term adrenal insufficiency.

### Antihistamines

For all eligible participants, antihistamine prophylaxis will be used for the prevention of infusion reactions to BBP-812. Approved antihistamines include diphenhydramine (IV), hydroxyzine (intramuscular [IM]), and chlorpheniramine (IV), or the corresponding oral formulation administered via feeding tube (if already present). Selection of the antihistamine, dose, and route of administration will be based on the Investigator's medical judgment and local institutional practice.

### Other Preventative Treatment and Rescue Medications

In the event of anaphylactic reaction, according to the standard institutional procedures at the Treatment Center, the following therapies will be prepared for use: epinephrine, crystalloids, antihistaminic medications (e.g., diphenhydramine, hydroxyzine, or chlorpheniramine), and hydrocortisone. To address the potential development of CRS, antibodies against cytokines (e.g., tocilizumab, an IL-6R antibody) will be readily available for use. In addition, because episodes consistent with complement-mediated thrombotic microangiopathy have been reported with other systemic AAV9mediated gene therapy products (Pfizer 2019, Solid 2018, Solid 2019), some of which required anticomplement therapy, complement inhibitors (e.g., eculizumab, an antibody that blocks C5 cleavage, and a recombinant or plasma-derived C1 esterase inhibitor such as Berinert®) shall also be readily available. Please refer to the prescribing information for tocilizumab (Actemra® [Genentech 2019]), eculizumab (Soliris® [Alexion 2020]), and C1 esterase inhibitor (Berinert® [CSL Behring 2019]) for detailed information about these drugs.

Of note, eculizumab has been associated with serious meningococcal infections and, therefore, can only be ordered through a restricted program under a Risk Evaluation and Mitigation Strategy (Alexion 2020). Refer to the prescribing information for Soliris® (Alexion 2020) for information about meningococcal prophylaxis and the registration requirements to obtain eculizumab. Consultation with a medical specialist who is experienced with the use of eculizumab and management of complement activation syndromes/thrombotic microangiopathy is encouraged.

Clinically significant changes in physical examination findings, laboratory assessments, vital signs, and ECGs will be reported as AEs. Laboratory and vital sign data will be summarized by age group. All laboratory data and vital signs will be listed.

Concomitant medications data will be listed and summarized by age group. Study drug exposure will be summarized by age group.

### Pharmacodynamic Activity Analyses

There are several fundamental aspects of the analysis of NAA levels in urine and CNS (PD markers). Changes in NAA level after BBP-812 administration that are distinguishable from inherent within patient variability over time, in particular substantial decreases from Baseline, may be considered a marker of the physiological effect of the BBP-812 gene therapy, i.e., validation that NAA is a biomarker of effect. For these analyses, changes from Baseline may be evaluated using longitudinal data analysis methods. Furthermore, analyses of absolute values and changes from Baseline to post-treatment in clinical activity measures to assess correlations with decreases in NAA levels will be performed, as will analyses that assess potential differences in clinical outcome changes over time that may be associated with Baseline NAA level.

Statistical analysis of change in NAA levels from Baseline to specific timepoints will be performed using standard methods, such as a paired t-test; the primary timepoint analysis will be change to 12 months post-treatment with BBP-812. Changes from Baseline also may be evaluated using data analysis methods for longitudinal repeated measures. Furthermore, analyses of absolute values and changes in clinical activity measures from Baseline to post-treatment to assess correlations with decreases in NAA levels will be performed, as will analyses that assess potential differences in clinical outcome changes over time that may be associated with Baseline NAA level. Further details of PD and clinical activity assessments will be provided in the formal statistical analysis plan.

### Clinical Activity: Motor, Cognitive, and Language Development and Function; Adaptive Behavior

The mITT Analysis Set will be considered primary for the evaluation of clinical efficacy, and participants who discontinue the study without a post-baseline assessment of clinical efficacy will be considered as not responding to treatment. Several methods of imputation for analysis of such completely missing results may also be used, including assignment of the worst value across all treated participants (worst case analysis), and exclusion of such participants from analysis (best case analysis). For all clinical efficacy analyses, participants will be categorized by age group and duration from diagnosis.

Clinical analyses will be based on both absolute data and changes from Baseline over time in developmental tests. To detect potential efficacy signals and to evaluate the clinical importance of these absolute values and changes from Baseline, comparisons of clinical activity data to similar data collected in a natural history database of participants with Canavan disease in Study CVN-101 may be conducted.

### Other Assessments: Imaging Caregiver Questionnaires

The mITT Analysis Set will be considered primary for the evaluation of imaging and caregiver questionnaires. Participants who discontinue the study without post-baseline imaging assessments or questionnaire responses will be considered as not responding to treatment. For all imaging and questionnaire analyses, participants will be categorized by age group. Descriptive statistics will be used to present the data by age group.

Analyses will be related to brain MRI and brain MRS at Baseline and questionnaire responses at Baseline, and changes from Baseline.

### Sample Size

Enrollment for the study will be up to 18 dosed participants. The sample size of up to 15 participants treated at the selected dose would be appropriate for the assessment of the PD effect on NAA, based on similar but somewhat more conservative assumptions as used in a previously published protocol (Leone 2012), where a standard deviation of 0.493 mmol in the change from baseline was determined, and a difference from baseline of 2 to 3 mmol was considered to be important to detect. Using these assumptions for change from baseline in NAA, with a more conservative estimated standard deviation of 1.0 mmol and a clinically relevant difference of 2.0 mmol, the sample size of 15 would be more than adequate for greater than 90% power.

### Study Schedules

The schedule of events for screening and for the long-term follow-up period are shown in Table 13 and Table 14, respectively.

TABLE 13
Study Schedule of Events: Screening, Baseline, and Treatment and Acute Follow-Up Periods

| | | | Baseline, Treatment, and Acute Follow-Up Periods | | | | | | | | | |
|---|---|---|---|---|---|---|---|---|---|---|---|---|
| | | | Study Day | | | | | | | | | |
| | | | Baseline^{3, 1} | | | | | | | | | |
| | | | Day −10 | | | | | | | | | |
| | | | to | | | | | | | | | |
| Study | | | Predose | Dosing | Inpatient | | | Out-Patient | | | | |
| Assessment^{1, 2} | SCR³ | Re-SCR¹ | Day 0 | Day 0 | 1 | 2 | 3 | 4 | 5 | 6 | 7 | 10 ± 1 |
| Informed | X | X | — | — | — | — | — | — | — | — | — | — |
| consent | | | | | | | | | | | | |
| Demographics⁴ | X | — | — | — | — | — | — | — | — | — | — | — |
| Medical/Surgical | X | X | — | — | — | — | — | — | — | — | — | — |
| history⁵ | | | | | | | | | | | | |
| Confirm | — | — | X⁶ | — | — | — | — | — | — | — | — | — |
| eligibility | | | | | | | | | | | | |
| Admission to | — | — | X | — | — | — | — | — | — | — | — | — |
| inpatient unit | | | | | | | | | | | | |
| Physical | X⁷ | X⁷ | X⁸ | X⁸ | X⁸ | X⁸ | X⁷ | X⁸ | X⁸ | X⁸ | X⁷ | X⁸ |
| examination | | | | | | | | | | | | |
| (full or | | | | | | | | | | | | |
| directed) | | | | | | | | | | | | |
| Neurological | X | X | X | — | — | — | X | — | — | — | X | — |
| examination⁹ | | | | | | | | | | | | |
| Ophthalmologic | — | — | X | — | — | — | — | — | — | — | — | — |
| examination¹⁰ | | | | | | | | | | | | |
| Vital signs | X¹¹ | X¹¹ | X¹¹ | X | X¹¹ | X¹¹ | X¹¹ | X¹¹ | X¹¹ | X¹¹ | X¹¹ | X¹¹ |
| and pulse | | | | | | | | | | | | |
| oximetry¹¹ | | | | | | | | | | | | |
| Growth | X | X | X | — | X | X | X | — | — | — | X | — |
| assessments¹² | | | | | | | | | | | | |
| 12-lead ECG | — | — | X | — | — | — | — | — | — | — | X | — |
| Electroencep | — | — | X | — | — | — | — | — | — | — | — | — |
| halogram | | | | | | | | | | | | |
| (EEG)¹³ | | | | | | | | | | | | |
| Laboratory | — | — | — | — | — | — | — | — | — | — | — | — |
| assessments¹⁴ | | | | | | | | | | | | |
| Routine | X | X | X | — | X | X | X | — | X | — | X | — |
| clinical | | | | | | | | | | | | |
| laboratory | | | | | | | | | | | | |
| tests¹⁵ | | | | | | | | | | | | |
| Coagulation¹⁶ | X | X | X | — | — | — | — | — | — | — | — | — |
| Urine | X | — | X | — | — | — | — | — | — | — | — | — |
| NAA | | | | | | | | | | | | |
| Genetic | X | — | — | — | — | — | — | — | — | — | — | — |
| testing¹⁷ | | | | | | | | | | | | |
| Biomarkers | X¹⁸ | — | — | — | — | — | — | — | — | — | — | — |
| Archived | — | — | X¹⁹ | — | — | — | — | — | — | — | — | — |
| serum | | | | | | | | | | | | |
| sample | | | | | | | | | | | | |
| Immunogenicity: | X | — | — | — | — | — | — | — | — | — | — | — |
| Neutralizing | | | | | | | | | | | | |
| antibodies²⁰ | | | | | | | | | | | | |
| Immunogenicity: | X | X | — | — | — | — | — | — | — | — | X | — |
| Total | | | | | | | | | | | | |
| antibodies²¹ | | | | | | | | | | | | |
| Immunogenicity: | X | — | — | — | — | — | — | — | — | — | — | — |
| T-cell | | | | | | | | | | | | |
| reactivity²² | | | | | | | | | | | | |
| Pathogen | X | X | — | — | — | — | — | — | — | — | — | — |
| screening²³ | | | | | | | | | | | | |
| Blood | — | — | — | X²⁴ | — | — | — | — | — | — | — | — |
| biodistribution²⁴ | | | | | | | | | | | | |
| Vector | — | — | — | X²⁴ | X | X | X | | | | X | X |
| shedding²⁴ | | | | | | | | | | | | |
| Cerebrospinal | — | — | X²⁷ | — | — | — | — | — | — | — | — | — |
| fluid²⁶ | | | | | | | | | | | | |
| Imaging: | — | — | X²⁷ | — | — | — | — | — | — | — | — | — |
| brain MRI | | | | | | | | | | | | |
| Imaging: | — | — | X²⁷ | — | — | — | — | — | — | — | — | — |
| brain MRS | | | | | | | | | | | | |
| Administered | X | — | X | — | — | — | — | — | — | — | — | — |
| developmental | | | | | | | | | | | | |
| tests | | | | | | | | | | | | |
| (remote and | | | | | | | | | | | | |
| in-person)²⁸ | | | | | | | | | | | | |
| Questionnaires: | X | — | X | — | — | — | — | — | — | — | — | — |
| Caregiver | | | | | | | | | | | | |
| as | | | | | | | | | | | | |
| Respondent²⁹ | | | | | | | | | | | | |
| Immune/Infusion | — | — | X³⁰ | X^{30, 32} | X³⁰ <--------------------------------------------------------------------------------------> X | | | | | | | |
| prophylaxis | | | | | | | | | | | | |
| Steroid | — | — | — | — | — | — | — | — | — | — | — | — |
| tapering / | | | | | | | | | | | | |
| HPA axis | | | | | | | | | | | | |
| monitoring | | | | | | | | | | | | |
| BBP-812 | — | — | — | X^{11, 33} | — | — | — | — | — | — | — | — |
| administration³³ | | | | | | | | | | | | |
| Discharge | — | — | — | — | — | — | X³⁴ | — | — | — | — | — |
| from | | | | | | | | | | | | |
| inpatient unit | | | | | | | | | | | | |
| Caregiver | — | — | — | — | — | — | — | X³⁵ <-------------------------------------------------- ------------------------------------------> X | | | | |
| contact | | | | | | | | | | | | |
| (safety | | | | | | | | | | | | |
| status) | | | | | | | | | | | | |

| | | | |
|---|---|---|---|
| Survival | — | — | X <-------------------------------------------------------------------------------- |
| status | | | -------------------------------------------------------------------------------- |
| | | | ----------------------------------------------------------> X |
| Adverse | X³⁶ | — | X <-------------------------------------------------------------------------------- |
| events | | | -------------------------------------------------------------------------------- |
| | | | ----------------------------------------------------------> X |
| Concomitant | X | — | X <-------------------------------------------------------------------------------- |
| medications | | | -------------------------------------------------------------------------------- |
| | | | ----------------------------------------------------------> X |

| | Baseline, Treatment, and Acute Follow-Up Periods | | | | | | | | | | | |
|---|---|---|---|---|---|---|---|---|---|---|---|---|
| | | | | | Study Week (W) (±3 d) | | | | | | | |
| | Study Day | | | | Study Month (M) | | | | | | | |
| Study | Out-Patient | | | | W 8 | W 12 | W 16 | W 26 | W 34 | W 42 | W 52 | |
| Assessment^{1, 2} | 14 ± 2 | 21 ± 0 | 28 ± 2 | 42 ± 2 | M 2 | M 3 | M 4 | M 6 | M 8 | M 10 | M 12 | ET ± 3 d |
| Informed | — | — | — | — | — | — | — | — | — | — | — | — |
| consent | | | | | | | | | | | | |
| Demographics⁴ | — | — | — | — | — | — | — | — | — | — | — | — |
| Medical/Surgical | — | — | — | — | — | — | — | — | — | — | — | — |
| history⁵ | | | | | | | | | | | | |
| Confirm | — | — | — | — | — | — | — | — | — | — | — | — |
| eligibility | | | | | | | | | | | | |
| Admission to | — | — | — | — | — | — | — | — | — | — | — | — |
| inpatient unit | | | | | | | | | | | | |
| Physical | X⁷ | X⁷ | X⁷ | X⁷ | X⁷ | X⁷ | X⁷ | X⁷ | X⁷ | X⁷ | X⁷ | X⁷ |
| examination | | | | | | | | | | | | |
| (full or | | | | | | | | | | | | |
| directed) | | | | | | | | | | | | |
| Neurological | X | X | X | X | X | X | X | X | X | X | X | X |
| examination⁹ | | | | | | | | | | | | |
| Ophthalmologic | — | — | — | — | — | — | — | X | — | — | X | X |
| examination¹⁰ | | | | | | | | | | | | |
| Vital signs | X¹¹ | X | X | X | X | X | X | X | X | X | X | X |
| and pulse | | | | | | | | | | | | |
| oximetry¹¹ | | | | | | | | | | | | |
| Growth | X | — | X | | X | X | X | X | X | X | X | X |
| assessments¹² | | | | | | | | | | | | |
| 12-lead ECG | — | — | X | — | — | X | — | X | — | — | X | X |
| Electroencep | — | — | — | — | — | — | — | — | — | — | — | — |
| halogram | | | | | | | | | | | | |
| (EEG)¹³ | | | | | | | | | | | | |
| Laboratory | — | — | — | — | — | — | — | — | — | — | — | — |
| assessments¹⁴ | | | | | | | | | | | | |
| Routine | X | X | X | X | X | X | X | X | X | X | X | X |
| clinical | | | | | | | | | | | | |
| laboratory | | | | | | | | | | | | |
| tests¹⁵ | | | | | | | | | | | | |
| Coagulation¹⁶ | — | — | X | | X | | X | X | X | X | X | X |
| Urine | — | — | X | | X | X | X | X | X | | | X |
| NAA | | | | | | | | | | | | |
| Genetic | — | — | — | — | — | — | — | — | — | — | — | — |
| testing¹⁷ | | | | | | | | | | | | |
| Biomarkers | — | — | — | — | — | — | — | X | X | X | X | X |
| Archived | — | — | — | — | — | — | — | — | — | — | — | — |
| serum | | | | | | | | | | | | |
| sample | | | | | | | | | | | | |
| Immunogenicity: | — | — | X | — | — | X | — | X | — | — | X | X |
| Neutralizing | | | | | | | | | | | | |
| antibodies²⁰ | | | | | | | | | | | | |
| Immunogenicity: | X | X | X | — | X | X | — | X | — | — | X | X |
| Total | | | | | | | | | | | | |
| antibodies²¹ | | | | | | | | | | | | |
| Immunogenicity: | — | — | — | X | — | X | — | X | — | — | X | X |
| T-cell | | | | | | | | | | | | |
| reactivity²² | | | | | | | | | | | | |
| Pathogen | — | — | — | — | — | — | — | — | — | — | — | — |
| screening²³ | | | | | | | | | | | | |
| Blood | — | — | — | X | — | — | — | X | — | — | X | X |
| biodistribution²⁴ | | | | | | | | | | | | |
| Vector | X | X | X | | X | X | X | X | X | X | X | X²⁵ |
| shedding²⁴ | | | | | | | | | | | | |
| Cerebrospinal | — | — | — | — | — | X | — | X | — | — | X | X |
| fluid²⁶ | | | | | | | | | | | | |
| Imaging: | — | — | — | — | — | X | — | X | — | — | X | X |
| brain MRI | | | | | | | | | | | | |
| Imaging: | — | — | — | — | — | X | — | X | — | — | X | X |
| brain MRS | | | | | | | | | | | | |
| Administered | — | — | — | — | X | — | X | X | X | X | X | X |
| developmental | | | | | | | | | | | | |
| tests | | | | | | | | | | | | |
| (remote and | | | | | | | | | | | | |
| in-person)²⁸ | | | | | | | | | | | | |
| Questionnaires: | — | — | — | — | X | — | X | X | X | X | X | X |
| Caregiver | | | | | | | | | | | | |
| as | | | | | | | | | | | | |
| Respondent²⁹ | | | | | | | | | | | | |
| Immune/Infusion | X³⁰ <--------------------------------------------------------------------------------------> X | | | — | — | — | — | — | — | — | — | — |
| prophylaxis | | | | | | | | | | | | |
| Steroid | — | — | X³¹ <----------------------------------------------------------------------------------> X | | | | | | | | | — |
| tapering / | | | | | | | | | | | | |
| HPA axis | | | | | | | | | | | | |
| monitoring | | | | | | | | | | | | |
| BBP-812 | — | — | — | — | — | — | — | — | — | — | — | — |
| administration³³ | | | | | | | | | | | | |
| Discharge | — | — | — | — | — | — | — | — | — | — | — | — |
| from | | | | | | | | | | | | |
| inpatient unit | | | | | | | | | | | | |
| Caregiver | X³⁵ <---------------------------------------- ----------------------------------------------------> X | | | | | | — | — | — | — | — | — |
| contact | | | | | | | | | | | | |
| (safety | | | | | | | | | | | | |
| status) | | | | | | | | | | | | |

| | | |
|---|---|---|
| Survival | X <-------------------------------------------------------------------------------- | X |
| status | -------------------------------------------------------------------------------- | |
| | ----------------------------------------------------------> X | |
| Adverse | X <-------------------------------------------------------------------------------- | X |
| events | -------------------------------------------------------------------------------- | |
| | ----------------------------------------------------------> X | |
| Concomitant | X <-------------------------------------------------------------------------------- | X |
| medications | -------------------------------------------------------------------------------- | |
| | ----------------------------------------------------------> X | |

Abbreviations:
AAV9: adeno-associated virus serotype 9;
Abs: antibodies;
AE: adverse event;
ALT: alanine aminotransferase;
ASPA: aspartoacylase;
AST: aspartate aminotransferase;
BUN: blood urea nitrogen;
COVID-19: coronavirus disease-2019;
d: day;
CSF: cerebrospinal fluid;
ECG: electrocardiogram;
eCRF: electronic case report form;
EEG: electroencephalogram;
eGFR: estimated glomerular filtration rate;
ELISA: enzyme-linked immunosorbent assay;
ELISpot: enzyme-linked immunospot;
ET: Early Termination (visit);
GGT: gamma-glutamyl transferase;
HEENT: head, ears, eyes, nose, and throat;
HIV: human immunodeficiency virus;
HPA: hypothalamic-pituitary-adrenal;
IV: intravenous;
IM: intramuscular;
LDH: lactate dehydrogenase;
M: month;
MRI: magnetic resonance imaging;
MRS: magnetic resonance spectroscopy;
NAA: N-acetylaspartic acid;
SARS-CoV-2: severe acute respiratory syndrome coronavirus 2;
SCR: Screening (period);
VEP: visual evoked potential;
W: week
¹Refer to Site Reference Manual for additional details about conducting the assessments.
²As appropriate to the participant's circumstances, assessments may be conducted at the participant's home by a qualified healthcare professional.
³The Screening period will be up to 42 days and will begin when the first Screening assessment is performed. During the Screening Period, a participant's medical records may be used for collection of relevant data (e.g., medical history), as appropriate.
¹Re-screening assessments to be conducted if >90 days have elapsed since initial screening assessment. Obtain informed re-consent.
²During the Baseline period, treatment eligibility of the participant will be confirmed. If the time since the Screening assessment is >42 days (but ≤90 days), then the Investigator and Sponsor will determine which Screening assessments require repeating for confirmation of eligibility and establishing a baseline before BBP-812 dosing.
³Baseline Study procedures are permitted to be conducted over a 10-day period (Days −10 to Day 0 [pre-dose]), except for immune/infusion prophylaxis (refer to Notes 30 and 32), to reduce the physical and/or emotional burden on the participant and family. Baseline laboratory samples must be collected before initiating glucocorticoid prophylaxis. All Baseline assessments must be completed before administration Of BBP-812.
⁴Age, sex, ethnicity, and race.
⁵Medical and surgical history, prior medications and/or physical therapy interventions; Canavan disease history, Canavan disease family history, seizure history.
⁶Confirm treatment eligibility since Screening assessments. Treatment eligibility should be confirmed before conducting Baseline procedures that are invasive and/or require sedation or anesthesia (eg, CSF collection, imaging).
⁷Full physical examination to include body systems: skin, HEENT, respiratory, cardiovascular, gastrointestinal, blood and lymphatic, and musculoskeletal.
⁸Directed physical examination/limited physical assessment to include body systems (at minimum): skin, cardiovascular, respiratory, and gastrointestinal. On Day 0 (Dosing), visually assess the Skin at the infusion site at the same timepoints specified for vital sign collection in Note 11.
⁹Neurological examination to include the following domains: mental status, language, cranial nerves, motor function and musculoskeletal, reflexes, sensory, and coordination.
¹⁰Opthalmologic examination to include VEP testing.
¹¹Blood pressure, pulse, rectal temperature, and respiratory rate. In addition, oxygen saturation (pulse oximetry) will be measured through the Day 14 visit. Vital sign measurements are permitted at other times as clinically indicated. Vital signs will be recorded within 15 minutes before infusion of BBP-812. Once dosing has commenced, vital signs will be recorded every 15 (±5) minutes during the duration of the infusion. After the infusion has ended and the infusion line has been flushed, vital signs will be recorded for a total of 14 hours at the following timepoints: 5 (±1) minutes, 15 (±5) minutes, 30 (±5) minutes, 45 (±5) minutes, 1 hour (±5 minutes), 1.5 hours (±5 minutes), 2 hours (±5 minutes), and, then, every hour (±5 minutes) for the next 12 hours. Thereafter, vital sign assessments will be performed in accordance with local clinical practice until discharge and will be captured in the eCRF.
¹²Growth assessments will include body weight (kg), height/length (cm), and head circumference (cm).
¹³EEG to be performed as close as possible before the date of BBP-812 infusion. Post-dose EEGs are permitted as clinically indicated at the Investigator’s discretion.
¹⁴Collect Baseline laboratory samples before initiating glucocorticoid prophylaxis. Samples for laboratory tests will be based on volumes specified in the Site Reference Manual. Residual volume from laboratory samples may be archived for additional future analyses.
¹⁵Routine clinical laboratory testing will be performed locally from Study Day 1-28, unless otherwise specified by the Sponsor. Hematology assessments: white blood cell count, red blood cell count, hemoglobin, hematocrit, platelet count, differential (neutrophils, eosinophils, lymphocytes, monocytes, and basophils), and peripheral blood smear. Blood chemistry assessments: albumin, albumin/globulin ratio (calculated), alkaline phosphatase, ALT, AST, bilirubin (fractionated and total), BUN, BUN/creatinine ratio (calculated), calcium, carbon dioxide, chloride, creatinine with eGFR, GGT, globulin (calculated), glucose, LDH, potassium, sodium, and total protein. Urinalysis: appearance, pH, specific gravity, protein, glucose, ketones, and microscopic examination of sediment.
¹⁶Routine Clinical laboratory testing will be performed locally from Study Day 1-28. Coagulation: Partial thromboplastin time and prothrombin time/international normalized ratio.
¹⁷Genetic testing for mutations in the ASPA gene for Canavan disease and the ALDOB gene for hereditary fructose intolerance.
¹⁸Biomarker/immunogenicity sample at Screening will be archived for future analysis. Other biomarker assessment timepoints include serum ketones (beta-hydroxybutyrate).
¹⁹Serum sample will be collected and archived for future exploratory analysis which may consist of anti-AAV9 total antibodies and/or other markers of immune or Canavan disease status.
²⁰Immunogenicity assessments will include testing to detect anti-AAV9 neutralizing antibodies.
²¹Immunogenicity assessments will include testing to detect anti-AAV9 total antibodies by ELISA and anti-ASPA total antibodies by ELISA.
²²Immunogenicity assessments will include T-cell reactivity to AAV9 and ASPA by ELISpot.
²³Pathogen screening assessments: SARS-CoV-2, HIV-1, HIV-2, hepatitis B and C, and tuberculosis (by skin test). Any subsequent testing for SARS-CoV-2 will be performed based on the investigational site’s institutional standards and policies.
²⁴Collect samples for blood biodistribution of BBP-812 and vector shedding in urine, feces, and saliva.
²⁵Collect a sample for vector shedding in urine, feces, and/or saliva at Early Termination.
²⁶CSF will be collected by lumbar puncture to measure NAA, protein, glucose, and cell counts. The procedure will be performed under sedation/general anesthesia and a separate informed consent will be required.
²⁷Conduct the Baseline assessment after treatment eligibility has been confirmed.
²⁸Developmental assessments will be performed remotely and in person depending on the specific test and current circumstances as they may impact participant health and safety. Every effort will be made to have the test administered by the same trained Rater to the same participant at the same time of day for consistency in administration.
²⁹The participant's primary caregiver will be asked to provide responses. Every effort will be made to have the test administered by the same trained Rater to the same respondent for consistency in administration.
³⁰For all eligible participants, begin glucocorticoid prophylaxis on Day −1 at least 24 hours before dosing with BBP-812, continue the prophylactic regimen throughout the participant's inpatient stay, and continue the prophylactic regimen for at least the first month after BBP-812 administration.
³¹At the Day 28 visit, determine whether to start glucocorticoid taper. Initiation and management of the glucocorticoid taper, use of stress steroids, and confirmation of HPA axis recovery will be performed.
³²Antihistamine prophylaxis for prevention of infusion reactions.
³³Administration of BBP-812 must occur only after all Baseline assessments have been completed.
³⁴Participants will remain in the unit for safety observation for at least 72 hours after completion of BBP-812 dosing or longer as medically indicated per Investigator judgment.
³⁵Participant's caregiver will be contacted weekly to determine participant's safety status when such contact does not coincide with a scheduled study visit.
³⁶During the Screening and Baseline Periods (from time informed consent is signed up to the time of BBP-812 infusion) any clinically significant changes in the participant's health will be recorded as AEs.

1. Refer to the Site Reference Manual for additional details about conducting the assessments.
2. As appropriate to the participant's circumstances, assessments may be conducted at the participant's home by a qualified healthcare professional.
3. The Screening period will be up to 42 days and will begin when the first Screening assessment is performed. During the Screening Period, a participant's medical records may be used for collection of relevant data (e.g., medical history), as appropriate.
1. Re-screening assessments to be conducted if >90 days have elapsed since initial screening assessment. Obtain informed re-consent.
2. During the Baseline period, treatment eligibility of the participant will be confirmed. If the time since the Screening assessment is >42 days (but <90 days), then the Investigator and Sponsor will determine which Screening assessments require repeating for confirmation of eligibility and for establishing a baseline before BBP-812 dosing.
3. Baseline study procedures are permitted to be conducted over a 10-day period (Days -10 to Day 0 [pre-dose]), except for immune/infusion prophylaxis (refer to Notes 30 and 32), to reduce the physical and/or emotional burden on the participant and family. Baseline laboratory samples must be collected before initiating glucocorticoid prophylaxis. All Baseline assessments must be completed before administration of BBP-812.
4. Age, sex, ethnicity, and race.
5. Medical and surgical history, prior medications and/or physical therapy interventions; Canavan disease history, Canavan disease family history, seizure history.
6. Confirm treatment eligibility since Screening assessments. Treatment eligibility should be confirmed before conducting Baseline procedures that are invasive and/or require sedation or anesthesia (eg, CSF collection, imaging).
7. Full physical examination to include body systems: skin, HEENT, respiratory, cardiovascular, gastrointestinal, blood and lymphatic, and musculoskeletal.
8. Directed physical examination/limited physical assessment to include body systems (at minimum): skin, cardiovascular, respiratory, and gastrointestinal. On Day 0 (Dosing), visually assess the skin at the infusion site at the same timepoints specified for vital sign collection in Note 11.
9. Neurological examination to include the following domains: mental status, language, cranial nerves, motor function and musculoskeletal, reflexes, sensory, and coordination.
10. Ophthalmologic examination to include VEP testing.
11. Blood pressure, pulse, rectal temperature, and respiratory rate. In addition, oxygen saturation (pulse oximetry) will be measured through the Day 14 visit. Vital sign measurements are permitted at other times as clinically indicated. Vital signs will be recorded within 15 minutes before infusion of BBP-812. Once dosing has commenced, vital signs will be recorded every 15 (±5) minutes during the duration of the infusion. After the infusion has ended and the infusion line has been flushed, vital signs will be recorded for at total of 14 hours at the following timepoints: 5 (±1) minutes, 15 (±5) minutes, 30 (±5) minutes, 45 (±5) minutes, 1 hour (±5 minutes), 1.5 hours (±5 minutes), 2 hours (±5 minutes), and, then, every hour (±5 minutes) for the next 12 hours. Thereafter, vital sign assessments will be performed in accordance with local clinical practice until discharge and will be captured in the eCRF.
12. Growth assessments will include body weight (kg), height/length (cm), and head circumference (cm).
13. EEG to be performed as close as possible before the date of BBP-812 infusion. Post-dose EEGs are permitted as clinically indicated at the Investigator's discretion.
14. Collect Baseline laboratory samples before initiating glucocorticoid prophylaxis. Samples for laboratory tests will be based on volumes specified in the Site Reference Manual. Residual volume from laboratory samples may be archived for additional future analyses.
15. Routine clinical laboratory testing will be performed locally from Study Day 1-28, unless otherwise specified by the Sponsor. Hematology assessments: white blood cell count, red blood cell count, hemoglobin, hematocrit, platelet count, differential (neutrophils, eosinophils, lymphocytes, monocytes, and basophils), and peripheral blood smear. Blood chemistry assessments: albumin, albumin/globulin ratio (calculated), alkaline phosphatase, ALT, AST, bilirubin (fractionated and total), BUN, BUN/creatinine ratio (calculated), calcium, carbon dioxide, chloride, creatinine with eGFR, GGT, globulin (calculated), glucose, LDH, potassium, sodium, and total protein. Urinalysis: appearance, pH, specific gravity, protein, glucose, ketones, and microscopic examination of sediment.
16. Routine clinical laboratory testing will be performed locally from Study Day 1-28. Coagulation: Partial thromboplastin time and prothrombin time/international normalized ratio.
17. Genetic testing for mutations in the ASPA gene for Canavan disease and the ALDOB gene for hereditary fructose intolerance.
18. Biomarker/immunogenicity sample at Screening will be archived for future analysis. Other biomarker assessment timepoints include serum ketones (beta-hydroxybutyrate).
19. Serum sample will be collected and archived for future exploratory analysis which may consist of anti-AAV9 total antibodies and/or other markers of immune or Canavan disease status.
20. Immunogenicity assessments will include testing to detect anti-AAV9 neutralizing antibodies.
21. Immunogenicity assessments will include testing to detect anti-AAV9 total antibodies by ELISA and anti-ASPA total antibodies by ELISA.
22. Immunogenicity assessments will include T-cell reactivity to AAV9 and ASPA by ELISpot.
23. Pathogen screening assessments: SARS-CoV-2, HIV-1, HIV-2, hepatitis B and C, and tuberculosis (by skin test). Any subsequent testing for SARS-CoV-2 will be performed based on the investigational site's institutional standards and policies.
24. Collect samples for blood biodistribution of BBP-812 and vector shedding in urine, feces, and saliva.
25. Collect a sample for vector shedding in urine, feces, and/or saliva at Early Termination.
26. CSF will be collected by lumbar puncture to measure NAA, protein, glucose, and cell counts. The procedure will be performed under sedation/general anesthesia and a separate informed consent will be required.
27. Conduct the Baseline assessment after treatment eligibility has been confirmed.
28. Developmental assessments will be performed remotely and in person depending on the specific test and current circumstances as they may impact participant health and safety. Every effort will be made to have the test administered by the same trained Rater to the same participant at the same time of day for consistency in administration.
29. The participant's primary caregiver will be asked to provide responses. Every effort will be made to have the test administered by the same trained Rater to the same respondent for consistency in administration.
30. For all eligible participants, begin glucocorticoid prophylaxis on Day -1 at least 24 hours before dosing with BBP-812, continue the prophylactic regimen throughout the participant's inpatient stay, and continue the prophylactic regimen for at least the first month after BBP-812 administration.
31. At the Day 28 visit, determine whether to start glucocorticoid taper. Initiation and management of the glucocorticoid taper, use of stress steroids, and confirmation of HPA axis recovery will be performed.
32. Antihistamine prophylaxis for prevention of infusion reactions.
33. Administration of BBP-812 must occur only after all Baseline assessments have been completed.
34. Participants will remain in the unit for safety observation for at least 72 hours after completion of BBP-812 dosing or longer as medically indicated per Investigator judgment.
35. Participant's caregiver will be contacted weekly to determine participant's safety status when such contact does not coincide with a scheduled study visit.
36. During the Screening and Baseline Periods (from time informed consent is signed up to the time of BBP-812 infusion) any clinically significant changes in the participant's health will be recorded as AEs.

TABLE 14
Study Schedule of Events: Long-Term Follow-Up Period

| | Long-Term Follow-Up Period | | | | | | | | | | | | |
|---|---|---|---|---|---|---|---|---|---|---|---|---|---|
| | Study Month (M) (±1 week) | | | | | | | | | | | | ET |
| Study Assessment^{1, 2} | M 15 | M 18 | M 21 | M 24 | M 27 | M 30 | M 33 | M 36 | M 42 | M 48 | M 54 | M 60 | (±3 d) |
| Physical examination (full)³ | X | X | X | X | — | X | — | X | X | X | X | X | X |
| Neurological examination | X | X | X | X | — | X | — | X | X | X | X | X | X |
| Ophthalmologic examination⁴ | X | X | X | X | — | X | — | X | X | X | X | X | X |
| Vital signs⁵ | X | X | X | X | — | X | — | X | X | X | X | X | X |
| Growth assessments⁶ | X | X | X | X | — | X | — | X | X | X | X | X | X |
| 12-lead ECG | X | X | X | X | — | X | — | X | X | X | X | X | X |
| Electroencephalogram (EEG)⁷ | — | — | — | — | — | — | — | — | — | — | — | — | — |
| Laboratory assessments⁸ | — | — | — | — | — | — | — | — | — | — | — | — | — |
| Routine clinical laboratory tests⁹ | X | X | X | X | — | X | — | X | X | X | X | X | X |
| Coagulation¹⁰ | X | X | X | X | — | X | — | X | X | X | X | X | X |
| Urine NAA | X | X | X | X | — | X | — | X | X | X | X | X | X |
| Biomarkers¹¹ | X | X | X | X | — | X | — | X | X | X | X | X | X |
| Immunogenicity: neutralizing | X | X | X | X | — | X | — | X | X | X | X | X | X |
| antibodies¹² | | | | | | | | | | | | | |
| Immunogenicity: total ¹³ | X | X | X | X | — | X | — | X | X | X | X | X | X |
| Immunogenicity: T-cell | X | X | X | X | — | X | — | X | X | X | X | X | X |
| reactivity¹⁴ | | | | | | | | | | | | | |
| Blood biodistribution¹⁵ | X | X | X | X | — | X | — | X | X | X | X | X | X |
| Vector shedding¹⁵ | X | X | X | X | — | X | — | X | X | X | X | X | X¹⁶ |
| Cerebrospinal fluid¹⁷ | — | X | — | X | — | X | — | X | X | X | X | X | X |
| Imaging: brain MRI | — | X | — | X | — | X | — | X | X | X | X | X | X |
| Imaging: brain MRS | — | X | — | X | — | X | — | X | X | X | X | X | X |
| Administered developmental tests | X¹⁹ | X¹⁹ | X¹⁹ | X¹⁹ | X¹⁹ | X¹⁹ | X¹⁹ | X¹⁹ | X¹⁹ | X¹⁹ | X¹⁹ | X¹⁹ | X |
| (remote and in-person)¹⁸ | | | | | | | | | | | | | |
| Questionnaires: caregiver as | X | X | X | X | X | X | X | X | X | X | X | X | X |
| respondent²⁰ | | | | | | | | | | | | | |

| | | |
|---|---|---|
| Survival status | X <-----------------------------------------------------------------------------------------------------------------------------------------> X | X |
| Serious adverse events | X <-----------------------------------------------------------------------------------------------------------------------------------------> X | X |
| Concomitant medications | X <-----------------------------------------------------------------------------------------------------------------------------------------> X | X |

Abbreviations:
AAV9: adeno-associated virus serotype 9;
Abs: antibodies;
ALT: alanine aminotransferase;
ASPA: aspartoacylase;
AST: aspartate aminotransferase;
BUN: blood urea nitrogen;
CSF: cerebrospinal fluid;
d: day;
ECG: electrocardiogram;
EEG: electroencephalogram;
eGFR: estimated glomerular filtration rate;
ELISA: enzyme-linked immunosorbent assay;
ELISpot: enzyme-linked immunospot;
ET: Early Termination (visit);
GGT: gamma-glutamyl transferase;
HEENT: head, ears, eyes, nose and throat;
LDH: lactate dehydrogenase;
M: month;
MRI: magnetic resonance imaging;
MRS: magnetic resonance spectroscopy;
NAA: N-acetylaspartic acid;
SARS-CoV-2: severe acute respiratory syndrome coronavirus 2;
VEP: visual evoked potential
¹Refer to the Site Reference Manual for additional details about conducting the assessments.
²As appropriate to the participant's circumstances, assessments may be conducted at the participant's home by a qualified healthcare professional.
³Full physical examination to include body systems: skin, HEENT, respiratory, cardiovascular, gastrointestinal, blood and lymphatic, and musculoskeletal.
⁴Ophthalmologic examination to include VEP testing.
⁵Blood pressure, pulse, rectal temperature, and respiratory rate. Vital sign measurements are permitted at other times as clinically indicated.
⁶Growth assessments will include body weight (kg), height/length (cm), and head circumference (cm).
⁷EEG is permitted post-dose as clinically indicated at the Investigator's discretion.
⁸Samples for laboratory tests will be based on volumes specified in the Site Reference Manual. Residual volume from laboratory samples may be archived for additional future analyses.
⁹Hematology assessments: white blood cell count, red blood cell count, hemoglobin, hematocrit, platelet count, differential (neutrophils, eosinophils, lymphocytes, monocytes, and basophils), and peripheral blood smear. Blood chemistry assessments: albumin, albumin/globulin ratio (calculated), alkaline phosphatase, ALT, AST, bilirubin (fractionated and total), BUN, BUN/creatinine ratio (calculated), calcium, carbon dioxide, chloride, creatinine with eGFR, GGT, globulin (calculated), glucose, LDH, potassium, sodium, and total protein. Urinalysis: appearance, pH, specific gravity, protein, glucose, ketones, and microscopic examination of sediment.
¹⁰Coagulation: Partial thromboplastin time and prothrombin time/international normalized ratio.
¹¹Biomarker assessment includes serum ketones (beta-hydroxybutyrate).
¹²Immunogenicity assessments will include testing to detect anti-AAV9 neutralizing antibodies.
¹³ Immunogenicity assessments will include testing to detect anti-AAV9 total antibodies by ELISA and anti-ASPA total antibodies by ELISA.
¹⁴Immunogenicity assessments will include T-cell reactivity to AAV9 and ASPA by ELISpot.
¹⁵Continue sample collection for blood biodistribution and vector shedding in urine, feces, and saliva.
¹⁶Collect sample for vector shedding in urine, feces, and saliva at Early Termination.
¹⁷CSF will be collected by lumbar puncture to measure NAA, protein, glucose, and cell counts The procedure will be performed under sedation/general anesthesia and separate informed consent will be required.
¹⁸Developmental assessments will be performed remotely and in person depending on the specific test and current circumstances as they may impact participant health and safety. Every effort will be made to have the test administered by the same trained Rater to the same participant at the same time of day for consistency in administration.
¹⁹Assessments during the Long-Term Follow-Up Years 3, 4, and 5 will be conducted every 6 months via in-person (circumstances permitting) and remote visits.
²⁰The participant's primary caregiver will be asked to provide responses. Every effort will be made to have the test administered by the same trained Rater to the same respondent for consistency in administration.

1. Refer to the Site Reference Manual for additional details about conducting the assessments.
2. As appropriate to the participant's circumstances, assessments may be conducted at the participant's home by a qualified healthcare professional.
3. Full physical examination to include body systems: skin, HEENT, respiratory, cardiovascular, gastrointestinal, blood and lymphatic, and musculoskeletal.
4. Ophthalmologic examination to include VEP testing.
5. Blood pressure, pulse, rectal temperature, and respiratory rate. Vital sign measurements are permitted at other times as clinically indicated.
6. Growth assessments will include body weight (kg), height/length (cm), and head circumference (cm).
7. EEG is permitted post-dose as clinically indicated at the Investigator's discretion.
8. Samples for laboratory tests will be based on volumes specified in the Site Reference Manual. Residual volume from laboratory samples may be archived for additional future analyses.
9. Hematology assessments: white blood cell count, red blood cell count, hemoglobin, hematocrit, platelet count, differential (neutrophils, eosinophils, lymphocytes, monocytes, and basophils), and peripheral blood smear. Blood chemistry assessments: albumin, albumin/globulin ratio (calculated), alkaline phosphatase, ALT, AST, bilirubin (fractionated and total), BUN, BUN/creatinine ratio (calculated), calcium, carbon dioxide, chloride, creatinine with eGFR, GGT, globulin (calculated), glucose, LDH, potassium, sodium, and total protein. Urinalysis: appearance, pH, specific gravity, protein, glucose, ketones, and microscopic examination of sediment.
10. Coagulation: Partial thromboplastin time and prothrombin time/international normalized ratio.
11. Biomarker assessment includes serum ketones (beta-hydroxybutyrate).
12. Immunogenicity assessments will include testing to detect anti-AAV9 neutralizing antibodies.
13. Immunogenicity assessments will include testing to detect anti-AAV9 total antibodies by ELISA and anti-ASPA total antibodies by ELISA.
14. Immunogenicity assessments will include T-cell reactivity to AAV9 and ASPA by ELISpot.
15. Continue sample collection for blood biodistribution and vector shedding in urine, feces, and saliva.
16. Collect sample for vector shedding in urine, feces, and saliva at Early Termination.
17. CSF will be collected by lumbar puncture to measure NAA, protein, glucose, and cell counts The procedure will be performed under sedation/general anesthesia and separate informed consent will be required.
18. Developmental assessments will be performed remotely and in person depending on the specific test and current circumstances as they may impact participant health and safety. Every effort will be made to have the test administered by the same trained Rater to the same participant at the same time of day for consistency in administration.
19. Assessments during the Long-Term Follow-Up Years 3, 4, and 5 will be conducted every 6 months via in-person (circumstances permitting) and remote visits.
20. The participant's primary caregiver will be asked to provide responses. Every effort will be made to have the test administered by the same trained Rater to the same respondent for consistency in administration.

### Example 4: Preliminary results of the Phase 1/2 Open Label Clinical Trial

Two patients were dosed with BBP-812 as described in Example 3. Robust and durable post-treatment decreases in N-acetylaspartate (NAA) in urine, cerebrospinal fluid (CSF), and brain tissue were observed using magnetic resonance spectroscopy (MRS) imaging.

Signs of new myelination measured with magnetic resonance imaging (MRI) were also observed. Reduction in brain NAA is an early signal that suggests that intravenous (IV) administered BBP-812 has reached its intended target behind the blood-brain-barrier and is expressing functional aspartoacylase (ASPA) enzyme. There is evidence in the scientific literature that lower NAA levels are associated with milder disease. More time will be needed to see how those reductions in NAA translate to clinical outcomes.

IV infusions of BBP-812 have been well-tolerated, and to date, no participants have experienced a treatment-related serious adverse event. At month 6 post-treatment, Participant 1 showed a 77% lowering of NAA in the CSF, a 15% reduction in NAA in brain white matter measured by MRS imaging, and a -50% decrease in urine NAA. At month 3 post-treatment, Participant 2 showed an 89% reduction of NAA in the CSF, a greater than 50% decrease in NAA in brain white matter measured by MRS imaging, and an 81% drop in urine NAA. These observed biochemical changes suggest that BBP-812 is reaching cells critical to the Canavan disease process, a milestone in this disease. These data represent preliminary results, and the final safety and efficacy profile of the investigational gene therapy remains to be fully established.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited.

However, mention of any reference, article, publication, patent, patent publication, and patent application cited herein is not, and should not be taken as an acknowledgment or any form of suggestion that they constitute valid prior art or form part of the common general knowledge in any country in the world.

The embodiments illustrated and discussed in this specification are intended only to teach those skilled in the art the best way known to the inventors to make and use the invention.

Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

### Enumerated Embodiments

Embodiment 1. A method, comprising: administering to a subject a therapeutically effective amount of a recombinant adeno-associated virus (rAAV) vector, wherein the rAAV vector comprises:
   (i) a nucleic acid molecule comprising at least one AAV inverted terminal repeat (ITR) and
   (ii) a non-AAV nucleotide sequence encoding aspartoacylase (ASPA), wherein the non-AAV nucleotide sequence is operably linked to a promoter; and wherein the therapeutically effective amount is in the range of about 10¹³ vg/kg to about 10¹⁵ vg/kg.
Embodiment 2. A method of expressing aspartoacylase (ASPA) in a subject in need thereof, comprising: administering to the subject a therapeutically effective amount of a recombinant adeno-associated virus (rAAV) vector, wherein the rAAV vector comprises:
   (i) a nucleic acid molecule comprising at least one AAV inverted terminal repeat (ITR) and
   (ii) a non-AAV nucleotide sequence encoding ASPA, wherein the non-AAV nucleotide sequence is operably linked to a promoter; and wherein the therapeutically effective amount is in the range of about 10¹³ vg/kg to about 10¹⁵ vg/kg, thereby expressing ASPA in the subject.
Embodiment 3. A method of increasing the level of aspartoacylase (ASPA) in a subject in need thereof, comprising: administering to the subject a therapeutically effective amount of a recombinant adeno-associated virus (rAAV) vector, wherein the rAAV vector comprises:
   (i) a nucleic acid molecule comprising at least one AAV inverted terminal repeat (ITR) and
   (ii) a non-AAV nucleotide sequence encoding ASPA, wherein the non-AAV nucleotide sequence is operably linked to a promoter; and wherein the therapeutically effective amount is in the range of about 10¹³ vg/kg to about 10¹⁵ vg/kg, thereby increasing the level of ASPA in the subject.
Embodiment 4. The method of any one of embodiments 1-3, wherein the subject is in need of expression of ASPA.
Embodiment 5. The method of any one of embodiments 1-4, wherein the subject has a reduced level and/or function of ASPA.
Embodiment 6. The method of any one of embodiments 1-5, wherein the subject has leukodystrophy.
Embodiment 7. A method of treating leukodystrophy in a subject in need thereof, comprising: administering to the subject a therapeutically effective amount of a recombinant adeno-associated virus (rAAV) vector, wherein the rAAV vector comprises:
   (i) a nucleic acid molecule comprising at least one AAV inverted terminal repeat (ITR) and
   (ii) a non-AAV nucleotide sequence encoding ASPA, wherein the non-AAV nucleotide sequence is operably linked to a promoter; and wherein the therapeutically effective amount is in the range of about 10¹³ vg/kg to about 10¹⁵ vg/kg, thereby treating leukodystrophy in the subject.
Embodiment 8. The method of embodiment 6 or embodiment 7, wherein the leukodystrophy is associated with a condition selected from the group consisting of Canavan disease, adrenomyeloneuropathy, Alexander disease, cerebrotendinous xanthomatosis, Krabbe disease, metachromatic leukodystrophy, adrenoleukodystrophy, Pelizaeus-Merzbacher disease, and Refsum disease.
Embodiment 9. The method of embodiment 8, wherein the leukodystrophy is associated with Canavan disease.
Embodiment 10. The method of any one of embodiments 6-9, further comprising selecting a subject with leukodystrophy prior to administering the rAAV vector.
Embodiment 11. A method of treating Canavan disease in a subject in need thereof, comprising: administering to the subject a therapeutically effective amount of a recombinant adeno-associated virus (rAAV) vector, wherein the rAAV vector comprises:
   (i) a nucleic acid molecule comprising at least one AAV inverted terminal repeat (ITR) and
   (ii) a non-AAV nucleotide sequence encoding ASPA, wherein the non-AAV nucleotide sequence is operably linked to a promoter; and wherein the therapeutically effective amount is in the range of about 10¹³ vg/kg to about 10¹⁵ vg/kg, thereby treating Canavan disease in the subject.
Embodiment 12. The method of embodiment 11, further comprising selecting a subject with Canavan disease prior to administering the rAAV vector.
Embodiment 13. The method of any one of embodiments 1-12, wherein administration of the rAAV vector results in expression of ASPA in a tissue of the subject.
Embodiment 14. The method of embodiment 13, wherein the tissue is peripheral tissue or central nervous system (CNS) tissue.
Embodiment 15. The method of any one of embodiments 1-14, wherein it has been determined that there is a metabolic imbalance comprising a shift from glycolysis to beta-oxidation in the subject.
Embodiment 16. The method of embodiment 15, wherein the method further comprises detecting the metabolic imbalance by evaluating levels of one or more glycolysis and/or beta-oxidation factors.
Embodiment 17. The method of embodiment 16, wherein the levels of one or more glycolysis and/or beta-oxidation factors are evaluated using central nervous system (CNS) fluid obtained from the subject.
Embodiment 18. The method of any one of embodiments 15-17, wherein the method further comprises:
   (a) obtaining CNS fluid from the subject;
   (b) detecting increased beta-oxidation in the CNS fluid; and
   (c) based on the detection in (b), administering the rAAV to the subject.
Embodiment 19. The method of any one of embodiments 15-18, wherein the method further comprises: (a) measuring a metabolic profile of a biological sample obtained from the subject; and (b) identifying a metabolic imbalance comprising a shift from glycolysis to beta-oxidation based upon the metabolic profile.
Embodiment 20. The method of embodiment 19, wherein measuring the metabolic profile comprises assaying the biological sample using liquid chromatography (LC), mass spectrometry (MS), liquid chromatography/mass spectrometry (LC/MS), or Ultrahigh Performance Liquid Chromatography-Tandem Mass Spectroscopy (UPLC-MS/MS).
Embodiment 21. The method of embodiment 19 or embodiment 20, wherein the biological sample comprises blood, serum, CNS tissue or cerebrospinal fluid (CSF).
Embodiment 22. The method of embodiment 21, wherein the CNS tissue is brain tissue.
Embodiment 23. The method of any one of embodiments 19-22, wherein the metabolic profile comprises a level of a first biomarker selected from the group consisting of glucose, glucose-6-phosphate, 3-phosphoglycerate, pyruvate, lactate, and phosphoenolpyruvate.
Embodiment 24. The method of any one of embodiments 19-23, wherein the metabolic profile comprises a level of a second biomarker selected from the group consisting of carnitine, malonylcarnitine, myristoylcarnitine, palmitoylcarnitine, malonylcarnitine, and beta-hydroxybutyrate.
Embodiment 25. The method of any one of embodiments 1-24, wherein the therapeutically effective amount is in the range of about 10¹⁴ vg/kg to about 5×10¹⁴ vg/kg.

Embodiment 26. The method of embodiment 25, wherein the therapeutically effective amount is about 1.32×10¹⁴ vg/kg.
Embodiment 27. The method of embodiment 25, wherein the therapeutically effective amount is about 3×10¹⁴ vg/kg.
Embodiment 28. The method of any one of embodiments 1-27, wherein the rAAV is administered via infusion.
Embodiment 29. The method of embodiment 28, wherein the rAAV is administered via intravenous infusion.
Embodiment 30. The method of any one of embodiments 1-27, wherein the rAAV is administered via injection.
Embodiment 31. The method of embodiment 30, wherein the injection is selected from the group consisting of intravenous injection, intravascular injection, and intraventricular injection.
Embodiment 32. The method of any one of embodiments 1-31, wherein the subject is less than, or equal to, 30 months of age.
Embodiment 33. The method of any one of embodiments 1-32, wherein ASPA comprises human ASPA protein.
Embodiment 34. The method of any one of embodiments 1-33, wherein ASPA comprises an amino acid sequence of SEQ ID NO: 6, or an amino acid sequence with at least about 95%, at least about 96%, at least about 97%, at least about 98% or at least about 99% identity to SEQ ID NO: 6.
Embodiment 35. The method of any one of embodiments 1-34, wherein the promoter is an astrocyte-specific promoter, a glial fibrillary acidic protein (GFAP) promoter, or an enhanced chicken R-actin promoter.
Embodiment 36. The method of any one of embodiments 1-35, wherein the promoter is a cytomegalovirus/0-actin hybrid promoter, or a PGK promoter.
Embodiment 37. The method of embodiment 36, wherein the cytomegalovirus/0-actin hybrid promoter is a CAG promoter, a CB6 promoter, or a CBA promoter.
Embodiment 38. The method of any one of embodiments 1-37, wherein the non-AAV nucleotide sequence encoding ASPA comprises or consists of the human ASPA cDNA.
Embodiment 39. The method of any one of embodiments 1-38, wherein the non-AAV nucleotide sequence encoding ASPA comprises or consists of a codon-optimized nucleotide sequence.
Embodiment 40. The method of any one of embodiments 1-39, wherein the non-AAV nucleotide sequence encoding ASPA comprises or consists of SEQ ID NO: 1.
Embodiment 41. The method of any one of embodiments 1-40, wherein the non-AAV nucleotide sequence encodes the amino acid sequence of SEQ ID NO: 6, or an amino acid sequence with at least about 95%, at least about 96%, at least about 97%, at least about 98% or at least about 99% identity to SEQ ID NO: 6.
Embodiment 42. The method of any one of embodiments 1-41, wherein the nucleic acid molecule comprises a cytomegalovirus immediate-early enhancer.
Embodiment 43. The method of any one of embodiments 1-42, wherein the nucleic acid molecule comprises a rabbit β-globin polyA signal.
Embodiment 44. The method of any one of embodiments 1-43, wherein the nucleic acid molecule comprises a Kozak sequence.
Embodiment 45. The method of any one of embodiments 1-44, wherein the nucleic acid molecule comprises an miR-122 binding site.
Embodiment 46. The method of any one of embodiments 1-45, wherein the ITR is an AAV1, AAV2, AAV3, AAV4, AAV5, AAV6, AAV7, AAV8, AAV9, AAV10, AAV11, AAV12, rh10, or rh74 serotype ITR.
Embodiment 47. The method of any one of embodiments 1-46, wherein the rAAV is an AAV1, AAV2, AAV3, AAV4, AAV5, AAV6, AAV7, AAV8, AAV9, AAV10, AAV11, AAV12, rh10, or rh74 serotype rAAV.
Embodiment 48. The method of any one of embodiments 1-47, wherein the rAAV is an AAV9 serotype rAAV.
Embodiment 49. The method of any one of embodiments 1-48, wherein the rAAV is a self-complementary rAAV (scAAV).
Embodiment 50. The method of any one of embodiments 1-49, wherein the rAAV is a single-stranded rAAV (ssAAV).
Embodiment 51. A method, comprising:
   administering to a subject about 1.32×10¹⁴ vg/kg of a recombinant self-complementary adeno-associated virus 9 (scAAV9) vector via intravenous infusion, wherein the rAAV9 vector comprises:
   (i) a nucleic acid molecule comprising at least one AAV inverted terminal repeat (ITR), a cytomegalovirus immediate-early enhancer, a Kozak sequence, and a rabbit β-globin polyA signal; and
   (ii) a non-AAV nucleotide sequence encoding the amino acid sequence of SEQ ID NO: 1, wherein the non-AAV nucleotide sequence is operably linked to CB6 promoter that directs expression of ASPA in the subject, wherein the subject is less than, or equal to, 30 months of age, and wherein the subject has Canavan disease.
Embodiment 52. A method, comprising:
   administering to a subject about 3×10¹⁴ vg/kg of a recombinant self-complementary adeno-associated virus 9 (scAAV9) vector via intravenous infusion, wherein the rAAV9 vector comprises:
   (i) a nucleic acid molecule comprising at least one AAV inverted terminal repeat (ITR), a cytomegalovirus immediate-early enhancer, a Kozak sequence, and a rabbit β-globin polyA signal; and
   (ii) a non-AAV nucleotide sequence encoding the amino acid sequence of SEQ ID NO: 1, wherein the non-AAV nucleotide sequence is operably linked to CB6 promoter that directs expression of ASPA in the subject, wherein the subject is less than, or equal to, 30 months of age, and wherein the subject has Canavan disease.
Embodiment 53. The method of any one of embodiments 1-52, further comprising: (a) administering a small molecule metabolic modulator to the subject; (b) prescribing to the subject a dietary intervention, wherein the dietary intervention promotes glycolysis and/or reduces beta-oxidation in the subject; and/or (c) administering an immune-suppressing agent to the subject.
Embodiment 54. The method of any one of embodiments 1-53, further comprising administering a therapeutically effective amount of a glucocorticoid to the subject.
Embodiment 55. The method of embodiment 54, wherein the glucocorticoid is administered before, concurrently with, and/or after administration of the rAAV vector.
Embodiment 56. The method of embodiment 54 or embodiment 55, wherein the glucocorticoid is prednisolone, methylprednisolone, or a combination thereof.
Embodiment 57. The method of any one of embodiments 1-56, further comprising administering a therapeutically effective amount of an anti-histamine to the subject.
Embodiment 58. The method of embodiment 57, wherein the anti-histamine is administered before, concurrently with, and/or after administration of the rAAV vector.
Embodiment 59. The method of embodiment 57 or embodiment 58, wherein the anti-histamine is diphenhydramine, hydroxyzine, chlorpheniramine, or any combination thereof.
Embodiment 60. The method of any one of embodiments 1-59, wherein after said administering, N-acetylaspartate (NAA) levels in urine, cerebrospinal fluid (CSF), and/or brain tissue are decreased.
Embodiment 61. The method of embodiment 60, wherein after said administering, NAA levels in urine are decreased.
Embodiment 62. The method of embodiment 61, wherein said NAA levels in urine are decreased at least about 10%, 15%, 20%, 30%, 40%, 50%, 60%, 70%, 80%, 90%, or greater.
Embodiment 63. The method of embodiment 61 or 62, wherein said NAA levels in urine remain reduced relative to pre-treatment levels for at least 6 months, 9 months, 12 months, 18 months, 24 months, or longer.
Embodiment 64. The method of any one of embodiments 60-63, wherein after said administering, NAA levels in CSF are decreased.
Embodiment 65. The method of embodiment 64, wherein said NAA levels in CSF are decreased at least about 10%, 15%, 20%, 30%, 40%, 50%, 60%, 70%, 80%, 90%, or greater.
Embodiment 66. The method of embodiment 64 or 65, wherein said NAA levels in CSF remain reduced relative to pre-treatment levels for at least 6 months, 9 months, 12 months, 18 months, 24 months, or longer.
Embodiment 67. The method of any one of embodiments 60-66, wherein after said administering NAA, levels in brain tissue are decreased.
Embodiment 68. The method of embodiment 67, wherein said NAA levels in brain tissue are decreased at least about 10%, 15%, 20%, 30%, 40%, 50%, 60%, 70%, 80%, 90%, or greater.
Embodiment 69. The method of embodiment 67 or 68, wherein said NAA levels in brain tissue remain reduced relative to pre-treatment levels for at least 6 months, 9 months, 12 months, 18 months, 24 months, or longer.
Embodiment 70. The method of any one of embodiments 1-69, wherein after said administering, new myelination is observable using magnetic resonance imaging (MRI).

1-10. (canceled)

11. A method of treating Canavan disease in a subject in need thereof, comprising: administering to the subject a therapeutically effective amount of a recombinant adeno-associated virus (rAAV) vector, wherein the rAAV vector comprises:
(i) a nucleic acid molecule comprising at least one AAV inverted terminal repeat (ITR) and
(ii) a non-AAV nucleotide sequence encoding aspartoacylase (ASPA), wherein the non-AAV nucleotide sequence is operably linked to a promoter; and
wherein the therapeutically effective amount is in the range of about 10¹³ vg/kg to about 10¹⁵ vg/kg,
thereby treating Canavan disease in the subject.

12. (canceled)

13. The method of claim 11, wherein administration of the rAAV vector results in expression of ASPA in a peripheral tissue and/or central nervous system (CNS) tissue of the subject.

14. (canceled)

15. The method of claim 11, wherein the subject has it a metabolic imbalance comprising a shift from glycolysis to beta-oxidation, wherein the metabolic imbalance comprises an imbalance in a level of glucose, glucose-6-phosphate, 3-phosphoglycerate, pyruvate, lactate, phosphoenolpyruvate, carnitine, malonylcarnitine, myristoylcarnitine, palmitoylcarnitine, malonylcarnitine, beta-hydroxybutyrate, or a combination thereof.

16-24. (canceled)

25. The method of claim 11, wherein the therapeutically effective amount is in the range of about 1×10¹⁴ vg/kg to about 5×10¹⁴ vg/kg.

26. The method of claim 25, wherein the therapeutically effective amount is about 1.32 X_10¹⁴ vg/kg or about 3×10¹⁴ vg/kg.

27. (canceled)

28. The method of claim 11, wherein the rAAV vector is administered via intravenous infusion, intravenous injection, intravascular injection, or intraventricular injection.

29-31. (canceled)

32. The method of claim 11, wherein the subject is less than, or equal to, 30 months of age.

33. The method of claim 11, wherein ASPA comprises human ASPA protein.

34. The method of claim 11, wherein ASPA comprises an amino acid sequence of SEQ ID NO: 2, or an amino acid sequence with at least 95%, at least 96%, at least 97%, at least 98% or at least 99% identity to SEQ ID NO: 2.

35. The method of claim 11, wherein the promoter is an astrocyte-specific promoter, a glial fibrillary acidic protein (GFAP) promoter, an enhanced chicken R-actin promoter, a cytomegalovirus/3-actin hybrid promoter, or a PGK promoter.

36. (canceled)

37. The method of claim 35, wherein the cytomegalovirus/0-actin hybrid promoter is a CAG promoter, a CB6 promoter, or a CBA promoter.

38. The method of claim 11, wherein the non-AAV nucleotide sequence encoding ASPA comprises or consists of ii)_the human ASPA cDNA; (ii) a codon-optimized nucleotide sequence; and/or iii) SEQ ID NO: 1.

39. (canceled)

40. (canceled)

41. The method of claim 11, wherein the non-AAV nucleotide sequence encodes the amino acid sequence of SEQ ID NO: 6, or an amino acid sequence with at least 95%, at least 96%, at least 97%, at least 98% or at least 99% identity to SEQ ID NO: 6.

42. The method of claim 11, wherein the nucleic acid molecule comprises a cytomegalovirus immediate-early enhancer, a rabbit (-globin polyA signal, a Kozak sequence, and/or an miR-122 binding site.

43-45. (canceled)

46. The method of claim 11, wherein the ITR is an AAV1, AAV2, AAV3, AAV4, AAV5, AAV6, AAV7, AAV8, AAV9, AAV10, AAV11, AAV12, rh10, or rh74 serotype ITR.

47. The method of claim 11, wherein the rAAV is an AAV1, AAV2, AAV3, AAV4, AAV5, AAV6, AAV7, AAV8, AAV9, AAV10, AAV11, AAV12, rh10, or rh74 serotype rAAV.

48. (canceled)

49. The method of claim 11, wherein the rAAV is a self-complementary rAAV (scAAV) and/or a single-stranded rAAV (ssAAV).

50-53. (canceled)

54. The method of claim 11, further comprising administering a therapeutically effective amount of a glucocorticoid and/or anti-histamine to the subject.

55. (canceled)

56. The method of claim 54, wherein the glucocorticoid is prednisolone, methylprednisolone, or a combination thereof; and the anti-histamine is diphenhydramine, hydroxyzine, chlorpheniramine, or any combination thereof.

57-59. (canceled)

60. The method of claim 11, wherein after administering the rAAV vector, N-acetylaspartate (NAA) levels in urine, cerebrospinal fluid (CSF), and/or brain tissue are decreased.

61-70. (canceled)
